(19) United States
(12) Patent Application Publication
(10) Pub. No.: US 2019/0381171 A1
(43) Pub. Date: Dec. 19, 2019

(54) CO-USE OF ANTI-BCMA ANTIBODY AND ANTIBODY-COUPLED T CELL RECEPTOR (ACTR) IN CANCER THERAPY AND B CELL DISORDERS

(71) Applicant: UNUM THERAPEUTICS INC., Cambridge, MA (US)

(72) Inventors: Heather HUET, Cambridge, MA (US); Seth ETTENBERG, Cambridge, MA (US); Django SUSSMAN, Bothell, WA (US); Tooba CHEEMA, Cambridge, MA (US); Taylor HICKMAN, Cambridge, MA (US); Katie O'CALLAGHAN, Glasheen, Cork (IE); Maureen RYAN, Bothell, WA (US)

(73) Assignee: UNUM THERAPEUTICS INC., Cambridge, MA (US)

(21) Appl. No.: 16/486,741

(22) PCT Filed: Feb. 16, 2018
(86) PCT No.: PCT/US18/00028

§ 371 Date: Aug. 16, 2019

Related U.S. Application Data
(60) Provisional application No. 62/460,669, filed on Feb. 17, 2017.

(51) Int. Cl. A61K 39/395; C07K 16/28; A61K 35/17; A61K 31/454; C07K 16/30; A61P 35/00

(52) U.S. Cl. CPC A61K 39/39558; C07K 16/2878; A61K 39/3955; A61K 35/17; A61K 31/454; A61K 2039/505; A61P 35/00; C07K 2317/732; C07K 2317/41; C07K 2317/24; C07K 16/30

(57) ABSTRACT

Disclosed herein are methods of enhancing antibody-dependent cell cytotoxicity (ADCC) in a subject comprising administering to a subject in need thereof a therapeutically effective amount of an anti-BCMA antibody and an effective amount of T lymphocytes and/or NK cells expressing an antibody-coupled T-cell receptor (ACTR) construct, which may comprises an extracellular domain with affinity and specific for the Fc portion of an immunoglobulin molecule (Ig); a transmembrane domain; optionally one or more of co-stimulatory domains, and a cytoplasmic signaling domain comprising an immunoreceptor tyrosine-based activation motif (ITAM).

### CROSS REFERENCE TO RELATED APPLICATIONS

This application claims the benefit of the filing date of U.S. Provisional Application No. 62/460,669, filed Feb. 17, 2017, the entire contents of which is incorporated by reference herein.

### BACKGROUND OF DISCLOSURE

Cancer immunotherapy, including cell-based therapy, antibody therapy and cytokine therapy, is used to provoke immune responses attacking abnormal cells (such as tumor cells) while sparing normal tissues. It is a promising option for treating various types of cancer because of its potential to evade genetic and cellular mechanisms of drug resistance, and to target tumor cells while sparing normal tissues. T-lymphocytes can exert major anti-tumor effects as demonstrated by results of allogeneic hematopoietic stem cell transplantation (HSCT) for hematologic malignancies, where T-cell-mediated graft-versus-host disease (GvHD) is inversely associated with disease recurrence, and immunosuppression withdrawal or infusion of donor lymphocytes can contain relapse. Weiden et al., N Engl J Med. 1979; 300(19):1068-1073; Porter et al., N Engl J Med. 1994; 330(2):100-106; Kolb et al., Blood. 1995; 86(5):2041-2050; Slavin et al., Blood. 1996; 87(6):2195-2204; and Appelbaum, Nature. 2001; 411(6835):385-389. It is also a promising treatment for B cell and plasma cell mediated diseases such as autoimmune disorders and inflammatory disorders.

Cell-based therapy may involve cytotoxic T cells having reactivity skewed toward cancer cells, B cells, and plasma cells. Eshhar et al., Proc. Natl. Acad. Sci. U.S.A.; 1993; 90(2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric receptor having an antigen-binding domain fused to one or more T cell activation signaling domains. Binding of a cancer, B cell, or plasma cell antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14(11):1264-1270; Porter et al., N Engl J Med; 2011; 25; 365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013; 5(177):177ra138.

Another approach is to express an Antibody-coupled T cell Receptor (ACTR) protein in an immune cell, such as an NK cell or a T cell. The ACTR protein contains an extracellular Fc-binding domain. When the ACTR-expressing T cells (also called “ACTR T cells”) are administered to a subject together with an antibody targeting specific types of cells, for example, cancer cells, they may enhance toxicity against the targeted cells, which are targeted by the antibody via their binding to the Fc domain of the antibody. Kudo et al., Cancer Research (2014) 74:93-103.

Antibody-based immunotherapies, such as monoclonal antibodies, antibody-fusion proteins, and antibody drug conjugates (ADCs) are used to treat a wide variety of diseases, including many types of cancer, B cell mediated disorders, and plasma cell mediated disorders. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a target cell can lead to target cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

### SUMMARY OF DISCLOSURE

The present disclosure is based, at least in part, on the development of methods for enhancing antibody-dependent cell cytotoxicity (ADCC) in a subject, involving the co-use of immune cells expressing an antibody-coupled T-cell receptor (ACTR) and an antibody specific to B-cell maturation antigen (BCMA). T cells expressing the improved ACTR constructs described herein in combination with an anti-BCMA antibody demonstrated superior in vivo and in vitro bioactivities including cytotoxicity, cell proliferation and activation (e.g., IL-2 production, percentage of CD25+ and/or CD69+ cells), and/or in vivo anti-cancer activity (e.g., anti-BCMA positive target cell such as multiple myeloma cell activity).

Accordingly, one aspect of the present disclosure provides a method of enhancing antibody-dependent cell cytotoxicity (ADCC) in a subject, the method comprising administering to a subject in need thereof a therapeutically effective amount of an anti-BCMA antibody and an effective amount of immune cells, which can be T lymphocytes or NK cells expressing an antibody-coupled T-cell receptor (ACTR) construct.

The ACTR construct may comprise: (a) an Fc binding domain; (b) a transmembrane domain; (c) a cytoplasmic signaling domain comprising an immunoreceptor tyrosine-based activation motif (ITAM) and optionally (d) at least one co-stimulatory signaling domain. Either the cytoplasmic signaling domain (c) or the at least one co-stimulatory signaling domain (d) can be located at the C-terminus of the ACTR construct. In some examples, the cytoplasmic signaling domain can be located at the C-terminus of the ACTR construct.

In some embodiments, the ACTR construct may further comprise (e) a hinge domain, which can be located at the C-terminus of (a) and the N-terminus of (b). Alternatively or in addition, the ACTR construct may further comprise a signal peptide at its N-terminus, which may be from CD8α.

In some embodiments, the Fc binding domain (a) in the ACTR construct can be (i) an extracellular ligand-binding domain of an Fc-receptor, which optionally is an Fc-gamma receptor, an Fc-alpha receptor, or an Fc-epsilon receptor, (ii) an antibody fragment that binds the Fc portion of an immunoglobulin, (iii) a naturally-occurring protein that binds the Fc portion of an immunoglobulin, or an Fc-binding fragment thereof, or (iv) a synthetic polypeptide that binds the Fc portion of an immunoglobulin. In some examples, the Fc binding domain is an extracellular ligand-binding domain of a CD16A, CD32A, or CD64A receptor. In certain embodiments, the extracellular ligand-binding domain of CD16A is CD16A F158 (a.k.a., F158 FCGR3A) or CD16A V158 (a.k.a., V158 FCGR3A). In other examples, the Fc binding domain is (ii), which is a single chain variable fragment (ScFv), a domain antibody, or a nanobody. In yet other examples, the Fc binding domain is (iii), which is Protein A or Protein G. Alternatively, the Fc binding domain is (iv), which is a Kunitz peptide, a SMIP, an avimer, an affibody, a DARPin, or an anticalin.

In some embodiments, the transmembrane domain (b) in the ACTR construct can be of a single-pass membrane protein. For example, the transmembrane domain is of CD8α, CD8β, 4-1BB, CD28, CD34, CD4, FcεRIγ, CD16A, OX40, CD3ζ, CD3ε, CD3γ, CD3δ, TCRα, CD32, CD64, VEGFR2, FAS, PD1, or FGFR2B. Alternatively, the transmembrane domain of (b) can be a non-naturally occurring hydrophobic protein segment.

In some embodiments, the at least one co-stimulatory signaling domain (d) in the ACTR can be of 4-1BB, CD28, CD28_{LL→GG} variant, OX40, ICOS, CD27, GITR, HVEM, TIM1, LFA1, or CD2. In some examples, the ACTR construct described herein may comprise a single co-stimulatory signaling domain (as the only co-stimulatory signaling domain in the ACTR construct) or two co-stimulatory signaling domains. Exemplary co-stimulatory signaling domain combinations include, but are not limited to, (i) CD28 and 4-1BB; (ii) CD28_{LL→GG} variant and 4-1BB; (iii) CD28 and CD27; or (iv) CD28 and OX40.

In some embodiments, the cytoplasmic signaling domain (c) in the ACTR construct can be a cytoplasmic domain of CD3ζ or FcεR1γ.

In some embodiments, the hinge domain (e) can be of CD8α, CD28, or IgG. Alternatively, the hinge domain can be a non-naturally occurring peptide, e.g., an extended recombinant polypeptide (XTEN) or a (Gly₄Ser)ₙ polypeptide, in which n is an integer of 3-12, inclusive. In some embodiments, the hinge domain is 1 to 60 amino acid residues in length, for example, 1 to 30 amino acid residues in length or 31 to 60 amino acid residues in length.

In some embodiments, the ACTR construct comprises (i) a single co-stimulatory domain of 4-1BB or CD28, and (ii) a hinge domain of CD8 or CD28, a transmembrane domain of CD8 or CD28, or a combination thereof. In some embodiments, the ACTR construct comprises (i) a 4-1BB co-stimulatory signaling domain, and (ii) a CD8 hinge domain, a CD8 transmembrane domain, or a combination thereof. In certain embodiments, the ACTR construct comprises (i) a CD28 co-stimulatory signaling domain, and (ii) a CD28 hinge domain, a CD28 transmembrane domain, or a combination thereof. In specific embodiments, the ACTR construct is free of a hinge domain from any non-CD16A receptor. In some embodiments, the ACTR construct is free of any hinge domain. In certain embodiments, the ACTR construct comprises a CD28 co-stimulatory signaling domain and optionally a CD8 transmembrane domain

In some specific examples, the ACTR construct described herein comprises: (a) an extracellular ligand-binding domain of F158 FCGR3A or V158 FCGR3A, (b) a hinge and transmembrane domain of CD8α, (c) a cytoplasmic signaling domain of CD3ζ, and optionally (d) a co-stimulatory signaling domain of 4-1BB, wherein either (c) or (d) is located at the C-terminus of the ACTR construct. For example, the ACTR construct may comprise the amino acid sequence of residues 22 to 436 of SEQ ID NO: 1, or residues 22 to 436 of SEQ ID NO: 31. In one particular example, the ACTR construct comprises the amino acid sequence of SEQ ID NO: 1 or SEQ ID NO:31.

In some specific examples, the ACTR construct described herein comprises: (a) an extracellular ligand-binding domain of F158 FCGR3A or V158 FCGR3A, (b) a hinge and transmembrane domain of CD28, (c) a cytoplasmic signaling domain of CD3, and optionally (d) a co-stimulatory signaling domain of CD28, wherein either (c) or (d) is located at the C-terminus of the ACTR construct. For example, the ACTR construct may comprise the amino acid sequence of residues 22-432 of SEQ ID NO: 57. In one particular example, the ACTR construct comprises the amino acid sequence of SEQ ID NO: 57.

In additional specific examples, the ACTR construct described herein comprises: (a) an extracellular ligand-binding domain of F158 FCGR3A or V158 FCGR3A, (b) a transmembrane domain of CD8α, (c) a cytoplasmic signaling domain of CD3ζ, and optionally (d) a co-stimulatory signaling domain of CD28, wherein either (c) or (d) is located at the C-terminus of the ACTR construct. For example, the ACTR construct may comprise the amino acid sequence of residues 22-390 of SEQ ID NO: 58. In one particular example, the ACTR construct comprises the amino acid sequence of SEQ ID NO: 58.

In any of the methods described herein, the subject to be treated is a human patient having a BCMA-positive cancer. As used herein, the term “BCMA-positive cancer” refers to a cancer having cancer cells that express B-cell maturation antigen (BCMA) also known as TNFRSF17. In some embodiments, the BCMA-positive cancer is a hematological cancer, including, but not limited to, myeloma, leukemia, or lymphoma. In some embodiments, the subject is a human patient having a B cell mediated disorder or a plasma cell mediated disorder, including, but not limited to, rheumatoid arthritis, systemic lupus E (SLE), Type I diabetes, asthma, atopic dermatitis, allergic rhinitis, thrombocytopenic purpura, multiple sclerosis, psoriasis, Sjögren's syndrome, Hashimoto's thyroiditis, Graves' disease, primary biliary cirrhosis, Granulomatosis with polyangiitis (GPA), tuberculosis, or graft-vs-host disease (GVHD).

The T lymphocytes or NK cells for use in any of the methods described herein can be autologous T lymphocytes or autologous NK cells isolated from the subject. Alternatively, the T lymphocytes or NK cells for use in any of the methods described herein can be allogeneic T lymphocytes or allogeneic NK cells. In some embodiments, the autologous T lymphocytes, autologous NK cells, allogeneic T lymphocytes, or allogeneic NK cells can be activated and/or expanded ex vivo prior to administering into the subject. In certain embodiments, the ex vivo activation and/or expansion may be performed in the presence of an immunomodulatory agent (e.g., lenalidomide).

In some examples, the allogeneic T lymphocytes are T lymphocytes in which the expression of the endogenous T cell receptor has been inhibited or eliminated.

In any of the methods described herein, the ACTR construct can be introduced into the T lymphocytes or the NK cells by a method selected from the group consisting of retroviral transduction, lentiviral transduction, DNA electroporation, and RNA electroporation.

In any of the methods described herein, the T lymphocytes or NK cells expressing the ACTR construct can be co-administered with an immunomodulatory agent (e.g., lenalidomide).

In any of the methods described herein, the anti-BCMA antibody can be a human or humanized antibody, which may comprise an IgG1 heavy chain constant region. In any of the methods described herein, the anti-BCMA antibody can be a chimeric antibody. In certain embodiments, the anti-BCMA antibody may contain an Fc variant fragment which has elevated binding affinity to a wild-type Fc receptor (e.g., when compared to a wild-type IgG such as that found in rituximab or trastuzumab). In certain embodiments, the anti-BCMA antibody may have one or more mutations in the hinge and/or CH2 domain of the antibody. Alternatively or in addition, the anti-BCMA antibody may be glycoengineered (e.g., may be afucosylated).

In some embodiments, the anti-BCMA antibody used in any of the methods described herein binds to the same epitope in BCMA as antibody hSG16.17 or hSG16.45, or competes against antibody hSG16.17 or hSG16.45 from binding to BCMA. In some examples, the anti-BCMA antibody comprises the same heavy chain complementary determining regions (CDRs) and light chain CDRs as antibody hSG16.17 or hSG16.45. For example, the anti-BCMA antibody may comprise the same heavy chain variable domain (V_{H}) and the same light chain variable domain (V_{L}) as antibody hSG16.17 or hSG16.45.

In another aspect, provided herein is a kit for immunotherapy, comprising: (i) a pharmaceutical composition that comprises any of the anti-BCMA antibodies as defined herein, and a pharmaceutically acceptable carrier; and (ii) a population of T lymphocytes or NK cells that express an antibody-coupled T-cell receptor (ACTR) construct as defined herein. In some embodiments, the kit may further comprise an immunomodulatory agent (e.g., lenalidomide).

Also within the scope of the present disclosure are pharmaceutical compositions comprising any of the anti-BCMA antibodies for use in treating any of the target diseases described herein concurrently with any of the immune cells expressing an ACTR construct; and uses of the anti-BCMA antibodies and immune cells in manufacturing a medicament for use in treating the target disease.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a set of graphs showing the amount of BCMA on different tumor cell samples as determined by flow cytometry using the SG16.17 anti-BCMA antibody. Measurements are shown as geometric Mean Fluorescence Intensity (gMFI). A: multiple myeloma cell lines (NCI-H929, U266B1, RPMI8226, MM1S, OPM2) and K562 control cells. B: cell lines KMS-20, NCI-H929 and U266B1 cells and two samples of Multiple Myeloma (MM) patient-derived bone marrow mononuclear cells (BMMC).

FIG. 2 is a set of graphs showing the binding curves of chimeric (A) and humanized (B) versions of the anti-BCMA SG16.17 and SG16.17SEA (afucosylated) antibodies to ACTR variant SEQ ID NO: 1 T cells by flow cytometry. The geometric mean fluorescence intensities (gMFI) of the PE signal from the anti-Fc detection antibody are shown for increasing concentrations of each anti-BCMA antibody.

FIG. 3 is a set of graphs showing the antibody specific cytotoxicity of BCMA-expressing NCI-H929 (A) and U266B1 (B) target cells induced by T-cells expressing ACTR variant SEQ ID NO: 1 in combination with anti-BCMA chimeric SG16.17, chimeric SG16.45, chimeric SG16.17SEA (afucosylated), and chimeric SG16.45SEA (afucosylated) antibodies.

FIG. 4 is a set of graphs demonstrating IFNγ (A) and IL-2 (B) production by T-cells expressing ACTR variant SEQ ID NO: 1 incubated with BCMA-expressing NCI-H929 target cells and anti-BCMA chimeric SG16.17, chimeric SG16.45, chimeric SG16.17SEA (afucosylated), and chimeric SG16.45SEA (afucosylated) antibodies.

FIG. 5 is a set of graphs showing antibody specific cytotoxicity of BCMA-expressing target cells (NCI-H929 cells) incubated with T-cells expressing ACTR variant SEQ ID NO: 1 in combination with anti-BCMA chimeric SG16.17, humanized SG16.17 #1, humanized SG16.17 #9, humanized SG16.17 #10, chimeric SG16.45, humanized SG16.45 #1, humanized SG16.45 #3, and humanized SG16.45 #18 antibodies. A: results using anti-BCMA chimeric SG16.17, humanized SG16.17 #1, humanized SG16.17 #9, and humanized SG16.17 #10 antibodies. B: results using anti-BCMA chimeric SG16.45, humanized SG16.45 #1, humanized SG16.45 #3, and humanized SG16.45 #18 antibodies.

FIG. 6 is a set of graphs showing antibody specific cytotoxicity of BCMA-expressing NCI-H929 (A) and U266B1 (B) target cells incubated with T-cells expressing ACTR variant SEQ ID NO: 1 in combination with anti-BCMA humanized SG16.17 antibody, humanized SG16.17SEA (afucosylated) antibody, and an antibody derived from the NCI-murine BCMA CAR sequence shown in WO2010104949A2.

FIG. 7 is a set of graphs demonstrating the results of experiments to assess Jurkat cell activation by an afucosylated humanized anti-BCMA antibody (hSG16.17SEA) at various concentrations for cell lines that express BCMA (NCI-H929, U266B1, RPMI8226, OPM2, MM1s) and cell lines that do not express BCMA (K562). A: Results from Jurkat cells with the NFAT reporter system (Jurkat-N) transduced with retrovirus expressing ACTR (Jurkat-N-ACTR). B: Results from Jurkat cells with the NFAT reporter system, but lacking ACTR expression (Jurkat-N), which were used as a negative control.

FIG. 8 is a set of graphs showing antibody specific cytotoxicity of BCMA-expressing NCI-H929 (A) and U266B1 (B) target cells incubated with T-cells expressing ACTR variant SEQ ID NO: 1 or Mock T cells in combination with afucosylated humanized anti-BCMA antibody (hSG16.17SEA).

FIG. 9 is a graph demonstrating IFNγ and IL2 production by T-cells expressing ACTR variant SEQ ID NO: 1 incubated with BCMA-expressing target cells (NCI-H929 target cells) and afucosylated humanized anti-BCMA antibody (hSG16.17SEA).

FIG. 10 includes a series of graphs demonstrating the proliferation level of CD3 positive T cells at days 1 and 7 when ACTR T variant SEQ ID NO: 1 cells were co-incubated with BCMA expressing MM target cell lines NCI-H929 (A); U266B1 (C), and RPMI-8226 (D) in the presence of afucosylated humanized anti-BCMA antibody (hSG16.17SEA). No expansion of the level of CD3+ T cells was seen when NCI-H929 target cells were co-incubated with control Mock T cells (B).

FIG. 11 is a graph demonstrating the level of expansion of CD3-positive cells using ACTR variant SEQ ID NO: 1 after repeated stimulation (every 3-4 days) with BCMA-positive NCI-H929 target cells in the presence of afucosylated humanized anti-BCMA antibody (hSG16.17SEA); no T cell expansion was observed in the presence of BCMA-negative K562 cells under the same conditions. No expansion of CD3+ T cells was seen when NCI-H929 or K562 target cells were co-incubated with control Mock T cells in the presence of afucosylated humanized anti-BCMA antibody (hSG16.17SEA).

FIG. 12 is a set of graphs demonstrating tumor growth and time to tumor volume end-point in a subcutaneous NCI-H929 model of multiple myeloma in NSG mice. Treatment groups were as follows: vehicle (PBS alone), hSG16.17SEA antibody alone, ACTR variant SEQ ID NO: 1 T-cells alone, and ACTR variant SEQ ID NO: 1 T-cells in combination with hSG16.17SEA antibody. Groups treated with hSG16.17SEA antibody were dosed with 100 μg of antibody per mouse once a week for 4 weeks (QWx4, represented by dotted vertical lines); groups treated with ACTR T-cells were dosed with 1×10⁷ cells per mouse dosed once a week for 2 weeks (QWx2, represented by grey arrows). A: The mean tumor volume was plotted as a function of time for mice in the different treatment arms. B: The percentage of mice remaining on study was plotted as a function of time to predefined tumor volume end-point of mice in the different treatment arms.

FIG. 13 is a set of graphs showing cytotoxicity of BCMA-expressing NCI-H929 (A) and U266B1 (B) target cells incubated with T-cells expressing ACTR variant SEQ ID NO: 1 or Mock T cells at varying effector T cell to Target cell (E:T) ratios. These are performed in the presence of 1 μg/mL of various chimeric anti-BCMA antibodies or an isotype control antibody.

FIG. 14 is a set of graphs demonstrating levels of CD25 (A) and CD69 (B) activation markers on CD3 positive cells of ACTR variant SEQ ID NO: 1 after co-culture with BCMA-positive target cells (NCI-H929 and U266B1) and in the presence of 1 μg/mL of various chimeric anti-BCMA antibodies, or an isotype control antibody.

FIG. 15 is a set of graphs showing IFNγ production by mock T cells and T cells expressing ACTR variant SEQ ID NO: 1 in the presence of (A) BCMA+ HER2− NCI-H929 or (B) BCMA− HER2+ SKBR3 target cells and increasing concentrations of anti-BCMA (hSG16.17 SEA) or anti-HER2 (trastuzumab) antibodies. Robust cytokine production was observed only with ACTR T-cells in combination with a matched antibody/target pair; low or no cytokine production was observed with mock T cells or with ACTR T cells in the presence of mismatched antibody/target pairs.

FIG. 16 is a set of graphs showing the cytotoxicity of BCMA-expressing NCI-H929 target cells induced by T-cells expressing (A) ACTR variant SEQ ID NO: 57 or (B) ACTR variant SEQ ID NO: 58. Mock T cells were included as a control in this experiment. ACTR-T-cell-mediated cytotoxicity was only observed in the presence of hSG16.17 SEA antibody and increased with increasing effector-to-target (E:T) ratio.

FIG. 17 is a set of graphs showing the activation, as measured by CD25 expression, of T-cells expressing (A) ACTR variant SEQ ID NO: 57 and (B) ACTR variant SEQ ID NO: 58 in the absence and presence of hSG16.17 SEA antibody and NCI-H929 target cells. Mock T cells were included as a control in this experiment. The expression of the activation marker, CD25, increased on the surface of T-cells expressing ACTR variants SEQ ID NO: 57 and SEQ ID NO: 58 in the presence of NCI-H929 target cells and hSG16.17 SEA antibody but not in the absence of antibody. No increase in CD25 on mock T cells is observed with or without antibody.

FIG. 18 is a set of graphs showing antibody specific cytotoxicity of BCMA-expressing NCI-H929 (A) target cells and BCMA-negative K562 (B) target cells induced by T-cells expressing ACTR variant SEQ ID NO: 57 and SEQ ID NO: 58. Mock T cells were included as a negative control in this experiment. Cytotoxicity mediated by ACTR T-cells was observed in the presence of hSG16.17 SEA antibody in a concentration dependent manner only with BCMA-expressing target cells.

FIG. 19 is a set of graphs demonstrating IFNγ (A) and IL2 (B) cytokine production by T-cells expressing ACTR variants SEQ ID NO: 57 and SEQ ID NO: 58 incubated with BCMA-expressing target cells (NCI-H929 target cells) and anti-BCMA antibody hSG16.17SEA antibody at varying concentrations. Mock T cells were included as a negative control in this experiment. T cells expressing ACTR variants SEQ ID NO: 57 and SEQ ID NO: 58 showed a concentration-dependent increase in IFNγ and IL2 production; mock T cells showed no detectable cytokine production.

FIG. 20 is a graph demonstrating the proliferation level of CD3 positive T cells at day 7 when either Mock T cells or ACTR variant SEQ ID NO: 57 cells were co-incubated with BCMA-expressing NCI-H929 cells in the presence of varying concentrations of anti-BCMA antibody hSG16.17SEA. Proliferation of ACTR T cells was observed only in the presence of antibody; no expansion of CD3+ T cells was seen when NCI-H929 target cells were co-incubated with control Mock T cells.

FIG. 21 is a set of graphs showing the proliferation of T-cells expressing ACTR variant SEQ ID NO:1 in the presence and absence of lenalidomide with (A) NCI-H929 and (B) RPMI-8226 target cells. Mock T-cells were used as a control in this experiment. Proliferation was only observed when ACTR T-cells were cultured in the presence of hSG16.17 SEA antibody and was enhanced in the presence of 1 μM lenalidomide.

### DETAILED DESCRIPTION OF DISCLOSURE

Antibody-based immunotherapies are used to treat a wide variety of diseases, including many types of cancer. Such a therapy often depends on recognition of cell surface molecules that are differentially expressed on cells for which elimination is desired (e.g., target cells such as cancer cells) relative to normal cells (e.g., non-cancer cells) (Weiner et al. Cell (2012) 148(6): 1081-1084). Several antibody-based immunotherapies have been shown in vitro to facilitate antibody-dependent cell-mediated cytotoxicity of target cells (e.g. cancer cells), and for some it is generally considered that this is the mechanism of action in vivo, as well. ADCC is a cell-mediated innate immune mechanism whereby an effector cell of the immune system, such as natural killer (NK) cells, T cells, monocyte cells, macrophages, or eosinophils, actively lyses target cells (e.g., cancer cells) recognized by specific antibodies.

The present disclosure is based, at least in part, on the unexpected findings that the combined use of anti-BCMA antibodies and ACTR T cells successfully enhanced cell death in BCMA-positive cancer cells. These findings indicate that BCMA is a suitable target for antibody/ACTR-T cell combined therapy.

Accordingly, the present disclosure provides a method of enhancing antibody-dependent cell cytotoxicity (ADCC) in a subject using a combination therapy comprising a therapeutically effective amount of an anti-BCMA antibody and a therapeutically effective amount of immune cells (e.g., T lymphocytes or NK lymphocytes), that express an ACTR construct as described herein, and a kit for immunotherapy, comprising the anti-BCMA antibody and the ACTR-expressing immune cells as described herein. The T lymphocytes and/or NK cells of the method or kit may further express an exogenous polypeptide comprising a co-stimulatory domain or a ligand of a co-stimulatory factor, which provides a co-stimulatory signal in trans. Alternatively, the exogenous polypeptide may comprise a check-point receptor or a functional domain thereof.

As used herein, an ACTR construct refers to a non-naturally occurring molecule that can be expressed on the surface of a host cell and comprises an extracellular domain capable of binding to a target molecule containing an Fc portion and one or more cytoplasmic signaling domains for triggering effector functions of the immune cell expressing the ACTR construct, wherein at least two domains of the ACTR construct may be derived from different molecules.

B-cell maturation antigen (BCMA) is a member of the tissue necrosis factor receptor (TNFR) superfamily expressed on B cells, particularly mature B cells. BCMA binds to a proliferation-inducing ligand of the TNFR superfamily, leading to activation of the NFκB and MAPK8/JNK signaling pathways. It is suggested that BCMA plays an important role in B cell development and autoimmune responses. As an example, a human BCMA protein was provided in GenBank under accession number BAB60895 (shown below as SEQ ID NO: 71).

Anti-BCMA antibodies can bind to a BCMA protein expressed on the surface of a target cell (e.g., a BCMA-positive cancer cell, a B cell, and a plasma cell). Immune cells that express receptors capable of binding such Fc-containing molecules, for example the ACTR construct molecules described herein, recognize the target cell-bound anti-BCMA antibodies and this receptor/antibody engagement stimulates the immune cell to perform effector functions such as release of cytotoxic granules or expression of cell-death-inducing molecules, leading to enhanced cell toxicity of the BCMA-expressing target cells.

The method described herein would confer a number of advantages. For example, via the extracellular domain that binds Fc, the ACTR constructs described herein can bind to the Fc portion of the anti-BCMA antibodies rather than directly binding a specific target antigen (e.g., a cancer antigen). Thus, immune cells expressing the ACTR constructs described herein would be able to induce/enhance cell death of any type of BCMA-positive cells that are bound by the anti-BCMA antibody.

### I. ACTR Constructs

The ACTR constructs described herein comprise an extracellular domain with binding affinity and specificity for the Fc portion of an immunoglobulin (“Fc binder” or “Fc binding domain”), a transmembrane domain, and a cytoplasmic signaling domain comprising an immunoreceptor tyrosine-based activation motif (ITAM). In some embodiments, the ACTR constructs described herein may further include at least one co-stimulatory signaling domain. The ACTR constructs are configured such that, when expressed on a host cell, the extracellular ligand-binding domain is located extracellularly for binding to a target molecule (e.g., an anti-BCMA antibody) and the ITAM-containing cytoplasmic signaling domain. The optional co-stimulatory signaling domain may be located in the cytoplasm for triggering activation and/or effector signaling. In some embodiments, an ACTR construct as described herein may comprise, from N-terminus to C-terminus, the Fc binding domain, the transmembrane domain, and the ITAM-containing cytoplasmic signaling domain. In some embodiments, an ACTR construct as described herein comprises, from N-terminus to C-terminus, the Fc binding domain, the transmembrane domain, at least one co-stimulatory signaling domain, and the ITAM-containing cytoplasmic signaling domain. In other embodiments, an ACTR construct as described herein comprises, from N-terminus to C-terminus, the Fc binding domain, the transmembrane domain, the ITAM-containing cytoplasmic signaling domains, and at least one co-stimulatory signaling domain.

Any of the ACTR constructs described herein may further comprise a hinge domain, which may be located at the C-terminus of the Fc binding domain and the N-terminus of the transmembrane domain. Alternatively or in addition, the ACTR constructs described herein may contain two or more co-stimulatory signaling domains, which may link to each other or be separated by the ITAM-containing cytoplasmic signaling domain. The extracellular Fc binder, transmembrane domain, optional co-stimulatory signaling domain(s), and ITAM-containing cytoplasmic signaling domain in an ACTR construct may be linked to each other directly, or via a peptide linker. In some embodiments, any of the ACTR constructs described herein may comprise a signal sequence at the N-terminus.

Some exemplary ACTR constructs are provided in WO2016040441, the relevant disclosures therein are incorporated by reference for the purpose and subject matter referenced herein.

As used in this specification and the appended claims, the singular forms “a,” “an,” and “the” include plural references unless the context clearly dictates otherwise.

### A. Fc Binding Domains

The ACTR constructs described herein comprise an extracellular domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the anti-BCMA antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or a nanobody. Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binding domain is an extracellular ligand-binding domain of a mammalian Fc receptor. As used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR construct as described herein may be a naturally-occurring polymorphism variant (e.g., the CD16 V158 variant), which may have increased or decreased affinity to Fc as compared to a wild-type counterpart (e.g., CD16 F158). Alternatively, the Fc receptor may be a functional variant of a wild-type counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc.

The table below lists a number of exemplary polymorphisms in Fc receptor extracellular domains (see, e.g., Kim et al., J. Mol. Evol. 53:1-9, 2001) which may be used in any of the methods or constructs described herein:

TABLE 1
Exemplary Polymorphisms in Fc Receptors

| | Amino Acid Number | | | | | | | | | |
|---|---|---|---|---|---|---|---|---|---|---|
| | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 | N | N | N | S | E | S | S | S | I | I |
| (FcγRII) | | | | | | | | | | |
| M31932 | S | T | N | R | E | A | F | T | I | G |
| (FcγRI) | | | | | | | | | | |
| X06948 | R | S | E | S | Q | S | E | S | I | V |
| (FcαεI) | | | | | | | | | | |

Fe receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fe-alpha receptor is FcαR1/CD89. Examples of Fe-epsilon receptors include, without limitation, FcεRI and FcεRII/CD23. The table below lists exemplary Fc receptors for use in constructing the ACTR constructs described herein and their binding activity to corresponding Fc domains:

TABLE 2
Exemplary Fc Receptors

| Receptor name | Principal antibody ligand | Affinity for ligand |
|---|---|---|
| FcγRI (CD64) | IgG1 and IgG3 | High (Kd ~ 10⁻⁹M) |
| FcγRIIA (CD32) | IgG | Low (Kd > 10⁻⁷M) |
| FcγRIIB1 (CD32) | IgG | Low (Kd > 10⁻⁷M) |
| FcγRIIB2 (CD32) | IgG | Low (Kd > 10⁻⁷M) |
| FcγRIIIA (CD16a) | IgG | Low (Kd > 10⁻⁶M) |
| FcγRIIIB (CD16b) | IgG | Low (Kd > 10⁻⁶M) |
| FcεRI | IgE | High (Kd ~ 10⁻¹⁰M) |
| FcεRII (CD23) | IgE | Low (Kd > 10⁻⁷M) |
| FcαRI (CD89) | IgA | Low (Kd > 10⁻⁶M) |
| Fcα/μR | IgA and IgM | High for IgM, Mid for |
| FcRn | IgG | IgA |

Selection of the ligand binding domain of an Fc receptor for use in the ACTR constructs described herein will be apparent to one of skill in the art. For example, it may depend on factors such as the isotype of the anti-BCMA antibody to which binding of the Fc receptor is desired and the desired affinity of the binding interaction.

In some examples, (a) is the extracellular ligand-binding domain of CD16, which may incorporate a naturally occurring polymorphism that may modulate affinity for Fc. In some examples, (a) is the extracellular ligand-binding domain of CD16 incorporating a polymorphism at position 158 (e.g., valine or phenylalanine). In some embodiments, (a) is produced under conditions that alter its glycosylation state and its affinity for Fc.

In some embodiments, (a) is the extracellular ligand-binding domain of CD16 incorporating modifications that render the ACTR construct specific for a subset of IgG antibodies. For example, mutations that increase or decrease the affinity for an IgG subtype (e.g., IgG1) may be incorporated.

In some examples, (a) is the extracellular ligand-binding domain of CD32, which may incorporate a naturally occurring polymorphism that may modulate affinity for Fc. In some embodiments, (a) is produced under conditions that alter its glycosylation state and its affinity for Fc.

In some embodiments, (a) is the extracellular ligand-binding domain of CD32 incorporating modifications that render the ACTR construct specific for a subset of IgG antibodies. For example, mutations that increase or decrease the affinity for an IgG subtype (e.g., IgG1) may be incorporated.

In some examples, (a) is the extracellular ligand-binding domain of CD64, which may incorporate a naturally occurring polymorphism that may modulate affinity for Fc. In some embodiments, (a) is produced under conditions that alter its glycosylation state and its affinity for Fc.

In some embodiments, (a) is the extracellular ligand-binding domain of CD64 incorporating modifications that render the ACTR construct specific for a subset of IgG antibodies. For example, mutations that increase or decrease the affinity for an IgG subtype (e.g., IgG1) may be incorporated.

In other embodiments, the Fc binding domain is derived from a naturally occurring bacterial protein that is capable of binding to the Fc portion of an IgG molecule. A Fc binding domain for use in constructing an ACTR construct as described herein can be a full-length protein or a functional fragment thereof. Protein A is a 42 kDa surface protein originally found in the cell wall of the bacterium Staphylococcus aureus. It is composed of five domains that each fold into a three-helix bundle and are able to bind IgG through interactions with the Fc region of most antibodies as well as the Fab region of human VH3 family antibodies. Protein G is an approximately 60-kDa protein expressed in group C and G Streptococcal bacteria that binds to both the Fab and Fc region of mammalian IgGs. While native protein G also binds albumin, recombinant variants have been engineered that eliminate albumin binding.

Fc binding domains for use in ACTR constructs may also be created de novo using combinatorial biology or directed evolution methods. Starting with a protein scaffold (e.g., an scFv derived from IgG, a Kunitz domain derived from a Kunitz-type protease inhibitor, an ankyrin repeat, the Z domain from protein A, a lipocalin, a fibronectin type III domain, an SH3 domain from Fyn, or others), amino acid side chains for a set of residues on the surface may be randomly substituted in order to create a large library of variant scaffolds. From large libraries it is possible to isolate rare variants with affinity for a target like the Fc domain by first selecting for binding, followed by amplification by phage, ribosome or cell display. Repeated rounds of selection and amplification can be used to isolate those proteins with the highest affinity for the target. Fc-binding peptides are known in the art, e.g., DeLano et al., Science, 287:5456 (2000); Jeong et al., Peptides, 31(2):202-206 (2009); and Krook et al., J. Immunological Methods, 221(1-2):151-157 (1998). Exemplary Fc-binding peptides may comprise the amino acid sequence of ETQRCTWHMGELVWCEREHN (SEQ ID NO:85), KEASCSYWLGELVWCVAGVE (SEQ ID NO:86), or DCAWHLGELVWCT (SEQ ID NO:87).

Any of the Fc binding domains described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the ACTR constructs described herein may have a binding affinity K_{D} of at least 10⁻⁵, 10⁻⁶, 10⁻⁷, 10⁻⁸, 10⁻⁹, 10⁻¹⁰M or lower for the Fc portion of antibody. In some embodiments, the Fc binding domain has a high binding affinity for antibody, isotype of antibodies, or subtype(s) thereof, as compared to the binding affinity of the Fc binding domain to another antibody, isotype of antibodies or subtypes thereof. In some embodiments, the extracellular ligand-binding domain of an Fc receptor has specificity for an antibody, isotype of antibodies, or subtype(s) thereof, as compared to binding of the extracellular ligand-binding domain of an Fc receptor to another antibody, isotype of antibodies, or subtypes thereof. Fe-gamma receptors with high affinity binding include CD64A, CD64B, and CD64C. Fe-gamma receptors with low affinity binding include CD32A, CD32B, CD16A, and CD16B. An Fc-epsilon receptor with high affinity binding is FcεRI, and an Fc-epsilon receptor with low affinity binding is FcεRII/CD23.

The binding affinity or binding specificity for an Fe receptor or an ACTR construct comprising an Fc binding domain (e.g., an extracellular ligand-binding domain of an Fe receptor) can be determined by a variety of methods including equilibrium dialysis, equilibrium binding, gel filtration, ELISA, surface plasmon resonance, or spectroscopy.

In some embodiments, the extracellular ligand-binding domain of an Fc receptor comprises an amino acid sequence that is at least 90% (e.g., 91, 92, 93, 94, 95, 96, 97, 98, or 99%) identical to the amino acid sequence of the extracellular ligand-binding domain of a naturally-occurring Fc-gamma receptor, an Fc-alpha receptor, or an Fe-epsilon receptor. The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Also within the scope of the present disclosure are variants of the extracellular ligand-binding domains of Fc receptors, such as those described herein. In some embodiments, the variant extracellular ligand-binding domain may comprise up to 10 amino acid residue variations (e.g., 1, 2, 3, 4, 5, 6, 7, 8, 9, or 10) relative to the amino acid sequence of the reference extracellular ligand-binding domain. In some embodiments, the variant can be a naturally-occurring variant due to one or more gene polymorphisms. In other embodiments, the variant can be a non-naturally occurring modified molecule. For examples, mutations may be introduced into the extracellular ligand-binding domain of an Fc receptor to alter its glycosylation pattern and thus its binding affinity to the corresponding Fc domain.

In some examples, the Fc receptor can be CD16A, CD16B, CD32A, CD32B, CD32C, CD64A, CD64B, CD64C, or a variant thereof as described herein. The extracellular ligand-binding domain of an Fc receptor may comprise up to 10 amino acid residue variations (e.g., 1, 2, 3, 4, 5, 8, 9, or 10) relative to the amino acid sequence of the extracellular ligand-binding domain of CD16A, CD16B, CD32A, CD32B, CD32C, CD64A, CD64B, CD64C as described herein. Such Fc domains comprising one or more amino acid variations may be referred to as a variant. Mutation of amino acid residues of the extracellular ligand-binding domain of an Fc receptor may result in an increase in binding affinity for the Fc receptor domain to bind to an antibody, isotype of antibodies, or subtype(s) thereof relative to Fc receptor domains that do not comprise the mutation. For example, mutation of residue 158 of the Fc-gamma receptor CD16A may result in an increase in binding affinity of the Fc receptor to an Fc portion of an antibody. In some embodiments, the mutation is a substitution of a phenylalanine to a valine at residue 158 of the Fc-gamma receptor CD16A, referred to as a CD16A V158 variant.

The amino acid sequences of human CD16A F158 and CD16A V158 variants are provided below with the F158 and V158 residue highlighted in bold/face and underlined (signal peptide italicized):

CD16A F158 (SEQ ID NO: 70):
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLFGSKNVSSETVNITITQGLAVSTIS
SFFPPGYQ
CD16A V158 (SEQ ID NO: 72):
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQVSFCLVMVLLFAVDTGLYFSVKTNIRSSTRDWKDHKFKWRKD
PQDK

Alternative or additional mutations that can be made in the extracellular ligand-binding domain of an Fc receptor that may enhance or reduce the binding affinity to an Fc portion of a molecule such as an antibody will be evident to one of ordinary skill in the art. In some embodiments, the Fc receptor is CD16A, CD16A V158 variant, CD16A F158 variant, CD16B, CD32A, CD32B, CD32C, CD64A, CD64B, or CD64C.

### B. Transmembrane Domain

The transmembrane domain of the ACTR constructs described herein can be in any form known in the art. As used herein, a “transmembrane domain” refers to any protein structure that is thermodynamically stable in a cell membrane, preferably a eukaryotic cell membrane. Transmembrane domains compatible for use in the ACTR constructs used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the ACTR construct described herein is derived from a Type I single-pass membrane protein. Single-pass membrane proteins include, but are not limited to, CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, PD1, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, PD1, and FGFR2B. In some examples, the transmembrane domain is of CD8α. In some examples, the transmembrane domain is of 4-1BB/CD137. In other examples, the transmembrane domain is of CD28 or CD34. In yet other examples, the transmembrane domain is not derived from human CD8α. In some embodiments, the transmembrane domain of the ACTR construct is a single-pass alpha helix.

Transmembrane domains from multi-pass membrane proteins may also be compatible for use in the ACTR constructs described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the ACTR construct described herein.

Transmembrane domains for use in the ACTR constructs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, or hydrophobic or hydrophilic characteristics of a protein or protein segment, can be assessed by any method known in the art, for example the Kyte and Doolittle hydropathy analysis.

### C. Co-Stimulatory Signaling Domains

Many immune cells require co-stimulation, in addition to stimulation of an antigen-specific signal, to promote cell proliferation, differentiation and survival, as well as to activate effector functions of the cell. In some embodiments, the ACTR constructs described herein comprise at least one co-stimulatory signaling domain. The term “co-stimulatory signaling domain,” as used herein, refers to at least a fragment of a co-stimulatory signaling protein that mediates signal transduction within a cell to induce an immune response such as an effector function. As known in the art, activation of immune cells such as T cells often requires two signals: (1) the antigen specific signal triggered by the engagement of T cell receptor (TCR) and antigenic peptide/MHC complexes presented by antigen presenting cells, which typically is driven by CD3ζ as a component of the TCR complex; and (ii) a co-stimulatory signal triggered by the interaction between a co-stimulatory receptor and its ligand. A co-stimulatory receptor transduces a co-stimulatory signal as an addition to the TCR-triggered signaling and modulates responses mediated by immune cells, such as T cells, NK cells, macrophages, neutrophils, or eosinophils. Any of the ACTR constructs described herein, either containing a co-stimulatory signaling domain or free of such a signaling domain, may be co-expressed in immune cells (e.g., NK cells or T cells) with one or more separate polypeptides comprising a co-stimulatory domain or a ligand of a co-stimulation factor, which provide co-stimulatory signals in trans. Alternatively, the ACTR may be co-expressed in immune cells with a separate polypeptide that provides an immune checkpoint signal.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the ACTR constructs described herein or in the one or more separate polypeptides for co-expression in immune cells described herein. The type(s) of co-stimulatory signaling domain is selected based on factors such as the type of the immune cells in which the ACTR constructs would be expressed (e.g., T cells, NK cells, macrophages, neutrophils, or eosinophils) and the desired immune effector function (e.g., ADCC). Examples of co-stimulatory signaling domains for use in the ACTR constructs or for expression in the immune cells may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the ACTR construct. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant.

In some embodiments, the ACTR constructs may comprise more than one co-stimulatory signaling domain (e.g., 2, 3, or more). In some embodiments, the ACTR construct comprises two or more of the same co-stimulatory signaling domains, for example, two copies of the co-stimulatory signaling domain of CD28. In some embodiments, the ACTR construct comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the ACTR constructs (e.g., T cells or NK cells) and the desired immune effector function. In some embodiments, the ACTR construct comprises two co-stimulatory signaling domains. In some embodiments, the two co-stimulatory signaling domains are CD28 and 4-1BB. In some embodiments, the two co-stimulatory signaling domains are CD28_{LL→GG} variant and 4-1BB. In some embodiments, the two co-stimulatory signaling domains are CD28 and CD27. In some embodiments, the two co-stimulatory signaling domains are CD28 and OX-40. The co-stimulatory domains may occur in any order. For example, the two co-stimulatory domains may occur in order (from 5′ to 3′): CD28 and 4-1BB; CD28_{LL→GG} variant and 4-1BB; CD28 and CD27; or CD28 and OX-40. As another example, the two co-stimulatory domains may occur in order (from 5′ to 3′): 4-1BB and CD28; 4-1BB and CD28_{LL→GG} variant; CD27 and CD28; or OX-40 and CD28.

In some embodiments, the ACTR construct described herein may comprise a single co-stimulatory signaling domain (as the only co-stimulatory signaling domain in an ACTR construct), for example, the co-stimulatory signaling domain from 4-1BB or CD28. In some examples, the ACTR construct may contain only one copy of the single co-stimulatory signaling domain. In other examples, the ACTR construct may contain two copies of the same co-stimulatory signaling domains, for example, two copies of the co-stimulatory signaling domain of CD28.

### D. Cytoplasmic Signaling Domain Comprising an Immunoreceptor Tyrosine-Based Activation Motif (ITAM)

Any cytoplasmic signaling domain comprising an immunoreceptor tyrosine-based activation motif (ITAM) can be used to create the ACTR constructs described herein. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix₍₆₋₈₎YxxL/1. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some examples, the cytoplasmic signaling domain comprising an ITAM is of CD3ζ or FcεR1γ. In other examples, the ITAM-containing cytoplasmic signaling domain is not derived from human CD3ζ. In yet other examples, the ITAM-containing cytoplasmic signaling domain is not derived from an Fc receptor, when the extracellular ligand-binding domain of the same ACTR construct is derived from CD16A.

In one specific embodiment, several signaling domains can be fused together for additive or synergistic effect. Non-limiting examples of useful additional signaling domains include part or all of one or more of TCR Zeta chain and FcεRIγ.

### E. Hinge Domain

In some embodiments, the ACTR constructs described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain of an Fc receptor relative to the transmembrane domain of the ACTR construct can be used.

The hinge domain may contain about 10-100 amino acids, e.g., 15-75 amino acids, 20-50 amino acids, or 30-60 amino acids. In some embodiments, the hinge domain may be of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. In some embodiments, the hinge domain is a hinge domain of a naturally occurring protein. Hinge domains of any protein known in the art to comprise a hinge domain are compatible for use in the ACTR constructs described herein. In some embodiments, the hinge domain is at least a portion of a hinge domain of a naturally occurring protein and confers flexibility to the ACTR construct. In some embodiments, the hinge domain is of CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain is of CD28. In some embodiments, the hinge domain is a portion of the hinge domain of CD28, e.g., a fragment containing at least 15 (e.g., 20, 25, 30, 35, or 40) consecutive amino acids of the hinge domain of CD28.

Hinge domains of antibodies, such as an IgG, IgA, IgM, IgE, or IgD antibodies, are also compatible for use in the ACTR constructs described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the ACTR constructs described herein. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO: 76), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60. In certain embodiments, n can be an integer greater than 60. In some embodiments, the hinge domain is (Gly₄Ser)₃ (SEQ ID NO: 77). In some embodiments, the hinge domain is (Gly₄Ser)₆ (SEQ ID NO: 78). In some embodiments, the hinge domain is (Gly₄Ser)₉ (SEQ ID NO: 79). In some embodiments, the hinge domain is (Gly₄Ser)₁₂ (SEQ ID NO: 80). In some embodiments, the hinge domain is (Gly₄Ser)₁₅ (SEQ ID NO: 81). In some embodiments, the hinge domain is (Gly₄Ser)₃₀ (SEQ ID NO: 82). In some embodiments, the hinge domain is (Gly₄Ser)₄₅ (SEQ ID NO: 83). In some embodiments, the hinge domain is (Gly₄Ser)₆₀ (SEQ ID NO: 84).

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

### F. Signal Peptide

In some embodiments, the ACTR construct also comprises a signal peptide (also known as a signal sequence) at the N-terminus of the polypeptide. In general, signal sequences are peptide sequences that target a polypeptide to the desired site in a cell. In some embodiments, the signal sequence targets the ACTR construct to the secretory pathway of the cell and will allow for integration and anchoring of the ACTR construct into the lipid bilayer. Signal sequences including signal sequences of naturally occurring proteins or synthetic, non-naturally occurring signal sequences that are compatible for use in the ACTR constructs described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16.

### G. Examples of ACTR Constructs

Certain examples of ACTR polypeptides described herein may have, e.g., a CD16A Fc binding domain (e.g., the CD16A F158 variant or the CD16A V158 variant), a CD28 co-stimulatory domain, and a CD3ζ cytoplasmic signaling domain. Such ACTR polypeptides may further comprise a CD28 hinge domain, a CD28 transmembrane domain, or a combination thereof. In some examples, the ACTR polypeptide may further comprise a signal sequence, which may be from CD8α. In one specific example, the ACTR polypeptide comprises, from N-terminus to C-terminus in order: a signal sequence of CD8α, a CD16A Fc binding domain, a CD28 hinge domain, a CD28 transmembrane domain, a CD28 co-stimulatory domain, and a CD3ζ cytoplasmic signaling domain, e.g., SEQ ID NO: 57.

Other examples of ACTR polypeptides described herein may be free of a hinge domain from any non-CD16A receptor (e.g., may have no hinge domain). Such ACTR polypeptides may have, e.g., a CD16A Fc binding domain (e.g., the CD16A F158 variant or the CD16A V158 variant), a CD28 co-stimulatory domain, and a CD3ζ cytoplasmic signaling domain, but have no hinge domain. In some examples, the ACTR polypeptide may additionally comprise a CD8 transmembrane domain. In some examples, the ACTR polypeptide may further comprise a signal sequence, which may be from CD8α. In one specific example, the ACTR polypeptide comprises, from N-terminus to C-terminus in order: a signal sequence of CD8α, a CD16A Fc binding domain, a CD8 transmembrane domain, a CD28 co-stimulatory domain, and a CD3ζ cytoplasmic signaling domain, e.g., SEQ ID NO: 58.

In some embodiments, the ACTR construct described herein may comprise one or more of: an extracellular ligand-binding domain of CD16 (e.g., the CD16A F158 variant or the CD16A V158 variant), hinge and transmembrane domains of CD8α, a co-stimulatory signaling domain of 4-1BB, and a cytoplasmic signaling domain of CD3ζ, e.g., the CD16F-BB-ζ and CD16V-BB-ζ disclosed herein. In certain embodiments, the ACTR construct described herein may comprise an extracellular ligand-binding domain of CD16 (CD16F or CD16V, also known as F158 FCGR3A and V158 FCGR3A variant), hinge and transmembrane domains of CD8α, a co-stimulatory signaling domain of 4-1BB, and a cytoplasmic signaling domain of CD3, e.g., the CD16F-BB-ζ and CD16V-BB-ζ disclosed herein, for example, SEQ ID NO: 1 or SEQ ID NO: 31.

Table 3 provides exemplary ACTR constructs described herein. These exemplary constructs have, from N-terminus to C-terminus in order, the signal sequence, the Fc binding domain (e.g., an extracellular domain of an Fc receptor), the hinge domain, and the transmembrane, while the positions of the optional co-stimulatory domain and the cytoplasmic signaling domain can be switched. In some embodiments, the ACTR polypeptide may comprise any one of SEQ ID NOs: 1-63. In certain embodiments, the ACTR polypeptide may consist of any one of SEQ ID NOs: 1-63. See also WO2015058018A1, US20150139943A1, and PCT/US2018/015999, each of which is incorporated by reference herein for the purpose and subject matter referenced herein.

TABLE 3
Exemplary ACTR constructs.

| Exemplary | | Extracellular | | | | Cytoplasmic |
|---|---|---|---|---|---|---|
| AA Sequence | Signal | domain of Fc | Hinge | Transmembrane | Co-stimulatory | Signaling |
| (SEQ ID NO) | Sequence | receptor | domain | domain | domain | domain |
| 1 | CD8α | CD16A-V158 | CD8α | CD8α | 4-1BB (CD137) | CD3ζ |
| 2 | CD8α | CD16A-V158 | CD8α | 4-1BB (CD137) | 4-1BB (CD137) | CD3ζ |
| 3 | CD8α | CD16A-V158 | CD8α | CD28 | 4-1BB (CD137) | CD3ζ |
| 4 | CD8α | CD16A-V158 | CD8α | CD34 | 4-1BB (CD137) | CD3ζ |
| 5 | CD8α | CD16A-V158 | CD8α | Designed hydrophobic | 4-1BB (CD137) | CD3ζ |
| | | | | TM domain | | |
| 6 | CD8α | CD32A | CD8α | CD8α | 4-1BB (CD137) | CD3ζ |
| 7 | CD8α | CD16A-V158 | CD8α | CD8α | CD28 | CD3ζ |
| 8 | CD8α | CD16A-V158 | CD8α | CD8α | OX40 (CD134) | CD3ζ |
| 9 | CD8α | CD16A-V158 | CD8α | CD8α | CD28 + 4-1BB | CD3ζ |
| 10 | CD8α | CD16A-V158 | None | CD8α | 4-1BB (CD137) | CD3ζ |
| 11 | CD8α | CD16A-V158 | XTEN | CD8α | 4-1BB (CD137) | CD3ζ |
| 12 | CD8α | CD16A-V158 | CD8α | CD8α | CD28 LL to GG mutant | CD3ζ |
| 13 | CD8α | CD16A-V158 | CD8α | CD8α | CD28 LL to GG | CD3ζ |
| | | | | | mutant + 4-1BB | |
| 14 | CD8α | CD16A-V158 | CD8α | CD4 | 4-1BB (CD137) | CD3ζ |
| 15 | CD8α | CD16A-V158 | CD8α | CD4 | CD28 LL to GG | CD3ζ |
| | | | | | mutant + 4-1BB | |
| 16 | CD8α | CD16A-V158 | CD8α | FcεRIγ | 4-1BB (CD137) | CD3ζ |
| 17 | CD8α | CD16A-V158 | CD8α | Designed hydrophobic | 4-1BB (CD137) | CD3ζ |
| | | | | TM domain, predicted | | |
| | | | | dimerization | | |
| 18 | CD8α | CD16A-V158 | CD8α | CD8β | 4-1BB (CD137) | CD3ζ |
| 19 | CD8α | CD16A-V158 | CD8α | C16α | 4-1BB (CD137) | CD3ζ |
| 20 | CD8α | CD16A-V158 | CD8α | OX40 (CD134) | 4-1BB (CD137) | CD3ζ |
| 21 | CD8α | CD16A-V158 | CD8α | CD3ζ | 4-1BB (CD137) | CD3ζ |
| 22 | CD8α | CD16A-V158 | CD8α | CD3ε | 4-1BB (CD137) | CD3ζ |
| 23 | CD8α | CD16A-V158 | CD8α | CD3γ | 4-1BB (CD137) | CD3ζ |
| 24 | CD8α | CD16A-V158 | CD8α | CD3δ | 4-1BB (CD137) | CD3ζ |
| 25 | CD8α | CD16A-V158 | CD8α | TCR-α | 4-1BB (CD137) | CD3ζ |
| 26 | CD8α | CD16A-V158 | CD8α | CD32 | 4-1BB (CD137) | CD3ζ |
| 27 | CD8α | CD16A-V158 | CD8α | CD64 | 4-1BB (CD137) | CD3ζ |
| 28 | CD8α | CD16A-V158 | CD8α | VEGFR2 | 4-1BB (CD137) | CD3ζ |
| 29 | CD8α | CD16A-V158 | CD8α | FAS | 4-1BB (CD137) | CD3ζ |
| 30 | CD8α | CD16A-V158 | CD8α | FGFR2B | 4-1BB (CD137) | CD3ζ |
| 31 | CD8α | CD16A-F158 | CD8α | CD8α | 4-1BB (CD137) | CD3ζ |
| 32 | CD8α | CD64A | CD8α | CD8α | 4-1BB (CD137) | CD3ζ |
| 33 | CD8α | CD16A-V158 | IgG1 (hinge-CH2-CH3) | CD8α | 4-1BB (CD137) | CD3ζ |
| 34 | CD8α | CD16A-V158 | IgG1 (hinge-CH3) | CD8α | 4-1BB (CD137) | CD3ζ |
| 35 | CD8α | CD16A-V158 | IgG1 (hinge) | CD8α | 4-1BB (CD137) | CD3ζ |
| 36 | CD8α | CD16A-V158 | CD8-alpha fragment | CD8α | 4-1BB (CD137) | CD3ζ |
| | | | 1 (30 amino acids) | | | |
| 37 | CD8α | CD16A-V158 | CD8-alpha fragment | CD8α | 4-1BB (CD137) | CD3ζ |
| | | | 2 (15 amino acids) | | | |
| 38 | CD8α | CD16A-V158 | (Gly4Ser) | CD8α | 4-1BB (CD137) | CD3ζ |
| | | | x3 (60 amino acids) | | | |
| 39 | CD8α | CD16A-V158 | (Gly4Ser) | CD8α | 4-1BB (CD137) | CD3ζ |
| | | | x6 (45 amino acids) | | | |
| 40 | CD8α | CD16A-V158 | (Gly4Ser) | CD8α | 4-1BB (CD137) | CD3ζ |
| | | | x9 (30 amino acids) | | | |
| 41 | CD8α | CD16A-V158 | (Gly4Ser) | CD8α | 4-1BB (CD137) | CD3ζ |
| | | | x12 (15 amino acids) | | | |
| 42 | CD8α | CD16A-V158 | XTEN (60 amino acids) | CD8α | 4-1BB (CD137) | CD3ζ |
| 43 | CD8α | CD16A-V158 | XTEN (30 amino acids) | CD8α | 4-1BB (CD137) | CD3ζ |
| 44 | CD8α | CD16A-V158 | XTEN (15 amino acids) | CD8α | 4-1BB (CD137) | CD3ζ |
| 45 | CD28 | CD16A-V158 | CD8α | CD8α | 4-1BB (CD137) | CD3ζ |
| 46 | Murine kappa chain | CD16A-V158 | CD8α | CD8α | 4-1BB (CD137) | CD3ζ |
| 47 | CD16 | CD16A-V158 | CD8α | CD8α | 4-1BB (CD137) | CD3ζ |
| 48 | CD8α | CD16A-V158 | CD8α | CD8α | ICOS | CD3ζ |
| 49 | CD8α | CD16A-V158 | CD8α | CD8α | CD27 | CD3ζ |
| 50 | CD8α | CD16A-V158 | CD8α | CD8α | GITR | CD3ζ |
| 51 | CD8α | CD16A-V158 | CD8α | CD8α | HVEM | CD3ζ |
| 52 | CD8α | CD16A-V158 | CD8α | CD8α | TIM1 | CD3ζ |
| 53 | CD8α | CD16A-V158 | CD8α | CD8α | LFA1 (CD11a) | CD3ζ |
| 54 | CD8α | CD16A-V158 | CD8α | CD8α | CD2 | CD3ζ |
| 55 | CD8α | CD16A-V158 | CD8α | FcεR1γ | 4-1BB (CD137) | FcεR1γ |
| 56 | CD8α | CD16A-V158 | CD8α | CD8α | 4-1BB (CD137) | FcεR1γ |
| 57 | CD8α | CD16A-V158 | CD28 | CD28 | CD28 | CD3ζ |
| 58 | CD8α | CD16A-V158 | none | CD8 | CD28 | CD3ζ |
| 59 | CD8α | CD16A-V158 | CD8 | CD8 | CD28 + CD27 | CD3ζ |
| 60 | CD8α | CD16A-V158 | CD8 | CD8 | CD28 + OX40 | CD3ζ |
| 61 | CD8α | CD16A-V158 | CD8 | CD8 | 4-1BB + CD28 | CD3ζ |
| 62 | CD8α | CD16A-V158 | CD28 | CD28 | CD28 + 4-1BB | CD3ζ |
| 63 | CD8α | CD16A-V158 | CD28 | CD28 | 4-1BB | CD3ζ |

Amino acid sequences of the example ACTR constructs are provided below (signal sequence italicized).

SEQ ID NO: 1:
MALPVTALLLPLALLLHAARPGMRTEDLPKAVVFLEPQWYRVLEKDSVTLKCQGAYSPEDNSTQWFHNESLISSQ
ASSYFIDAATVDDSGEYRCQTNLSTLSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLAVSTISSFFPPGYQTTTPAPRPPTPA
PTIASQPLSLRPEACRPAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 2:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIISFFLALTSTALLFLLFFLTLRFSVVKRG
KRGRKKLLYIFKQPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 3:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDFWVLVVVGGVLACYSLLVTVAFIIFWVRSK
KRGRKKLLYIFKQPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 4:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLIALVTSGALLAVLGITGYFLMNRKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 5:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLLAALLALLAALLALLAALLARSKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 6:
MALPVTALLLPLALLLHAARPQAAAPPKAVLKLEPPWINVLQEDSVTLTCQGARSPESDSIQWFHNGNLIPT
HTQPSYRFKANNNDSGEYTCQTGQTSLSDPVHLTVLSEWLVLQTPHLEFQEGETIMLRCHSWKDKPLVKVTF
FQNGKSQKFSHLDPTFSIPQANHSHSGDYHCTGNIGYTLFSSKPVTITVQVPSMGSSSPMGTTTPAPRPPTP
APTIASQPLSLRPEACRPAAGGAVHTRGLDFACDIYIWAPLAGTCGVLLLSLVITLYCKRGRKKLLYIFKQP
FMRPVQTTQEEDGCSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR
SEQ ID NO: 7:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 8:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCALYLLR
RDQRLPPDAHKPPGGGSFRTPIQEEQADAHSTLAKIRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 9:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 10:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 11:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTSESATPESGPGTSTEPSEGSAPGSPAGSPTIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 12:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
GGHSDYMNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 13:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
GGHSDYMNMTPRRPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 14:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDMALIVLGGVAGLLLFIGLGIFFCVRKRGRK
KLLYIFKQPFMRPVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSTATKDTYDALHMQ
ALPPR
SEQ ID NO: 15:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDMALIVLGGVAGLLLFIGLGIFFCVRRSKRS
RGGHSDYMNMTPRRPGPTRKHYQPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 16:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLCYILDAILFLYGIVLTLLYCRLKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 17:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLLLILLGVLAGVLATLAALLARSKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 18:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDITLGLLVAGVLVLLVSLGVAIHLCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 19:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVSFCLVMVLLFAVDTGLYFSVKTNKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 20:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVAAILGLGLVLGLLGPLAILLALYKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 21:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLCYLLDGILFIYGVILTALFLRVKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 22:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVMSVATIVIVDICITGGLLLLVYYWSKNRK
RGRKKLLYIFKQPFMRPVQTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKGHDGLYQGLSTATKDTYDA
LHMQALPPR
SEQ ID NO: 23:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDGFLFAEIVSIFVLAVGVYFIAGQDKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 24:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDGIIVTDVIATLLLALGVFCFAGHETKRGRK
KLLYIFKQPFMRPVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSTATKDTYDALHMQ
ALPPR
SEQ ID NO: 25:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVIGFRILLLKVAGFNLLMTLRLWKRGRKKL
LYIFKQPFMRPVQTTQEEDGCSCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 26:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIIVAVVIATAVAAIVAAVVALIYCRKKRGR
KKLLYIFKQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 27:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVLFYLAVGIMFLVNTVLWVTIRKEKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 28:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIIILVGTAVIAMFFWLLLVIILRTKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 29:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLGWLCLLLLPIPLIVWVKRKKRGRKKLLYI
FKQPFMRPVQTTQEEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPR
SEQ ID NO: 30:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIAIYCIGVFLIACMVVTVILCRMKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 31:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLFGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 32:
MALPVTALLLPLALLLHAARPQVDTTKAVITLQPPWVSVFQEETVTLHCEVLHLPGSSSTQWFLNGTATQTS
TPSYRITSASVNDSGEYRCQRGLSGRSDPIQLEIHRGWLLLQVSSRVFTEGEPLALRCHAWKDKLVYNVLYY
RNGKAFKFFHWNSNLTILKTNISHNGTYHCSGMGKHRYTSAGISVTVKELFPAPVLNASVTSPLLEGNLVTL
SCETKLLLQRPGLQLYFSFYMGSKTLRGRNTSSEYQILTARREDSGLYWCEAATEDGNVLKRSPELELQVLG
LQLPTPVWFHIYIWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID NO: 33:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQEPK
SCDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCSVMHEALHN
HYTQKSLSLSPGKIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 34:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQEPK
SCDKTHTCPGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGKIYIWAPLAGTCGVLLLSLVITLYCKRGR
KKLLYIFKQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 35:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQEPK
SCDKTHTCPIYIWAPLAGTCGVLLLSLVITLYCKRGRKKLLYIFKQPFMRPVQTTQEEDGCSCRFPEEEEGG
CELRVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 36:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEAFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 37:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPFACDIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 38:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 39:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSGGGGSGGGGSGGGGSIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 40:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSGGGGSGGGGSGGGGSGGGGSGGGGSGGGGSIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 41:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSGGGGSGGGGSGGGGSGGGGSGGGGSGGGGSGGGGSGGGGSGGGGSIYIWAPLAGTCGVLL
LSLVITLYCKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGLYQGL
STATKDTYDALHMQALPPR
SEQ ID NO: 42:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTSESATPESGPGTSTEPSEGSAPGSPAGSPTSTEEGTSTEPSEGSAIYIWAPLAGTCGVLL
LSLVITLYCKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGLYQGL
STATKDTYDALHMQALPPR
SEQ ID NO: 43:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTSESATPESGPGTSTEIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 44:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 45:
MLRLLLALNLFPSIQVTGGMRTEDLPKAVVFLEPQWYRVLEKDSVTLKCQGAYSPEDNSTQWFHNESLISSQ
ASSYFIDAATVDDSGEYRCQTNLSTLSDPVQLEVHIGWLLLQAPRWVFKEEDPIHLRCHSWKNTALHKVTYL
QNGKGRKYFHHNSDFYIPKATLKDSGSYFCRGLVGSKNVSSETVNITITQGLAVSTISSFFPPGYQTTTPAP
RPPTPAPTIASQPLSLRPEACRPAAGGAVHTRGLDFACDIYIWAPLAGTCGVLLLSLVITLYCKRGRKKLLY
IFKQPFMRPVQTTQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDTYDALHMQALPP
R
SEQ ID NO: 46:
METDTLLLWVLLLWVPGSTGDGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 47:
MWQLLLPTALLLLVSAGMRTEDLPKAVVFLEPQWYRVLEKDSVTLKCQGAYSPEDNSTQWFHNESLISSQAS
SYFIDAATVDDSGEYRCQTNLSTLSDPVQLEVHIGWLLLQAPRWVFKEEDPIHLRCHSWKNTALHKVTYLQN
GKGRKYFHHNSDFYIPKATLKDSGSYFCRGLVGSKNVSSETVNITITQGLAVSTISSFFPPGYQTTTPAPRP
PTPAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPR
SEQ ID NO: 48:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCCWLTKK
KYSSSVHDPNGEYMFMRAVNTAKKSRLTDVTLRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPR
SEQ ID NO: 49:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCQRRKYR
SNKGESPVEPAEPCRYSCPREEEGSTIPIQEDYRKPEPACSPRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 50:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCQLGLHI
WQLRSQCMWPRETQLLLEVPPSTEDARSCQFPEEERGERSAEEKGRLGDLWVRVKFSRSADAPAYQQGQNQL
YNELNLGRREEYDVLDKRRGRDPEMGGKPRRKNPQEGLYNELQKDKMAEAYSEIGMKGERRRGKGHDGLYQG
LSTATKDTYDALHMQALPPR
SEQ ID NO: 51:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCCVKRRK
PRGDVVKVIVSVQRKRQEAEGEATVIEALQAPPDVTTVAVEETIPSFTGRSPNHRVKFSRSADAPAYQQGQN
QLYNELNLGRREEYDVLDKRRGRDPEMGGKPRRKNPQEGLYNELQKDKMAEAYSEIGMKGERRRGKGHDGLY
QGLSTATKDTYDALHMQALPPR
SEQ ID NO: 52:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKKYFFK
KEVQQLSVSFSSLQIKALQNAVEKEVQAEDNIYIENSLYATDRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 53:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCYKVGFF
KRNLKEKMEAGRGVPNGIPAEDSEQLASGQEAGDPGCLKPLHEKDSESGGGKDRVKFSRSADAPAYQQGQNQ
LYNELNLGRREEYDVLDKRRGRDPEMGGKPRRKNPQEGLYNELQKDKMAEAYSEIGMKGERRRGKGHDGLYQ
GLSTATKDTYDALHMQALPPR
SEQ ID NO: 54:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRKKQR
SRRNDEELETRAHRVATEERGRKPHQIPASTPQNPATSQHPPPPPGHRSQAPSHRPPPPGHRVQHQPQKRPP
APSGTQVHQQKGPPLPRPRVQPKPPHGAAENSLSPSSNRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 55:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDPQLCYILDAILFLYGIVLTLLYCRLKIQVR
KAAITSYEKSDGVYTGLSTRNQETYETLKHEKPPQKRGRKKLLYIFKQPFMRPVQTTQEEDGCSCRFPEEEE
GGCEL
SEQ ID NO: 56:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEFDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRLKIQVRKAAITSYEKSDGVYTGLSTRNQETYETLK
HEKPPQ
SEQ ID NO: 57:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPFWVLVVVGGVLACYSLLVTVAFIIFWVRSKRSRLLH
SDYMNMTPRRPGPTRKHYQPYAPPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPR
SEQ ID NO: 58:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCRSKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR
SEQ ID NO: 59:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEFDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSQRRKYRSNKGESPVEPAEPCHYSCPREEEGSTIPIQE
DYRKPEPACSPRVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 60:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSRRDQRLPPDAHKPPGGGSFRTPIQEEQADAHSTLAKI
RVKFSRSADAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID NO: 61:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRSKRSRLLHSDYMNMTPRRPGPTRKHYQPYAPPRDF
AAYRSRVKFSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 62:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPFWVLVVVGGVLACYSLLVTVAFIIFWVRSKRSRLLH
SDYMNMTPRRPGPTRKHYQPYAPPRDFAAYRSKRGRKKLLYIFKQPFMRPVQTTQEEDGCSCRFPEEEEGGC
ELRVKFSRSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 63:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPFWVLVVVGGVLACYSLLVTVAFIIFWVKRGRKKLLY
IFKQPFMRPVQTTQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDTYDALHMQA
LPPR

In some examples, the ACTR constructs described herein comprise one or more of the below-listed components. In some examples, the ACTR constructs described herein do not include all of the below-listed components. For example, the ACTR construct described herein may not comprise one or more of the sequences listed in Table 4 below.

TABLE 4
Exemplary sequences

| SEQ | | |
|---|---|---|
| ID NO | SEQUENCE | DESCRIPTION |
| 64 | TTTPAPRPPTPAPTIASQPLSLRPEACRPAA | hinge and |
| | GGAVHTRGLDFACDIYIWAPLAGTCGVLLLS | transmembrane |
| | LVITLYC | domains of |
| | | CD8alpha |
| 65 | KRGRKKLLYIFKQPFMRPVQTTQEEDGCSCR | 4-1BB |
| | FPEEEEGGCEL | signaling |
| | | domain |
| 66 | RVKFSRSADAPAYQQGQNQLYNELNLGRRE | CD3zeta |
| | EYDVLDKRRGRDPEMGGKPRRKNPQEGLYNE | signaling |
| | LQKDKMAEAYSEIGMKGERRRGKGHDGLYQG | domain |
| | LSTATKDTYDALHMQALPPR | |
| 67 | MALPVTALLLPLALLLHAARP | signal |
| | | peptide of |
| | | CD8alpha |

Like other ACTR constructs disclosed herein, expression of these exemplary ACTR constructs in immune cells such as T cells and NK cells would confer ADCC capability to these cells and, therefore, would significantly augment the anti-tumor potential of anti-BCMA antibodies.

### H. Preparation of and Pharmaceutical Compositions Comprising ACTR Constructs

Any of the ACTR constructs described herein can be prepared by a routine method, such as recombinant technology. Methods for preparing the ACTR constructs herein involve generation of a nucleic acid that encodes a polypeptide comprising each of the domains of the ACTR constructs, including the extracellular ligand-binding domain of an Fc receptor, the transmembrane domain, and the cytoplasmic signaling domain comprising an ITAM. The nucleic acid construct may also include one or more co-stimulatory signaling domains. In some embodiments, the nucleic acid also encodes a hinge domain between the extracellular ligand-binding domain of an Fc receptor and the transmembrane domain. The nucleic acid encoding the ACTR construct may also encode a signal sequence. In some embodiments, the nucleic acid sequence encodes any one of the exemplary ACTR constructs provided by SEQ ID NO: 1-63.

Sequences of each of the components of the ACTR constructs may be obtained via routine technology, e.g., PCR amplification from any one of a variety of sources known in the art. In some embodiments, sequences of one or more of the components of the ACTR constructs are obtained from a human cell. Alternatively, the sequences of one or more components of the ACTR constructs can be synthesized. Sequences of each of the components (e.g., domains) can be joined directly or indirectly (e.g., using a nucleic acid sequence encoding a peptide linker) to form a nucleic acid sequence encoding the ACTR construct, using methods such as PCR amplification or ligation. Alternatively, the nucleic acid encoding the ACTR construct may be synthesized. In some embodiments, the nucleic acid is DNA. In other embodiments, the nucleic acid is RNA.

### II. Immune Cells Expressing ACTR Constructs

Host cells expressing the ACTR constructs (ACTR-expressing cells, e.g., ACTR T cells) described herein provide a specific population of cells that can recognize BCMA-positive target cells bound by Fc-containing anti-BCMA antibodies. In one embodiment, engagement of the extracellular ligand-binding domain of an ACTR construct expressed on such host cells with the Fc portion of an anti-BCMA antibody transmits an activation signal to the optional co-stimulatory signaling domain(s) and/or the ITAM-containing cytoplasmic signaling domain of the ACTR construct, which in turn activates cell proliferation and/or effector functions of the host cell, such as ADCC effects triggered by the host cells. In another embodiment, engagement of the extracellular Fc-binding domain of an ACTR construct expressed on such host cells with the Fc portion of an anti-BCMA antibody transmits an activation signal to the ITAM-containing cytoplasmic signaling domain of the ACTR construct and/or the one or more co-stimulatory signaling domains co-expressed in such host cells, which in turn activates cell proliferation and/or effector functions of the host cell, such as ADCC effects triggered by the host cells. The combination of co-stimulatory signaling domain(s) and the cytoplasmic signaling domain comprising an ITAM may allow for robust activation of multiple signaling pathways within the cell. In some embodiments, the host cells are immune cells, such as T cells or NK cells. In some embodiments, the immune cells are T cells. In some embodiments, the immune cells are NK cells. In other embodiments, the immune cells can be established cell lines, for example, NK-92 cells.

Any of the ACTR constructs described herein may be co-expressed in the immune cells with the one or more separate polypeptides described herein for providing co-stimulatory signals in trans.

The population of immune cells can be obtained from any source, such as peripheral blood mononuclear cells (PBMCs), bone marrow, or tissues such as spleen, lymph node, thymus, or tumor tissue. A source suitable for obtaining the type of host cells desired would be evident to one of skill in the art. In some embodiments, the population of immune cells is derived from PBMCs. The type of host cells desired (e.g., T cells, NK cells, or T cells and NK cells) may be expanded within the population of cells obtained by co-incubating the cells with stimulatory molecules. As a non-limiting example, anti-CD3 and anti-CD28 antibodies may be used for expansion of T cells.

To construct the immune cells that express any of the ACTR constructs described herein, expression vectors for stable or transient expression of the ACTR construct may be created via conventional methods as described herein and introduced into immune host cells. For example, nucleic acids encoding the ACTR constructs may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the ACTR constructs. The synthetic linkers may contain nucleic acid sequences that correspond to a particular restriction site in the vector. The selection of expression vectors/plasmids/viral vectors would depend on the type of host cells for expression of the ACTR constructs, but should be suitable for integration and replication in eukaryotic cells.

A variety of promoters can be used for expression of the ACTR constructs described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, or herpes simplex tk virus promoter. Additional promoters for expression of the ACTR constructs include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the ACTR construct.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, nitroreductase, and caspases such as caspase 8.

Suitable vectors and methods for producing vectors containing transgenes are well known and available in the art. Examples of the preparation of vectors for expression of ACTR constructs can be found, for example, in US2014/0106449, herein incorporated in its entirety by reference.

Any of the vectors comprising a nucleic acid sequence that encodes an ACTR construct described herein is also within the scope of the present disclosure. Such a vector, or the sequence encoding an ACTR construct contained therein, may be delivered into host cells such as host immune cells by any suitable method. Methods of delivering vectors to immune cells are well known in the art and may include DNA electroporation, RNA electroporation, transfection using reagents such as liposomes, or viral transduction.

In some embodiments, the vectors for expression of the ACTR constructs are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; and WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655). In some embodiments, the vectors for expression of the ACTR constructs are retroviruses. In some embodiments, the vectors for expression of the ACTR constructs are lentiviruses.

Examples of references describing retroviral transduction include Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993). International Patent Publication No. WO 95/07358 describes high efficiency transduction of primary B lymphocytes. See also WO2016040441A1, which is incorporated by reference herein for the purpose and subject matter referenced herein.

In examples in which the vectors encoding ACTR constructs are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

In some embodiments, RNA molecules encoding any of the ACTR constructs as described herein may be prepared by a conventional method (e.g., in vitro transcription) and then introduced into suitable host cells, e.g., those described herein, via known methods, e.g., Rabinovich et al., Human Gene Therapy 17:1027-1035.

Following introduction into the host cells a vector encoding any of the ACTR constructs provided herein, or the nucleic acid encoding a chimeric vector (e.g., an RNA molecule), the cells are cultured under conditions that allow for expression of the ACTR construct. In examples in which the nucleic acid encoding the ACTR construct is regulated by a regulatable promoter, the host cells are cultured in conditions wherein the regulatable promoter is activated. In some embodiments, the promoter is an inducible promoter and the immune cells are cultured in the presence of the inducing molecule or in conditions in which the inducing molecule is produced. Determining whether the ACTR construct is expressed will be evident to one of skill in the art and may be assessed by any known method, for example, detection of the ACTR construct-encoding mRNA by quantitative reverse transcriptase PCR (qRT-PCR) or detection of the ACTR construct protein by methods including Western blotting, fluorescence microscopy, and flow cytometry. Alternatively, expression of the ACTR construct may take place in vivo after the immune cells are administered to a subject.

As used herein, the term “subject” refers to any mammal. For example, the subject may be a primate. In a preferred embodiment, the subject is human.

Alternatively, expression of an ACTR construct in any of the immune cells disclosed herein can be achieved by introducing RNA molecules encoding the ACTR constructs. Such RNA molecules can be prepared by in vitro transcription or by chemical synthesis. The RNA molecules can then introduced into suitable host cells such as immune cells (e.g., T cells, NK cells, or both T cells and NK cells) by, e.g., electroporation. For example, RNA molecules can be synthesized and introduced into host immune cells following the methods described in Rabinovich et al., Human Gene Therapy, 17:1027-1035 and WO WO2013/040557.

Methods for preparing host cells expressing any of the ACTR constructs described herein may also comprise activating the host cells ex vivo. Activating a host cell means stimulating a host cell into an activate state in which the cell may be able to perform effector functions (e.g., ADCC). Methods of activating a host cell-will depend on the type of host cell used for expression of the ACTR constructs. For example, T cells may be activated ex vivo in the presence of one or more molecules including, but not limited to: an anti-CD3 antibody, an anti-CD28 antibody, IL-2, and/or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and/or K562 cells. In some embodiments, the host cells expressing any of the ACTR constructs (ACTR-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

The methods of preparing host cells expressing any of the ACTR constructs described herein may comprise expanding the host cells ex vivo. Expanding host cells may involve any method that results in an increase in the number of cells expressing ACTR constructs, for example, allowing the host cells to proliferate or stimulating the host cells to proliferate. Methods for stimulating expansion of host cells will depend on the type of host cell used for expression of the ACTR constructs and will be evident to one of skill in the art. In some embodiments, the host cells expressing any of the ACTR constructs described herein are expanded ex vivo prior to administration to a subject.

In some embodiments, the host cells expressing the ACTR construct are expanded and activated ex vivo prior to administration of the cells to the subject. Host cell activation and expansion may be used to allow integration of a viral vector into the genome and expression of the gene encoding an ACTR construct as described herein. If mRNA electroporation is used, no activation and/or expansion may be required, although electroporation may be more effective when performed on activated cells. In some instances, an ACTR construct is transiently expressed in a suitable host cell (e.g., for 3-5 days). Transient expression may be advantageous if there is a potential toxicity and should be helpful in initial phases of clinical testing for possible side effects.

Any of the host cells expressing the ACTR construct may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated and/or for the enhancement of ADCC, preferably those with complementary activities that do not adversely affect each other. Non-limiting examples of possible additional active compounds include, e.g., IL2 as well as various agents known in the field and listed in the discussion of combination treatments, below.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

### III. Anti-BCMA Antibodies

B-cell maturation antigen (BCMA) is a cell surface receptor of the TNF receptor superfamily. It is expressed in B cells (highly expressed in mature B lymphocytes) and plasma cells and has been shown to specifically bind to BAFF (B-cell activating factor), leading to NF-kappaB and MAPK8/JNK activation. See, e.g., Bayles and Milcarek, Crit Rev Immunol. 2014; 34(6):481-99; Coquery and Erickson, Crit Rev Immunol. 2012; 32(4):287-305; and Rickert et al., Immunol Rev. 2011 November; 244(1):115-33.

The sequence of an exemplary wild-type human BCMA (also provided by GenBank under accession number BAB60895) is provided below.

(SEQ ID NO: 71)

MLQMAGQCSQNEYFDSLLHACIPCQLRCSSNTPPLTCQRYCNASVTNSV
KGTNAILWTCLGLSLIISLAVFVLMFLLRKISSEPLKDEFKNTGSGLLG
MANIDLEKSRTGDEIILPRGLEYTVEECTCEDCIKSKPKVDSDHCFPLP
AMEEGATILVTTKTNDYCKSLPAALSATEIEKSISAR

(i) Anti-BCMA Antibodies

The present disclosure relates to the co-use of ACTR T cells and anti-BCMA antibodies in immunotherapy (e.g., cancer immunotherapy). An anti-BCMA antibody refers to an antibody capable of binding to a BCMA protein, for example, a BCMA protein expressed on cell surface.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof which comprise an Fc region, mutants thereof, fusion proteins comprising an antibody portion, humanized antibodies, chimeric antibodies, diabodies, nanobodies, linear antibodies, multispecific antibodies (e.g., bispecific antibodies) and any other modified configuration of the immunoglobulin molecule that comprises an antigen recognition site of the required specificity and an Fc region, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The anti-BCMA antibody for use in the present disclosure contains an Fc region recognizable by the co-used ACTR T cells. The Fc region may be a human or humanized Fc region.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the anti-BCMA antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (BCMA) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen. In some embodiments, the antibodies described herein specifically bind to BCMA.

In some embodiments, an anti-BCMA antibody as described herein has a suitable binding affinity for the target antigen (e.g., BCMA) or antigenic epitopes thereof. As used herein, “binding affinity” refers to the apparent association constant or K_{A}. The K_{A} is the reciprocal of the dissociation constant (K_{D}). The anti-BCMA antibody described herein may have a binding affinity (K_{D}) of at least 10⁻⁵, 10⁻⁶, 10⁻⁷, 10⁻⁸, 10⁻⁹, 10⁻¹⁰M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences In binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the anti-BCMA antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The anti-BCMA antibodies for use in the immune therapy methods described herein may bind to (e.g., specifically bind to) a specific region or an antigenic epitope therein.

Exemplary anti-BCMA antibodies for use with the methods described herein include antibodies described herein as well as other known antibodies. The sequences of certain exemplary anti-BCMA antibody are provided in Table 5 below (Kabat CDR sequences underlined in VH and VL sequences).

TABLE 5
Exemplary BCMA antibody sequences
(VH and VL CDRs underlined).

| Exemplary AA | | |
|---|---|---|
| Sequence | | |
| (SEQ ID NO) | Description | Sequence |
| 68 | Rat SG16.17 VH | QVNLLQSRAALVKPGASVKLSCEASGYT |
| | | FTDYYIIIWVKQSHGKSLEWIGYINPNSG |
| | | YTKYNENFKTKATMTADKSTNTAYVELS |
| | | RLTSEDSATYFCTRYMWERVTGFFDFWG |
| | | PGTKVTVSS |
| 69 | hSG16.17 VH1 | QVQLVQSRAEVKKPGASVKLSCKASGYT |
| | | FTDYYIHWVRQAPGQGLEWIGYINPNSG |
| | | YTKYNENFKTRATMTADKSINTAYVELS |
| | | RLRSDDTAVYFCTRYMWERVTGFFDFW |
| | | GQGTMVTVSS |
| 73 | hSG16.17 VH2 | QVQLVQSGAEVKKPGASVKLSCKASGYT |
| | | FTDYYMHWVRQAPGQGLEWIGRINPNSG |
| | | YTNYAQKFQGRVTMTADKSINTAYVELS |
| | | RLRSDDTAVYFCTRYMWERVTGFFDFW |
| | | GQGTMVTVSS |
| 74 | hSG16.17 VH3 | QVQLVQSGAEVKKPGASVKLSCKASGYT |
| | | FTDYYIHWVRQAPGQGLEWIGYINPNSG |
| | | YTNYAQKFQGRATMTADKSINTAYVELS |
| | | RLRSDDTAVYFCTRYMWERVTGFFDFW |
| | | GQGTMVTVSS |
| 75 | hSG16.17 VH4 | QVQLVQSGAEVKKPGASVKVSCKASGY |
| | | TFTDYYMHWVRQAPGQGLEWIGRINPNS |
| | | GYTNYAQKFQGRATMTADKSINTAYVEL |
| | | SRLRSDDTAVYFCTRYMWERVTGFFDF |
| | | WGQGTMVTVSS |
| 88 | hSG16.17 VH5 | QVQLVQSGAEVKKPGASVKVSCKASGY |
| | | TFTDYYIHWVRQAPGQGLEWIGYINPNS |
| | | GYTKYNENFKTRATMTADKSTNTAYVEL |
| | | SSLRSEDTAVYFCTRYMWERVTGFFDFW |
| | | GQGTMVTVSS |
| 89 | hSG16.17 VH6 | QVQLVQSRAEVKKPGASVKLSCKASGYT |
| | | FTDYYMHWVRQAPGQGLEWIGIINPNSG |
| | | YTSYAQKFQGRVTMTADKSTNTAYVELS |
| | | SLRSEDTAVYFCTRYMWERVTGFFDFWG |
| | | QGTMVTVSS |
| 90 | Rat SG16.17 VL | DIQMTQSPASLSASLGETVSIECLASEDIS |
| | | DDLAWYQQKSGKSPQVLVYTTSRLQDG |
| | | VPSRFSGSGSGTRFSLKIIVMQPEDEADYF |
| | | CQQTYKFPPTFGAGTRLDLKR |
| 91 | hSG16.17 VK2 | DIQMTQSPSSVSASVGDRVTITCLASEDIS |
| | | DDLAWYQQKPGKAPKVLVYTTSSLQSG |
| | | VPSRFSGSGSGTDFTLTISSLQPEDFATYF |
| | | CQQTYKFPPTFGGGTKVEIKR |
| 92 | hSG16.17 VK3 | DIQMTQSPSSVSASVGDRVTITCRASEDIS |
| | | DDLAWYQQKPGKAPKVLVYTTSSLQSG |
| | | VPSRFSGSGSGTDFTLTISSLQPEDFATYF |
| | | CQQTYKFPPTFGGGTKVEIKR |
| 93 | hSG16.17 VK4 | DIQMTQSPSSVSASVGDRVTITCLASEDIS |
| | | DDLAWYQQKPGKAPKVLVYTTSRLQSG |
| | | VPSRFSGSGSGTDFTLTISSMQPEDFADYF |
| | | CQQTYKFPPTFGGGTKVEIKR |
| 94 | hSG16.17 VK5 | DIQMTQSPSSVSASVGDRVTITCRASEDIS |
| | | DDLAWYQQKSGKAPKVLVYTTSSLQSG |
| | | VPSRFSGSGSGTDFTLTISSLQPEDFATYF |
| | | CQQTYKFPPTFGGGTKVEIKR |
| 95 | Rat SG16.45 VH | EVQLVESGGGLVQPGRSLKLSCVASGFTF |
| | | NDHWMTWIRQAPGRGLEWISSITNTGGA |
| | | TYYLDSVKGRFTISRDNAKSTLYLQMNS |
| | | LRSEDTATYYCTSPGLYFDYWGQGVMV |
| | | TVSS |
| 96 | hSG16.45 VH1 | EVQLLESGGGLVQPGGSLRLSCAASGFTF |
| | | NDHWMTWTRQAPGKGLEWISSITNTGGA |
| | | TYYLDSVKGRFTISRDNSKNTLYLQMNS |
| | | LRAEDTAVYYCTSPGLYFDYWGQGVMV |
| | | TVSS |
| 97 | hSG16.45 VH2 | EVQLLESGGGLVQPGGSLRLSCAASGFTF |
| | | NDHWMTWIRQAPGKGLEWISAITNTGGA |
| | | TYYADSVKGRFTISRDNSKNTLYLQMNS |
| | | LRAEDTAVYYCTSPGLYFDYWGQGVMV |
| | | TVSS |
| 98 | hSG16.45 VH3 | EVQLLESGGGLVQPGGSLRLSCAASGFTF |
| | | NDHWMTWIRQAPGKGLEWISAITNTGGA |
| | | TYYADSVKGRFTISRDNSKSTLYLQMNSL |
| | | RAEDTAVYYCTSPGLYFDYWGQGVMVT |
| | | VSS |
| 99 | hSG16.45 VH4 | EVQLLESGGGLVQPGGSLRLSCAASGFTF |
| | | NDHWMTWVRQAPGKGLEWISSITNTGG |
| | | ATYYADSVKGRFTISRDNSKSTLYLQMN |
| | | SLRAEDTAVYYCTSPGLYFDYWGQGTM |
| | | VTVSS |
| 100 | hSG16.45 VH5 | EVQLVESGGGLVQPGGSLRLSCAASGFTF |
| | | NDHWMTWVRQAPGKGLVWVSSITNTGG |
| | | ATYYADSVKGRFTISRDNAKNTLYLQMN |
| | | SLRAEDTAVYYCTSPGLYFDYWGQGTM |
| | | VTVSS |
| 101 | hSG16.45 VH6 | EVQLVESGGGLVQPGRSLRLSCAASGFTF |
| | | NDHWMTWVRQAPGKGLEWVSGITNTGG |
| | | ATYYADSVKGRFTISRDNAKNSLYLQMN |
| | | SLRAEDTALYYCTSPGLYFDYWGQGTM |
| | | VTVSS |
| 102 | Rat SG16.45 VL | EIVLTQSPTTTAASPGEKVTITCLATSSVS |
| | | VMYWYQHKSGASPKLLIYSTSSLASGVP |
| | | DRFSGSGSGTSYSLTINTMEAEDAATYYC |
| | | HQWSSDPPTFGSGTKLEIKR |
| 103 | hSG16.45 VK1 | EIVLTQSPGTLSASPGERVTISCRASSSVS |
| | | VMYWYQHKPGQAPRLLIYSTSSLASGVP |
| | | DRFSGSGSGTDYTLTISRMEPEDFAVYYC |
| | | HQWSSDPPTFGQGTKLEIKR |
| 104 | hSG16.45 VK2 | EIVLTQSPGTLSLSPGERATLSCRASSSVS |
| | | VMYWYQQKPGQAPRLLIYSTSSLASGIPD |
| | | RFSGSGSGTDFTLTISRLEPEDFAVYYCH |
| | | QWSSDPPTFGQGTKLEIKR |
| 105 | hSG16.45 VK3 | EIVLTQSPGTLSLSPGERATISCRASSSVSV |
| | | MYWYQHKPGQAPRLLIYSTSSLASGVPD |
| | | RFSGSGSGTDYTLTISRLEPEDFAVYYCH |
| | | QWSSDPPTFGQGTKLEIKR |
| 106 | hSG16.45 VK5 | EIVLTQSPGTLSLSPGERATLSCLATSSVS |
| | | VMYWYQHKPGQAPRLLIYSTSSLASGIPD |
| | | RFSGSGSGTDYTLTISRLEPEDFAVYYCH |
| | | QWSSDPPTFGQGTKLEIKR |

The anti-BCMA antibodies described herein may be a humanized or chimeric antibody of the rat SG16.17 or rat SG16.45 antibody.

In some embodiments, the anti-BCMA antibody described herein may comprise an hSG16.17 VH (e.g., any of VH1-VH6) paired with a hSG16.17 light chain (e.g., VL or any one of VK2-VK5). Any of such a combination is an hSG16.17 antibody. In other embodiments, the anti-BCMA antibodies may comprise an hSG16.45 heavy chain (e.g., any one of VH1-VH6) paired with an hSG16.45 light chain (e.g., any one of VK1-VK5). Any of such a combination is an hSG16.45 antibody. In some examples, the anti-BCMA antibody comprises the same mature heavy chain variable region as hSG16.45 VH5 and the same mature light chain variable region as hSG16.45 VK2. In another example, the anti-BCMA antibody comprises the same mature heavy chain variable region as hSG16.45 VH1 and the same mature light chain variable region as hSG16.45 VK1. In yet another example, the anti-BCMA antibody comprises the same mature heavy chain variable region as hSG16.45 VH1 and the same mature light chain variable region as hSG16.45 VK3.

In some embodiments, the anti-BCMA antibodies described herein bind to the same epitope in BCMA as any of the exemplary antibodies described herein, for example, hSG16.17 or hSG16.45, or competes against an exemplary antibody from binding to BCMA.

In some embodiments, the anti-BCMA antibody described herein contains a heavy chain CDR1 of DYY(I/M)H (SEQ ID NO: 107) or DHWMT (SEQ ID NO: 108), a heavy chain CDR2 of (Y/R/I)INPNSGYT(K/N/S)Y(N/A)(Q/E)(K/N)F(Q/K)(T/G) (SEQ ID NO: 109) or (S/A/G)ITNTGGATYY(L/A)DSVKG (SEQ ID NO: 110), and/or a heavy chain CDR3 of YMWERVTGFFDF (SEQ ID NO: 111) or PGLYFDY (SEQ ID NO: 112). Alternatively or in addition, the anti-BCMA antibody described herein contains a light chain CDR1 of (L/R)ASEDISDDLA (SEQ ID NO: 113) or (R/L)A(S/T)SSVSVMY (SEQ ID NO: 114), a light chain CDR2 of TTS(S/R)LQ(D/S) (SEQ ID NO: 115) or STSSLAS (SEQ ID NO: 116), and/or a light chain CDR3 of QQTYKFPPT (SEQ ID NO: 117) or HQWSSDPPT (SEQ ID NO: 118).

In some embodiments, the anti-BCMA antibodies comprise the same heavy chain CDRs as any of the exemplary heavy chains listed in Table 5 above and/or the same light chain CDRs as any of the exemplary light chains listed in Table 5.

Alternatively or in addition, the antibody may comprise a mature heavy chain variable region having at least 90% sequence identity (e.g., at least 90%, 91%, 92%, 93%, 94%, 95%, 96%, 97%, 98% or 99% sequence identity) to hSG16.17 VH3 and a mature light chain variable region having at least 90% sequence identity (e.g., at least 90%, 91%, 92%, 93%, 94%, 95%, 96%, 97%, 98% or 99% sequence identity) to hSG16.1.7 VK2.

Alternatively or in addition, the antibody may comprise a heavy chain mature variable region having at least 90% sequence identity (e.g., at least 90%, 91%, 92%, 93%, 94%, 95%, 96%, 97%, 98% or 99% sequence identity) to hSG16.45 VH5 and a mature light chain variable region having at least 90% sequence identity (e.g., at least 90%, 91%, 92%, 93%, 94%, 95%, 96%, 97%, 98% or 99% sequence identity) to hSG16.45 VK2.

In any of the anti-BCMA antibodies described herein, position H58 can be occupied by N or K, position H60 can be occupied by A or N, position H61 can be occupied by Q or E, position H62 can be occupied by K or N, position H64 can be occupied by Q or K, position H65 can be occupied by G or T, position L24 can be occupied by R or L and position L53 can be occupied by S or R. In some examples, positions H58, H60, H61, H62, H64 and H65 can be occupied by N, A, Q, K, Q and G respectively and/or L24 and L53 can be occupied by R and S, respectively. In other examples, positions H20, H48, H69, H71, H73, H76, H80, H88, H91 and H93 can be occupied by L, I, M, A, K, N, V, A, F, and T respectively, and/or positions L46, L48 and L87 can be occupied by V, V and F respectively. The numbering system used herein is the Kabat numbering system.

In any of the anti-BCMA antibodies described herein, positions H50 can be occupied by A or S, position L24 can be occupied by R or L, and/or position L26 can be occupied by S or T. Alternatively or in addition, positions H30, H93 and H94 can be occupied by N, T and S respectively.

In any of the antibodies described herein, the mature heavy chain variable region can be fused to a heavy chain constant region and the mature light chain variable region can be fused to a light chain constant region. Optionally, the heavy chain constant region is a mutant form of natural human constant region which has reduced binding to an Fcγ receptor relative to the natural human constant region. Optionally, the heavy chain constant region is of IgG1 isotype. Optionally, the heavy chain constant region and the light chain constant region have the sequences disclosed in Table 5. Optionally, the heavy chain constant region has an amino acid sequence comprising a S239C mutation. Optionally, the antibody is a naked antibody. Optionally, the antibody is conjugated to a cytotoxic or cytostatic agent. Optionally, the antibody is conjugated to a cytotoxic agent. Optionally, the cytotoxic agent is conjugated to the via an enzyme cleavable linker. Optionally, the cytotoxic agent is a DNA minor groove binder.

Insofar as humanized antibodies show any variation from the exemplified hSG16.17 VH3 VK2 humanized antibody, one possibility for such additional variation is additional backmutations in the variable region frameworks. Any or all of the positions backmutated in other exemplified humanized heavy or light chain mature variable regions can also be made (i.e., 1, 2, 3, 4, 5 or all 6) of H8 occupied by R, H67 occupied by A and H78 occupied by A, L40 occupied by S, L78 occupied by M and L85 occupied by D, or all 5 of H38 occupied by N, H40 occupied by R, H73 occupied by K, H82A occupied by S, and H83 occupied by T in the heavy chain and 1 or both of L3 occupied by K, and L20 occupied by I in the light chain.

Insofar as humanized antibodies show any variation from the exemplified hSG16.45 VHS VK2 humanized antibody, one possibility for such additional variation is additional backmutations in the variable region frameworks. Any or all of the positions backmutated in other exemplified humanized heavy or light chain mature variable regions can also be made (i.e., 1, 2, 3, or 4) of H37, H48, H76, H107 occupied by I, I, N, and V respectively and/or 1, 2, 3, 4, 5, 6 or 7 of L14, L19, L21, L38, L58, L71 and L78 occupied by A, V, I, H, V, Y, and M, respectively.

In some embodiments, the antibody may contain an Fc variant fragment which has an elevated binding affinity to a wild-type Fc receptor, for example, the Fc region of a wild-type IgG molecule (e.g., the Fc of rituximab or trastuzumab).

The Fc variant fragment may contain one or more mutation relative to the Fc of the wild-type IgG. In some embodiments, the one or more mutations in the Fc are located in the hinge and/or CH2 domain. Examples of mutations in the antibody known in the art and can be found, for example, in U.S. Pat. Nos. 7,601,335, 8,188,231, and 9,120,856, and include substitution mutations of amino acid residues S239, F243, R292, S298, Y300, V305, A330, 1332, E333, K334, or P396 (using EU index numbering as described in Kabat et al., (1991), Sequences of Proteins of Immunological Interest, 5th Ed.). In some embodiments, the one or more mutations in the Fc fragment can be S239D, F243L, R292P, S298A, Y300L, V305I, A330L, I332E, I332D, E333A, K334A, and/or P396L. See, for example, Shields et al. J. Biol. Chem. (2001) 276(9):6591-6604; Lazar et al. Proc. Natl. Acad. Sci. USA (2006) 103(11): 4005-4010; Stavenhagen et al. Cancer Res. (2007) 67(18): 8882-8890; Isoda et al. PLoS One (2015) 10(10): e0140120; Lu et al. J. Immunol. Met. (2011) 365:132-141; Liu et al. J. Biol. Chem. (2014) 289(6): 3571-3590; and Smith et al. Proc. Natl. Acad. Sci. USA (2012) 109(16):6181-6186. See also U.S. Pat. Nos. 6,737,056, 7,662,925, 7,317,091, and 8,217,147. The relevant disclosures of the referenced publications are incorporated by reference for the purposes or subject matter referenced herein.

In some embodiments, the Fc region may be glycoengineered (i.e., have altered glycosylation). In certain embodiments, the Fc region may be afucosylated (i.e., non-fucosylated).

Antibodies may be glycosylated at locations including conserved positions in their constant regions, affecting the function and structure of the antibody. See, e.g., Boyd et al., (1996) Mal. Immunol. 32:1311-1318; Wittwe and Howard, (1990) Biochem. 29:4175-4180; and Hefferis and Lund, supra; Wyss and Wagner, (1996) Current Opin. Biotech. 7:409-416). Altering the glycosylation (i.e., “glycoengineering”) through, for example, adding, shifting, or deleting one or more carbohydrate moieties found on an antibody or through changing the composition of the glycosylation (changing the glycosylation pattern). Altering the glycosylation of an antibody may be accomplished through the addition or substitution of at least one amino acid (e.g., 1, 2, 3, 4, 5, 6, or more amino acids) in the sequence of the antibody. Alternatively, the glycosylation of an antibody may also be accomplished without altering the sequence of the antibody. For example, the antibody may be expressed by cells that alter the glycosylation pattern of the antibody including, for example, cells that are genetically engineered to alter glycosylation patterns. See, e.g., Hse et al., (1997) J. Biol. Chem. 272:9062-9070 and Yang et al. (2015) Nature Biotechnology 33, 842-844. As a non-limiting example, the engineered cell line may have a loss-of-function mutation in the FUT8 gene. Other factors affecting glycosylation patterns during expression by cells may include media formulation, culture density, oxygenation, pH, purification schemes, and the like. See, e.g., U.S. Pat. Nos. 5,047,335, 5,510,261, and 5,278,299. Antibodies may also be processed after expression using, for example, enzymatic processes such as the use of endoglycosidases such as endoglycosidase H.

In certain embodiments, the antibodies for use with the methods described herein may have glycosylation patters with a lower amount of fucose when compared to wild-type antibodies. For example, in various embodiments, less than about 60%, less than about 50%, less than about 40%, less than about 30%, less than about 20%, less than about 15%, less than about 10%, less than about 5%, or less than about 3% of the molecules of an antibody have wild-type fucose levels. In some embodiments, about 2% of the molecules of the antibody have wild-type fucose levels. In one embodiment, the antibody comprises a fucose analog instead of fucose. Methods of making non-fucosylated antibodies by incubating antibody-producing cells with a fucose analogue, using fucose analogs, and fucose analogs per se are described, e.g., in WO2009135181 and WO2009135181. The relevant disclosures therein are incorporated by reference for the purpose and subject matter referenced.

(ii) Preparation of Anti-BCMA Antibodies

Antibodies capable of binding BCMA as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., BCMA or the catalytic domain thereof) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-BCMA monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of interfering with the BCMA activity. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of BCMA. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the BCMA polypeptide have been replaced (swapped) with sequences from a closely related, but antigenically distinct protein (such as another member of the neurotrophin protein family). By assessing binding of the antibody to the mutant BCMA, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-BCMA antibody is prepared by recombinant technology as exemplified below.

Nucleic acids encoding the heavy and light chain of an anti-BCMA antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-BCMA antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-BCMA antibody and the other encoding the light chain of the anti-BCMA antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-BCMA antibody as described herein, vectors (e.g., expression vectors) containing such; and host cells comprising the vectors are within the scope of the present disclosure.

(iii) Pharmaceutical Compositions of Anti-BCMA Antibodies

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPO™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### IV. Combined Immunotherapy of Immune Cells Expressing an ACTR Construct and Anti-BCMA Antibodies

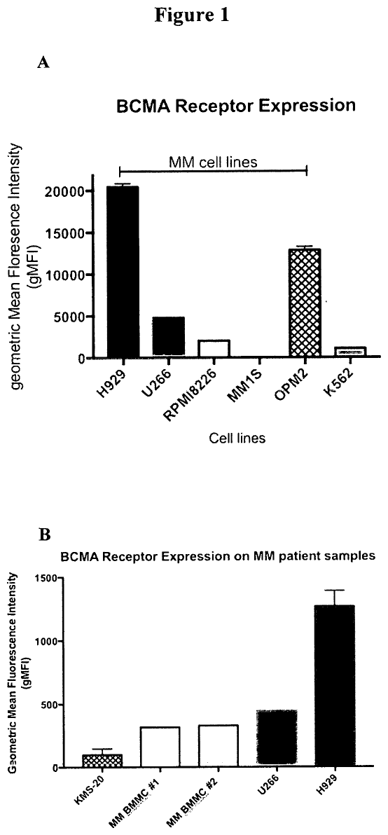

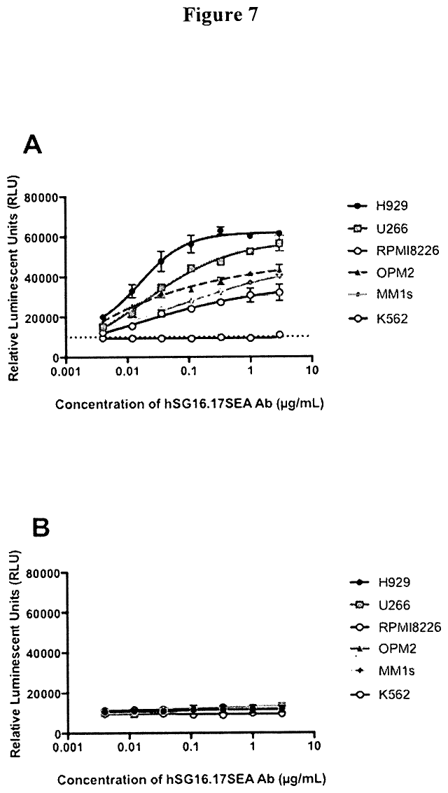

The exemplary ACTR constructs of the present disclosure confer antibody-dependent cell cytotoxicity (ADCC) capacity to T lymphocytes and enhance ADCC in NK cells. When the receptor is engaged by an anti-BCMA antibody bound to BCMA-expressing cells, it triggers T-cell activation, sustained proliferation and specific cytotoxicity against cells expressing BCMA. For example, this therapy would be effective against BCMA-positive cancer cells, B cells, or plasma cells. As a non-limiting set of examples, BCMA-positive cancer cells include hematological cancer cells (including, for example, myeloma, multiple myeloma, leukemia, lymphoma, or plasmacytoma cells). The effect of this therapy is specific to cells expressing BCMA as shown at least in FIG. 1 (comparing BCMA-positive (NCI-H929, U266B1, RPMI8226, MM1S, OPM2) with BCMA-negative (K562) cells); and FIG. 7 (comparing effects in BCMA-positive and negative cells with an anti-BCMA antibody both with and without an ACTR construct of the disclosure).

The degree of affinity of CD16 for the Fc portion of Ig is a critical determinant of ADCC and thus to clinical responses to antibody immunotherapy. The CD16 with the V158 polymorphism which has a high binding affinity for Ig and mediates superior ADCC was selected as an example. Although the F158 receptor has lower potency than the V158 receptor in induction of T cell proliferation and ADCC, the F158 receptor may have advantages relative to the V158 receptor in some clinical contexts.

The methods and ACTR constructs of the present disclosure facilitate T-cell therapy by allowing one single receptor to be used for all BCMA-positive cancers when combined with an anti-BCMA antibody. Antibody-directed cytotoxicity could be stopped whenever required by simple withdrawal of antibody administration. Clinical safety can be further enhanced by using mRNA electroporation to express the ACTR constructs transiently, to limit any potential autoimmune reactivity.

Thus, in one embodiment, the disclosure provides a method for enhancing efficacy of an antibody-based immunotherapy of a cancer in a subject in need thereof, which subject is being treated with an anti-BCMA antibody which can bind to BCMA-expressing cells and has a humanized Fc portion which can bind to human CD16, said method comprising introducing into the subject a therapeutically effective amount an anti-BCMA antibody and a therapeutically effective amount of T lymphocytes or NK cells, which T lymphocytes or NK cells comprise an ACTR construct of the disclosure.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a first pharmaceutical composition comprising an anti-BCMA antibody, and a second pharmaceutical composition comprising a population of T lymphocytes or NK cells that express an antibody-coupled T-cell receptor (ACTR) construct), the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

### A. Enhancing Immune Therapy Efficacy

Host cells (e.g., immune cells) expressing ACTR constructs described herein are useful for enhancing ADCC in a subject and/or for enhancing the efficacy of an antibody-based immunotherapy. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

To practice the method described herein, an effective amount of the immune cells (NK cells and/or T lymphocytes) expressing any of the ACTR constructs described herein and an effective amount of an anti-BCMA antibody, or compositions thereof may be administered to a subject in need of the treatment via a suitable route, such as intravenous administration. As used herein, an effective amount refers to the amount of the respective agent (e.g., the NK cells and/or T lymphocytes expressing ACTR constructs, anti-BCMA antibodies, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient. In other embodiments, the subject in need of treatment is a human with a B cell mediated disorder or a plasma cell mediated disorder.

The methods of the disclosure may be used for treatment of any BCMA-positive cancer. Specific non-limiting examples of cancers, which can be treated by the methods of the disclosure include, for example, hematological cancers such as myeloma (e.g., multiple myeloma or plasmacytoma), smoldering multiple myeloma (SMM), and monoclonal gammopathy of undetermined significance (MGUS)), leukemia, and lymphoma (e.g., non-Hodgkin's lymphoma (NHL, which includes Waldenström's macroglobulinemia (WM) and Burkitt lymphoma) or Hodgkin's lymphoma). In some embodiments, the cancer may be multiple myeloma in relapse or refractory multiple myeloma. Other BCMA-positive cancers may include myelodysplastic syndrome (MDS) or myeloproliferative syndrome (MPS). The methods of the disclosure may be used for treatment of any B cell mediated disorder or plasma cell mediated disorder. “B cell” as used herein refers to a type of white blood cell of the lymphocyte subtype that expresses B cell receptors on their cell membrane. Exemplary B cells include plasma cells, memory B cells, follicular B cells, marginal zone B cells, B-1 cells, and regulatory B cells. B cell mediated disorders (e.g., plasma cell mediated disorders) include, for example: rheumatoid arthritis, systemic lupus E (SLE), Type I diabetes, asthma, atopic dermatitis, allergic rhinitis, thrombocytopenic purpura, multiple sclerosis, psoriasis, Sjögren's syndrome, Hashimoto's thyroiditis, Graves' disease, primary biliary cirrhosis, Granulomatosis with polyangiitis (GPA; previously known as Wegener's granulomatosis or WG), tuberculosis, and graft-vs-host disease (GVHD).

In some embodiments, the immune cells are administered to a subject in an amount effective in enhancing ADCC activity by least 20% and/or by at least 2-fold, e.g., enhancing ADCC by 50%, 80%, 100%, 2-fold, 5-fold, 10-fold, 20-fold, 50-fold, 100-fold, or more.

The immune cells are co-administered with an anti-BCMA antibody in order to target BCMA-expressing cells. Anti-BCMA antibody-based immunotherapy may be used to treat, alleviate, or reduce the symptoms of any disease or disorder for which the immunotherapy is considered useful in a subject.

The efficacy of an antibody-based immunotherapy may be assessed by any method known in the art and would be evident to a skilled medical professional. For example, the efficacy of the antibody-based immunotherapy may be assessed by survival of the subject or tumor or cancer burden in the subject or tissue or sample thereof. In some embodiments, the immune cells are administered to a subject in need of the treatment in an amount effective in enhancing the efficacy of an antibody-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing the ACTR construct and/or the anti-BCMA antibody.

In any of the methods described herein, the immune cells (e.g., NK and/or T cells) may be autologous to the subject, i.e., the immune cells may be obtained from the subject in need of the treatment, genetically engineered for expression of the ACTR constructs, and then administered to the same subject. In one specific embodiment, prior to re-introduction into the subject, the autologous immune cells (e.g., T lymphocytes or NK cells) are activated and/or expanded ex vivo. Administration of autologous cells to a subject may result in reduced rejection of the host cells as compared to administration of non-autologous cells.

Alternatively, the host cells are allogeneic cells, i.e., the cells are obtained from a first subject, genetically engineered for expression of the ACTR construct, and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes in which the expression of the endogenous T cell receptor has been inhibited or eliminated. In one specific embodiment, prior to introduction into the subject, the allogeneic immune cells (e.g., T lymphocytes or NK cells) are activated and/or expanded ex vivo. T lymphocytes or NK cells can be activated by any method known in the art, e.g., in the presence of anti-CD3/CD28, IL-2, and/or phytohemoagglutinin. In certain embodiments, the ex vivo activation and/or expansion may be performed in the presence of an immunomodulatory agent (e.g., lenalidomide).

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line. See, e.g., U.S. Pat. Nos. 7,435,596 and 8,026,097 for the description of useful methods for expanding NK cells. For example, NK cells used in the methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)], and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the above methods, introduction (or re-introduction) of T lymphocytes, NK cells, or T lymphocytes and NK cells to the subject is followed by administering to the subject a therapeutically effective amount of IL-2.

The immune cells (e.g., T lymphocytes and/or NK cells) expressing any of the ACTR constructs disclosed herein may be administered to a subject who has been treated or is being treated with an Fc-containing anti-BCMA antibody. For example, the immune cells may be administered to a human subject simultaneously with an anti-BCMA antibody. Alternatively, the immune cells may be administered to a human subject during the course of an anti-BCMA antibody-based immunotherapy. In some examples, the immune cells and an anti-BCMA antibody can be administered to a human subject at least 4 hours apart, e.g., at least 12 hours apart, at least 1 day apart, at least 3 days apart, at least one week apart, at least two weeks apart, or at least one month apart.

In some embodiments, the human subject is an adult (i.e., is >18 years old). The human subject may have histologically- or cytologically-confirmed relapsed or refractory multiple myeloma (MM) with measurable disease. In some instances, the human subject may have received at least 3 prior lines of therapy to include treatment with a proteasome inhibitor (e.g., bortezomib, carfilzomib, or ixazomib) and an immunomodulatory agent (e.g., lenalidomide or pomalidomide) unless double-refractory to both; and a hematopoietic stem cell transplant (HSCT), for those subjects considered HSCT-eligible. In certain embodiments, the quantitative serum IgG levels for subjects with IgG MM may not exceed the institutional upper limit of normal (ULN). In some examples, the human subject may have an ECOG (Eastern Cooperative Oncology Group) performance status of 0 (i.e., the human subject is fully active, able to carry on all pre-disease performance without restriction) or 1 (i.e., the human subject is restricted in physically strenuous activity but ambulatory and able to carry out work of a light or sedentary nature, e.g., light house work or office work). Alternatively or in addition, the human subject may have a life expectancy of at least 6 months. In certain embodiments, the human subject may have an absolute neutrophil (ANC) count greater than 1000/4, a platelet count greater than 50,000/μL, an estimated glomerular filtration rate (GFR) or greater than 30 mL/min/1.73 m², or a combination thereof.

In some embodiments, the human subject may not (i) have a known active central nervous system (CNS) involvement (e.g., by multiple myeloma); (ii) have systemic rheumatic or autoimmune diseases or acute or chronic infections; (iii) have uncontrolled thromboembolic events or recent severe hemorrhage; (iv) currently use more than 5 mg/day of prednisone (or an equivalent glucocorticoid exceeding physiologic replacement levels); (v) have been administered any of the following prior treatments: T cell-directed antibody therapy (e.g., Alemtuzumab or anti-thymocyte globulin) within 6 months; any prior myeloma-directed therapy including cytotoxic chemotherapy, biologic therapy, or radiotherapy within 2 weeks; any monoclonal antibody or other protein therapeutic containing Fc-domains within 4 weeks; experimental agents within 3 half-lives, unless progression is documented on therapy; prior BCMA-directed investigational agents at any time; prior cell or gene therapy, excluding transfers of genetically unmodified autologous cells (e.g., hematopoietic stem cell transplantation), at any time; and/or prior allogeneic HSCT at any time; (vi) be pregnant or breastfeeding; or a combination of any one of (i)-(vi).

In accordance with the present disclosure, patients can be treated by infusing therapeutically effective doses of immune cells such as T lymphocytes or NK cells comprising an ACTR construct of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg. IL-2 can be co-administered to expand infused cells. The amount of IL-2 can about 1-5×10⁶ international units per square meter of body surface.

In some embodiments, the anti-BCMA antibody is administered to the subject in one or more doses of about 100-500 mg, 500-1000 mg, 1000-1500 mg or 1500-2000 mg. In some embodiments, the anti-BCMA antibody is administered to the subject in one or more doses of about 500 mg, about 600 mg, about 700 mg, about 800 mg, or about 900 mg. In some embodiments, the anti-BCMA antibody is administered to the subject in one or more doses of about 1000 mg, about 1100 mg, about 1200 mg, about 1300 mg, about 1400 mg, about 1500 mg, about 1600 mg, about 1700 mg, or about 1800 mg. In some embodiments, the anti-BCMA antibody is administered to the subject in one or more doses of about 1600 mg.

The particular dosage regimen, i.e., dose, timing and repetition, used in the method described herein will depend on the particular subject and that subject's medical history. The appropriate dosage of the anti-BCMA antibody used will depend on the type of cancer to be treated, the severity and course of the disease, previous therapy, the patient's clinical history and response to the antibody, and the discretion of the attending physician. The antibody can be administered to the patient at one time or over a series of treatments. The progress of the therapy of the disclosure can be easily monitored by conventional techniques and assays.

In some embodiments, the progress of the therapy of the disclosure may be measured by overall response rate (including, e.g., the International Myeloma Working Group (IMEG) response criteria for multiple myeloma), duration of response, period of progression-free survival, or overall survival. In certain embodiments, the persistence of the therapy may be measured by flow cytometry and/or by quantitative polymerase chain reaction (qPCR). In certain embodiments, inflammatory markers, cytokines, and/or chemokines may be measured after administration of the therapy of the disclosure. In certain embodiments, the plasma concentration of the anti-BCMA antibody may be ascertained.

The administration of the anti-BCMA antibody can be performed by any suitable route, including systemic administration as well as administration directly to the site of the disease (e.g., to a tumor).

In some embodiments, the method involves administering an anti-BCMA antibody (e.g., any anti-BCMA antibody disclosed herein) to the subject in one dose. In some embodiments, the method involves administering an anti-BCMA antibody to the subject in multiple dose (e.g., at least 2, 3, 4, 5, 6, 7, or 8 doses). In some embodiments, the anti-BCMA antibody is administered to the subject in multiple doses, with the first dose of the anti-BCMA antibody administered to the subject about 1, 2, 3, 4, 5, 6, or 7 days prior to administration of the immune cells expressing ACTR. In some embodiments, the first dose of the anti-BCMA antibody is administered to the subject between about 24-48 hours prior to the administration of the immune cells expressing ACTR.

In some embodiments, the anti-BCMA antibody is administered to the subject prior to administration of the immune cells expressing the ACTR and then subsequently about every two weeks. In some embodiments, the first two doses of the anti-BCMA antibody are administered about one week (e.g., about 6, 7, 8, or 9 days) apart. In certain embodiments, the third and following doses are administered about every two weeks.

In any of the embodiments described herein, the timing of the administration of the anti-BCMA antibody is approximate and includes three days prior to and three days following the indicated day (e.g., administration every three weeks encompasses administration on day 18, day 19, day 20, day 21, day 22, day 23, or day 24).

The efficacy of the methods described herein may be assessed by any method known in the art and would be evident to a skilled medical professional. For example, the efficacy of the antibody-based immunotherapy may be assessed by survival of the subject or cancer burden in the subject or tissue or sample thereof. In some embodiments, the antibody based immunotherapy is assessed based on the safety or toxicity of the therapy (e.g., administration of the anti-BCMA antibody and the immune cells expressing ACTR constructs) in the subject, for example by the overall health of the subject and/or the presence of adverse events or severe adverse events.

### B. Combination Treatments

The compositions and methods described in the present disclosure may be utilized in conjunction with other types of therapy for cancer, such as chemotherapy, surgery, radiation, gene therapy, and so forth. Such therapies can be administered simultaneously or sequentially (in any order) with the immunotherapy according to the present disclosure.

When co-administered with an additional therapeutic agent, suitable therapeutically effective dosages for each agent may be lowered due to the additive action or synergy.

The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The treatments of the disclosure may also be combined with one or more additional agents including, but not limited to: thalidomide (Thalomid®), derivatives or analogs of thalidomide such as lenalidomide (Revlimid®) or pomalidomide (Pomalyst®/Imnovid®), bortezomib (Velcade®), HDAC inhibitors such as panobinostat (Farydak®), ixazomib (Ninlaro®), daratumamab (Darzalex®), elotuzumab (Empliciti™), doxorubicin HCl (Doxil®), Carfilzomib (Kyprolis®), or steroids or corticosteroids such as dexamethasone and prednisone.

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers, and caspase activators; and chromatin disruptors.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### V. Kits for Therapeutic Use

The present disclosure also provides kits for use of the anti-BCMA antibody and a population of T lymphocytes or NK cells that express an antibody-coupled T-cell receptor (ACTR) construct in enhancing antibody-dependent cell-mediated cytotoxicity and enhancing an antibody-based immunotherapy. Such kits may include one or more containers comprising a first pharmaceutical composition that comprises an anti-BCMA antibody and a pharmaceutically acceptable carrier, and a second pharmaceutical composition that comprises a population of T lymphocytes and/or NK cells that express an antibody-coupled T-cell receptor (ACTR) construct such as those described herein. The population of T lymphocytes and/or NK cells may further express an exogenous polypeptide comprising a co-stimulatory domain or a ligand of a co-stimulatory factor.

In some embodiments, the kit can additionally comprise instructions for use in any of the methods described herein. The included instructions may comprise a description of administration of the first and second pharmaceutical compositions to a subject to achieve the intended activity, e.g., enhancing ADCC activity, and/or enhancing the efficacy of an antibody-based immunotherapy, in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the first pharmaceutical composition is an anti-BCMA antibody as described herein. At least one active agent in the second pharmaceutical composition is a population of T lymphocytes or NK cells that express an antibody-coupled T-cell receptor (ACTR) construct as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and H (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: BCMA Expression on the Surface of Multiple Myeloma Cell Lines and Patient Samples

The expression of BCMA on the surface of five different multiple myeloma cell lines was determined by flow cytometry. In FIG. 1, part A, multiple myeloma (MM) cell lines were washed twice in Phosphate Buffered Saline (PBS) with BSA (staining buffer) before incubation with 10 μg/mL of SG16.17 for 30 min at 4 degrees Celsius. This was followed by two washes in staining buffer and incubation of PE-conjugated goat anti-human IgG (Fab′)₂ secondary antibody for 30 min at 4 degrees C. Following two washes in staining buffer, PE detection was performed via flow cytometry. The geometric mean fluorescence intensity (gMFI) of the PE signal for each cell line is graphed. In FIG. 1, part B, the surface expression staining of BCMA was re-run on KMS-20, NCI-H929 and U266B1 cells along with MM patient-derived bone marrow mononuclear cells (BMMC) via flow cytometry. In this experiment, the geometric mean fluorescence intensity (gMFI) of BCMA within the MM plasma cell population was compared between three multiple myeloma cell lines and two patient BMMC samples. Patient derived BMMC (MM#1 and MM#2) were thawed and washed twice in staining buffer before being stained with a Live/Dead eFluor-780 dye for 30 min at 4 degrees C. After the live/dead cell staining, cells were washed twice and incubated with 100 μL of an antibody cocktail consisting of AF488-conjugated anti-human CD38 antibody, anti-human CD138 V450, anti-human CD45 Brilliant Violet 605, anti-human IgG Brilliant Violet 711, anti-CD19-PE, anti-CD56 PerCP-Cy5.5, anti-CD27-BV510, and AF647 conjugated SG16.17 anti-BCMA antibody. After staining, cells were washed twice again with staining buffer and staining detected via flow cytometry. Flow cytometry gating on multiple myeloma cell lines and MM patient derived BMMC was performed after doublet exclusion and dead cell exclusion.

### Example 2: Binding of ACTR T-Cells to SG16.17 and Afucosylated (SEA) SG16.17 Antibodies

Gamma-retrovirus was generated that encoded a construct expressing ACTR variant SEQ ID NO: 1. This virus was used to infect primary human T-cells, generating cells that expressed this ACTR variant on the surface of infected cells.

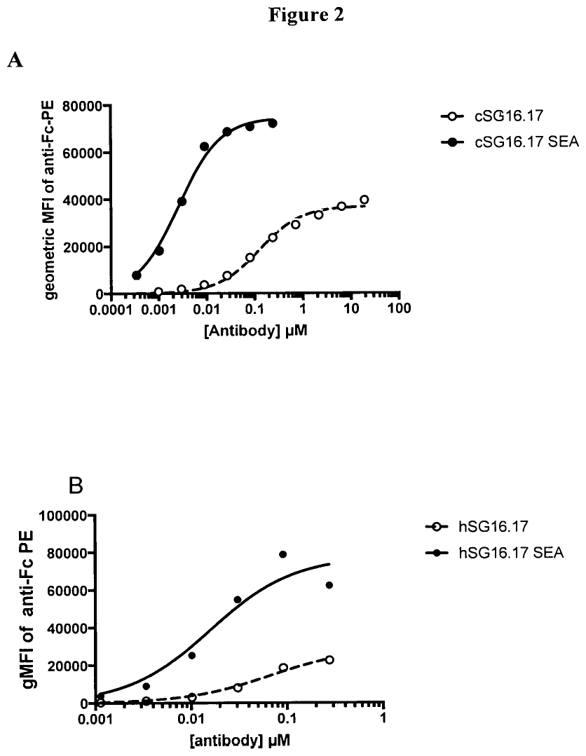

The ability of antibody to bind to these ACTR T cells was assessed via flow cytometry. ACTR-transduced T cells were incubated with varying concentrations of chimeric (FIG. 2, part A) and humanized (FIG. 2, part B) versions of the SG16.17 and SG16.17SEA (afucosylated) antibodies in X VIVO-15 media for 30 minutes at 37 degrees C. After the incubation, cells were placed on ice and washed twice with cell staining buffer before being stained with an antibody cocktail that consisted of PE-conjugated goat anti-human IgG (Fab′)₂ antibody and anti-human CD16-APC for 30 minutes at 4 degrees C. Following two washes in staining buffer, detection was performed via flow cytometry. The geometric mean fluorescence intensity (gMFI) of the PE signal within the CD16+ ACTR T cells is presented in FIG. 2. These results demonstrate that the afucosylated (SEA) version of the SG16.17 antibody had increased binding or apparent affinity when compared to the fucosylated antibody for both chimeric and humanized versions.

### Example 3: Cytotoxicity of Multiple Myeloma Cell Lines in the Presence of Various BCMA Antibodies Coupled to ACTR T-Cells

ACTR T-cells were generated as described in Example 2. Antibody staining for CD16 expression followed by flow cytometry demonstrated that 46-56% of the T cells expressed ACTR. These cells were used in cytotoxicity assays with NCI-H929 and U266B1 target cells that constitutively expressed firefly luciferase.

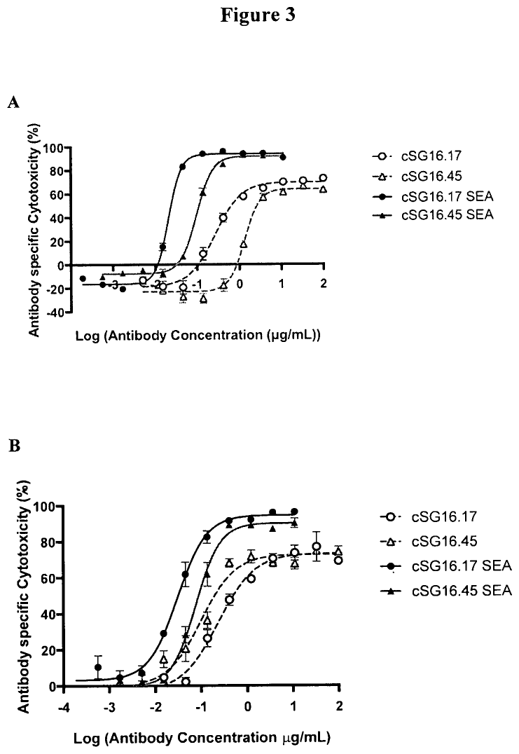

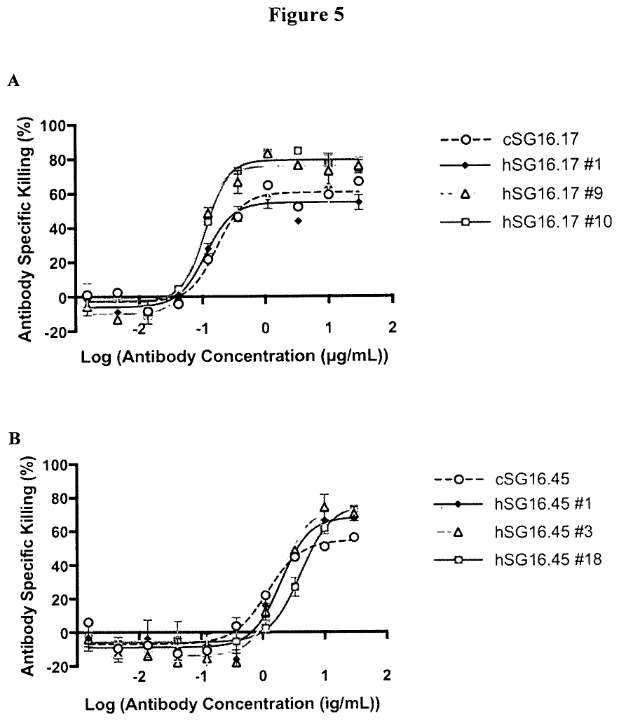

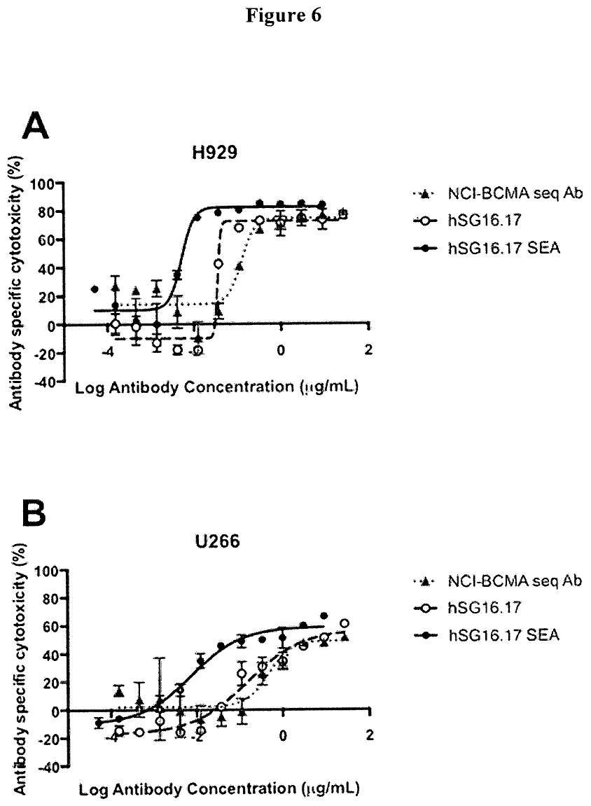

T-cells expressing ACTR variant SEQ ID NO: 1 (effector; E) and target cells (target; T) were incubated at a 4:1, effector-to-target ratio (120,000 T cells; 30,000 target cells) with increasing concentrations of various chimeric anti-BCMA antibodies (FIG. 3, parts A and B), humanized anti-BCMA antibody clones (FIG. 5, parts A and B), or anti-BCMA antibodies derived from the NCI-murine BCMA CAR sequence shown in WO2010104949A2 (FIG. 6, parts A and B) in a 200-μL reaction volume with RPMI 1640 media supplemented with 10% fetal bovine serum. The chimeric and humanized antibodies analyzed included fucosylated and afucosylated (labeled as “SEA”) versions of the SG16.17 and SG16.45 anti-BCMA antibodies. Reactions were incubated in a CO₂ (5%) incubator at 37 degrees C. for 24 or 48 hours. An equal volume of Bright-Glo luciferase assay reagent (Promega; Madison, Wis.) was added prior to a 10 minute room temperature incubation. Luminescence was measured using an EnVision Multi-label plate reader (PerkinElmer; Waltham, Mass.). The percentage of live cells was determined by dividing the luminescence signal of a given sample by the luminescence signal in the absence of antibody and multiplying by 100. The percent cytotoxicity was determined by subtracting the percentage of live cells from 100. The percentage of antibody specific cytotoxicity (%) was then plotted as a function of antibody concentration.

In FIG. 3, the results demonstrated a dose-dependent increase in (A) NCI-H929 and (B) U266B1 target cell cytotoxicity following co-culture with ACTR T-cells in the presence of increasing concentrations of one of the four chimeric anti-BCMA antibodies after 24 and 48 hours of co-culture. Further, the afucosylated (SEA) versions of the chimeric antibodies (cSG16.17SEA and cSG16.45SEA) demonstrated increased cytotoxicity when compared with the fucosylated antibodies. In FIG. 5, part A, results demonstrated that all three of the presented humanized clones of SG16.17 performed similarly to, if not better (clones #9 and #10) than, the chimeric SG16.17 antibody. In FIG. 5, part B, the results demonstrated that all three humanized clones of SG16.45 performed similarly to the chimeric SG16.45 antibody with NCI-H929 cells. In FIG. 6, the results demonstrated that the humanized afucosylated BCMA antibody, SG16.17SEA, demonstrated increased cytotoxicity when compared to the anti-BCMA antibody derived from NCI-murine BCMA sequence shown in WO2010104949 A2 for both NCI-H929 (FIG. 6, part A) and U266B1 (FIG. 6, part B) multiple myeloma cell lines. Similar results were obtained with RPMI-8226 multiple myeloma cells.

### Example 4: ACTR T-Cell Cytokine Release in the Presence of Various BCMA Antibodies Opsonized to BCMA Expressing NCI-H929 and U266B1 Target Cells

ACTR T-cells were generated as described in Example 2. Antibody staining for CD16 expression followed by flow cytometry demonstrated that 46-49% of the T cells expressed ACTR. These cells were used in co-culture assays with NCI-H929 and U266B1 target cells to collect supernatants for cytokine analysis.

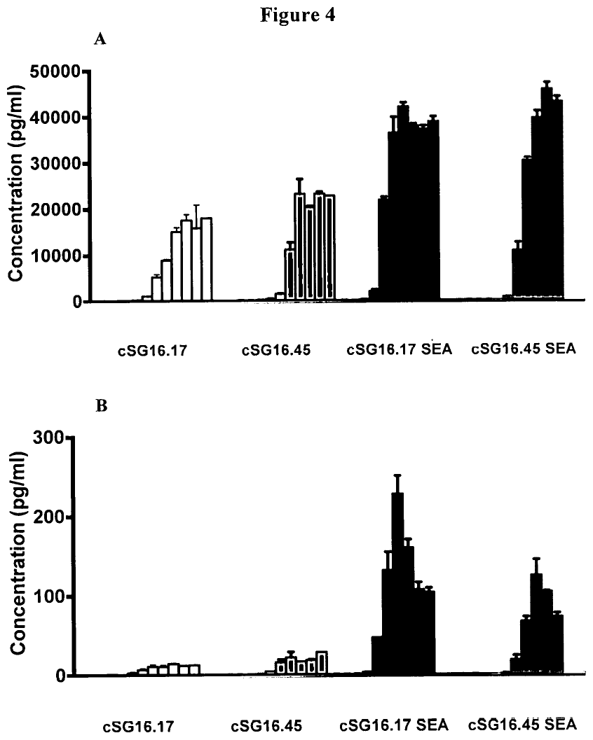

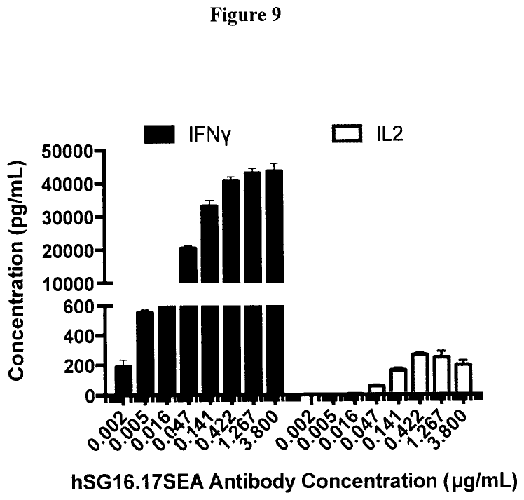

T-cells expressing ACTR variant SEQ ID NO: 1 (effector; E) and target cells (target; T) were incubated at a 4:1, effector-to-target ratio (120,000 T cells; 30,000 target cells) with increasing concentrations of various chimeric antibodies in a 200-μL reaction volume in RPMI 1640 media supplemented with 10% fetal bovine serum. In FIG. 4, the chimeric antibodies included fucosylated and afucosylated (SEA) versions of the SG16.17 and SG16.45 anti-BCMA antibodies at a concentration range of 100-0.0005 μg/mL. In FIG. 9, humanized afucosylated (SEA) SG16.17 antibody was used at a concentration range of 3.8-0.002 μg/mL. After 24 hours, half of the reaction volume (100 μL) was collected and frozen at −20 degrees C. Supernatants were subsequently analyzed for IFNγ and IL2 using the Meso Scale Discovery V-Plex Human IFN-γ Kit (K151QOD-4) and V-Plex Human IL-2 Kit (K151QQD-4). Briefly, the Proinflammatory Calibrator Blend, Detection Antibody Solution, and Read Buffer were prepared according to manufacturer protocols. Co-culture supernatants were thawed on ice and diluted appropriately in RPMI 1640 media supplemented with 10% fetal bovine serum. 50 μL of the diluted sample or calibrator was added to the MSD plate, and the plate was sealed and incubated on a shaker at 600×g for 2 hours in the dark. The plate was then washed three times with 150 μL PBS containing 0.05% Tween-20. 25 μL of detection antibody solution was added to the plate, and the plate was sealed and incubated on a shaker at 600×g for 2 hours in the dark. The plate was washed three times with 150 μL Phosphate Buffered Saline containing 0.05% Tween-20. Read Buffer (150 μL) was added to the plate and the plates were run on the MSD Quickplex SQ 120.

All chimeric and humanized antibodies opsonized on NCI-H929 cells induced increasing ACTR T-cell IFNγ (FIG. 4, part A; FIG. 9) and IL2 (FIG. 4, part B; FIG. 9) release with increasing antibody concentration. When NCI-H929 target cells were incubated with Mock T-cells, minimal cytotoxicity, IFNγ and IL2 release were observed (data not shown). These results demonstrate that ACTR T-cells were able to secrete cytokine in an anti-BCMA antibody concentration-dependent manner with opsonized target cells. The afucosylated (SEA) versions of the antibodies (SG16.17SEA and SG16.45SEA) demonstrated increased potency in cytokine release when compared to their fucosylated counterparts.

### Example 5: Jurkat Reporter Cell Line Activation in the Presence of BCMA-Expressing Myeloma Cell Lines and Increasing Concentration of hSG16.17SEA

The ability of anti-BCMA antibody opsonized target cells to activate Jurkat cells was analyzed in a Jurkat cell reporter assay reflective of T cell activation. Jurkat cells were transduced with lentivirus encoding firefly luciferase downstream of a minimal CMV promoter element and tandem repeats of the nuclear factor of activated T-cells (NFAT) consensus binding site. In this cell line, upregulation of NFAT transcription factors, which is a marker of cell activation, resulted in binding to the transcriptional response elements and subsequent expression of luciferase, which was monitored by measuring luminescence following luciferase cleavage of the luciferin substrate. As such, luciferase expression is reflective of cell activation. This cell line is designated as Jurkat-N.

Jurkat-N cells were mixed at a 1:1 ratio with BCMA positive target MM cells such as NCI-H929, U266B1, RPMI-8226, OPM-2 and MM1s cells in a 100-μL reaction volume in RPMI-1640 media supplemented with 10% fetal bovine serum. K562, a surface BCMA-negative leukemia cell line, was used as a control. Reactions were incubated in the presence of a 3-0.004 μg/mL of humanized hSG16.17SEA antibody for 5 hours in a CO₂ (5%) incubator at 37 degrees C. After the 5 hour incubation, britelite plus reagent (100 μL, Perkin Elmer) was added to lyse the cells and add the luciferin substrate. Reactions were then incubated for 10 minutes in the dark and luminescence was measured using an EnVision Multi-label plate reader (Perkin-Elmer; Waltham, Mass.).

Results from Jurkat-N cells transduced with retrovirus expressing ACTR variant SEQ ID NO: 1 (Jurkat-N-ACTR) are shown in FIG. 7, part A. Results from Jurkat-N cells lacking ACTR expression were used as a negative control and are shown in FIG. 7, part B.

Robust activation of Jurkat-N ACTR cells was observed in the presence of hSG16.17SEA antibody and BCMA-expressing multiple myeloma cell lines as evidenced by an increased luminescence signal; an increase in luminescence was not observed with K562 cells, a leukemia cell line that lacks surface BCMA expression (FIG. 7, part A). Further, Jurkat-N cells lacking ACTR did not demonstrate an increase in luminescence in the presence of hSG16.17SEA antibody and target cells (FIG. 7, part B). These results demonstrated that only target cells expressing surface BCMA facilitated the activation of Jurkat-N cells expressing ACTR in the presence of a BCMA targeting antibody. Furthermore, in the absence of a targeting antibody (basal signal; dotted line) activation of the Jurkat-N-ACTR cells was not observed (FIG. 7, part A).

### Example 6: Cytotoxicity of NCI-H929 and U266B1 Multiple Myeloma Cell Lines in the Presence of Humanized Afucosylated (SEA) BCMA Antibody Coupled to ACTR T-Cells

ACTR T-cells were generated as described in Example 2. Antibody staining for CD16 expression followed by flow cytometry demonstrated that 46% of T cells expressed ACTR. Mock T-cells (T-cells not expressing ACTR variants) were expanded in a similar fashion to the ACTR T cells. Both the Mock T cells and ACTR T cells were used in cytotoxicity assays with NCI-H929 and U266B1 target cells that constitutively expressed firefly luciferase.

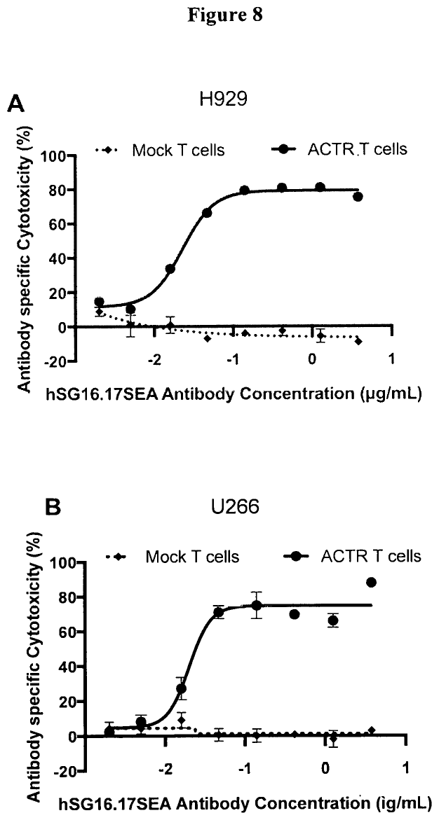

In FIG. 8, T-cells expressing ACTR variant SEQ ID NO: 1 (effector; E) and target cells (target; T) were incubated at a 4:1, effector-to-target ratio (120,000 T cells; 30,000 target cells) with increasing concentrations of humanized afucosylated (SEA) anti-BCMA antibody SG16.17 in a 200-μL reaction volume in RPMI 1640 media supplemented with 10% fetal bovine serum. Target-cell luminescence was measured and percent cytotoxicity was determined as in Example 3.

An increase in NCI-H929 and U266B1 target cell cytotoxicity was observed when the cells were co-cultured for 24 and 48 hours with T-cells expressing ACTR variant SEQ ID NO: 1 and increasing concentrations of hSG16.17SEA (FIG. 8). This increase in cytotoxicity was antibody dependent and ACTR T-cell specific, as basal cytotoxicity was low in the absence of antibody and with Mock T-cells.

### Example 7: ACTR T-Cell Proliferation in the Presence of Humanized Afucosylated (SEA) Anti-BCMA Antibody and BCMA Expressing Multiple Myeloma Cell Lines

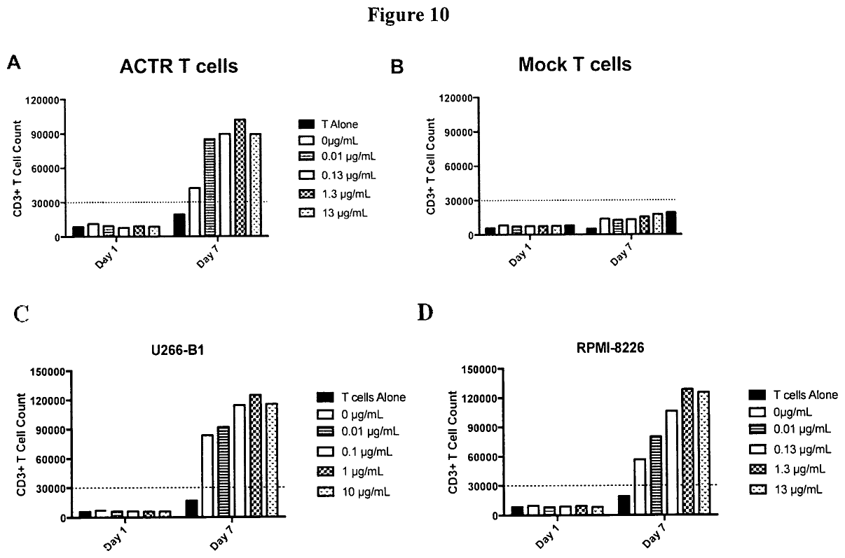

ACTR T-cells were generated as described in Example 2. Antibody staining for CD16 expression followed by flow cytometry demonstrated that 33% of T cells expressed ACTR. Mock T-cells (T-cells not expressing ACTR variants) were expanded in a similar fashion to the ACTR T cells. Both ACTR T-cells (FIG. 10, parts A, C, and D) and Mock T-cells (FIG. 10, part B) were used in the proliferation assays with BCMA expressing MM target cell lines NCI-H929 (FIG. 10, parts A and B); U266B1 (FIG. 10, part C), and RPMI-8226 (FIG. 10, part D).

T-cells expressing ACTR variant SEQ ID NO: 1 (effector; E) and target cells (target; T) were incubated at a 1:1, effector-to-target ratio (30,000 T cells; 30,000 target cells) with increasing concentrations of humanized afucosylated (SEA) BCMA antibody in a 200-μL reaction volume in RPMI 1640 media supplemented with 10% fetal bovine serum and 25 U/mL IL2. Reactions were incubated in a CO₂ (5%) incubator at 37 degrees C. for 7 days. The number of live CD3+ T-cells were determined at day 1 and day 7 and compared to the input number of T-cells (dotted line). Briefly, at day 1 and day 7, cells were washed twice in staining buffer before being stained with a Live/Dead eFluor-780 dye for 30 min at 4 degrees C. After incubating in the live/dead cell stain, cells were washed twice and subsequently incubated with 100 μL of an antibody cocktail consisting of AF488-conjugated anti-human CD3 antibody and AF647-conjugated anti-human CD16 antibody. After staining, cells were washed twice again with staining buffer and staining was subsequently detected via flow cytometry. Flow cytometry gating on CD3+ T lymphocytes was performed after doublet exclusion and dead cell exclusion.

In FIG. 10, results demonstrate an increase in proliferation of CD3+ T cells of ACTR variant SEQ ID NO: 1 after 7 days of co-culture with BCMA positive target cells in the presence of increasing concentration of humanized hSG16.17SEA. The increase in T-cell proliferation was antibody dependent and ACTR T-cell specific, as basal proliferation was low in the absence of antibody and with Mock T-cells. Some basal proliferation (absence of antibody) was observed with U266B1 cells possibly due to allogeneic stimulation. ACTR+ T-cell proliferation follows the same trend as the CD3+ T cells (data not shown).

### Example 8: ACTR T-Cell Expansion in the Presence of Humanized Afucosylated (SEA) Anti-BCMA Antibody and BCMA-Expressing Multiple Myeloma Cell Lines

ACTR T-cells were generated as described in Example 2. Antibody staining for CD16 expression followed by flow cytometry demonstrated that 63% of T cells expressed ACTR. Mock T-cells (T-cells not expressing ACTR variants) were expanded in a similar fashion to the ACTR T cells. Both ACTR T cells and Mock T cells were used in restimulation or T cell expansion assays with target cells such as BCMA expressing NCI-H929 cell line and BCMA negative K562 cells.

T-cells (effector; E) and target cells (target; T) were incubated at a 1:1, effector-to-target ratio (30,000 T cells; 30,000 target cells) with 0.1 mg/mL of humanized hSG16.17SEA in a 200-μL reaction volume in RPMI 1640 media supplemented with 10% fetal bovine serum and 25 U/ml IL2. Reactions were incubated in a CO₂ (5%) incubator at 37° C. Every 3-4 days, the live CD3+ T cell count was measured by flow cytometry by staining with a live/dead stain and an anti-CD3 antibody, in manner similar to that described in Example 7; cells were also stained with an anti-CD16 antibody to measure ACTR+ cells. T cells were restimulated at a 1:2 ratio with target cells every 3-4 days based on the CD3+ T cell count; 0.1 μg/mL of humanized hSG16.17SEA was also added at each restimulation.

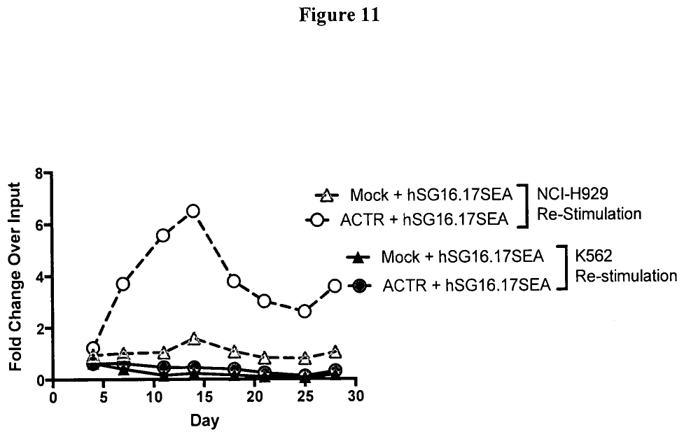

Results with T-cells expressing ACTR variant SEQ ID NO: 1 are shown in FIG. 11 and demonstrate 28-day expansion of CD3+ T-cells when they were re-stimulated with BCMA-positive NCI-H929 target cells, but not BCMA-negative K562 cells in the presence of 0.1 μg/mL humanized hSG16.17SEA. The increase in T-cell proliferation was ACTR T-cell specific, as it was not observed with Mock T-cells. ACTR+ T-cell proliferation follows the same trend as the CD3+ T-cells (data not shown).

### Example 9: ACTR T-Cells in Combination with hSG16.17SEA Antibody in a Subcutaneous NCI-H929 Model of Multiple Myeloma in NSG Mice

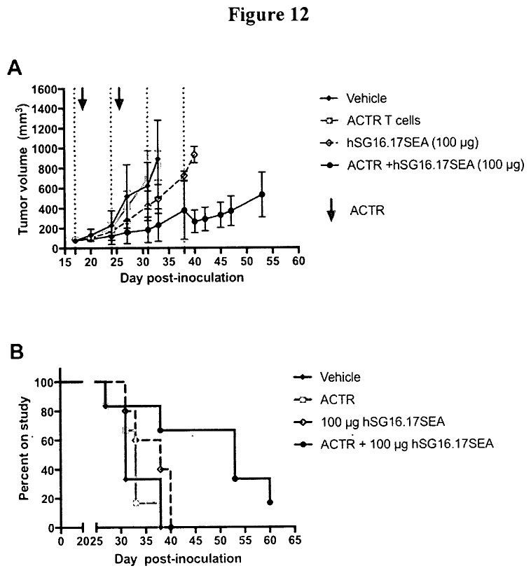

A subcutaneous NCI-H929 MM xenograft model was established by inoculation of a suspension of NCI-H929 cells in 1:1 Matrigel:serum-free media (1×10⁵ cells per mouse) into the flank of female NSG mice. When mean tumor volume reached 80-100 mm³, mice were randomized into treatment groups. Each treatment group had 5 or 10 mice, and the treatment groups were as follows: vehicle (PBS alone), hSG16.17SEA antibody alone, ACTR variant SEQ ID NO: 1 T-cells alone, and ACTR variant SEQ ID NO: 1 T-cells in combination with hSG16.17SEA antibody. Groups treated with hSG16.17SEA antibody were dosed with 100 μg of antibody per mouse once a week for 4 weeks (QWx4, represented by dotted vertical lines on the graph in FIG. 12); groups treated with ACTR T-cells were dosed with 1×10⁷ cells per mouse dosed once a week for 2 weeks (QWx2, represented on the graph in FIG. 12 by grey arrows). Mice were monitored daily, and body weights and tumor volume measurements were taken twice weekly for the duration of the study. Mice were euthanized when the tumor volume exceeded 1000 mm³. In FIG. 12, part A, the mean tumor volume was plotted as a function of time for mice in the different treatment arms. The data demonstrated that ACTR T-cells in combination with hSG16.17SEA induced greater tumor growth inhibition than either single agent alone. The percentage of mice remaining on study was plotted as a function of time to predefined tumor volume end-point of mice in the different treatment arms (FIG. 12, part B). Treatment with ACTR T-cells in combination with hSG16.17SEA antibody prolonged time to a pre-defined tumor volume end-point when compared to hSG16.17SEA antibody alone.

### Example 10: Cytotoxicity of NCI-H929 and U266B1 Multiple Myeloma Cell Lines with Increasing E:T Ratios of ACTR T Cells and Anti-BCMA Antibodies

ACTR T-cells were generated as described in Example 2. Antibody staining for CD16 expression followed by flow cytometry demonstrated that 49% of T cells expressed ACTR. These cells were used in cytotoxicity assays with NCI-H929 and U266B1 target cells that constitutively expressed firefly luciferase.

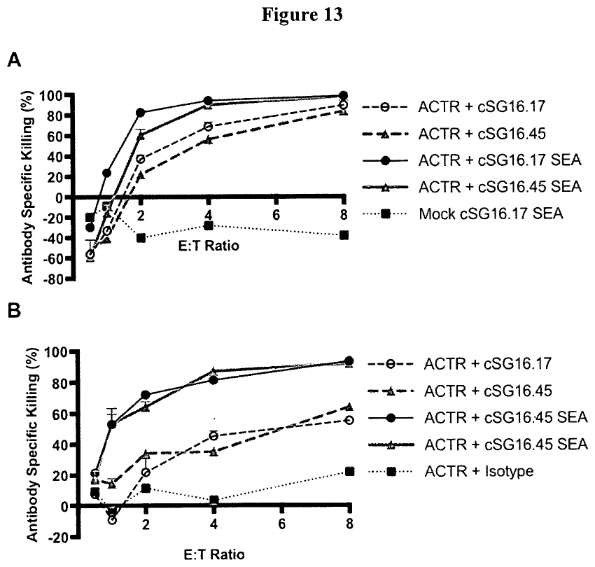

T-cells (effector; E) and target cells (target; T) were incubated at varying effector-to-target (E:T) cell ratios in the presence of 10 μg/mL anti-BCMA antibody (FIG. 13), in a 200-μL reaction volume in RPMI 1640 media supplemented with 10% fetal bovine serum. The antibodies included fucosylated and afucosylated (SEA) versions of the two anti-BCMA antibodies (SG16.17 and SG16.45). Reactions were incubated in a CO₂ (5%) incubator at 37 degrees C. for 24 hours for NCI-H929 cells (FIG. 13, part A) or 48 hours for U266B1 cells (FIG. 13, part B). Target cell luminescence was measured and percent cytotoxicity was determined as in Example 3.

The results with T-cells expressing ACTR variant SEQ ID NO: 1 are shown in FIG. 13 and demonstrated an increase in NCI-H929 and U266B1 target cell cytotoxicity with increasing numbers of ACTR T-cells in the presence of a fixed concentration of the four anti-BCMA antibodies. The afucosylated (SEA) versions of the chimeric antibodies (cSG16.17SEA and cSG16.45SEA) demonstrated increased potency in comparison to the fucosylated antibodies. Basal cytotoxicity in the presence of isotype antibody and with Mock T-cells was observed to be very low in both target cell lines.

### Example 11: Activation of ACTR T-Cells in the Presence of Anti-BCMA Antibody Opsonized NCI-H929 and U266B1 Multiple Myeloma Cell Lines

ACTR T-cells were generated as described in Example 2. Antibody staining for CD16 expression followed by flow cytometry demonstrated that 46% of T cells expressed ACTR.

T-cells (effector; E) and target cells (target; T) were incubated at a 1:1, effector-to-target ratio (30,000 T cells; 30,000 target cells) with 1 μg/mL of anti-BCMA antibodies in a 200-μL reaction volume in RPMI 1640 media supplemented with 10% fetal bovine serum. Reactions were incubated in a CO₂ (5%) incubator at 37 degrees C. for 24 hours. Geometric Mean Fluorescence intensity (gMFI) of two activation markers, CD25 and CD69 were determined in the total CD3+ CD16+ ACTR T-cell gate via flow cytometry. Briefly, cells were washed twice in staining buffer before being stained with a Live/Dead eFluor-450 dye for 30 min at 4 degrees C. After incubating in the live/dead cell stain, cells were washed twice and incubated with 100 μL of an antibody cocktail consisting of AF488-conjugated anti-human CD3 antibody, APC-conjugated anti-human CD16 antibody, PerCP-Cy5.5-conjugated anti-human CD25 antibody, and BV510-conjugated anti-human CD69 antibody. Subsequently, cells were washed twice, fixed, permeabilized, and stained for intracellular molecules prior to detection via flow cytometry. Flow cytometry gating on CD3+ CD16+ ACTR T-cells was performed after doublet and dead cell exclusion.

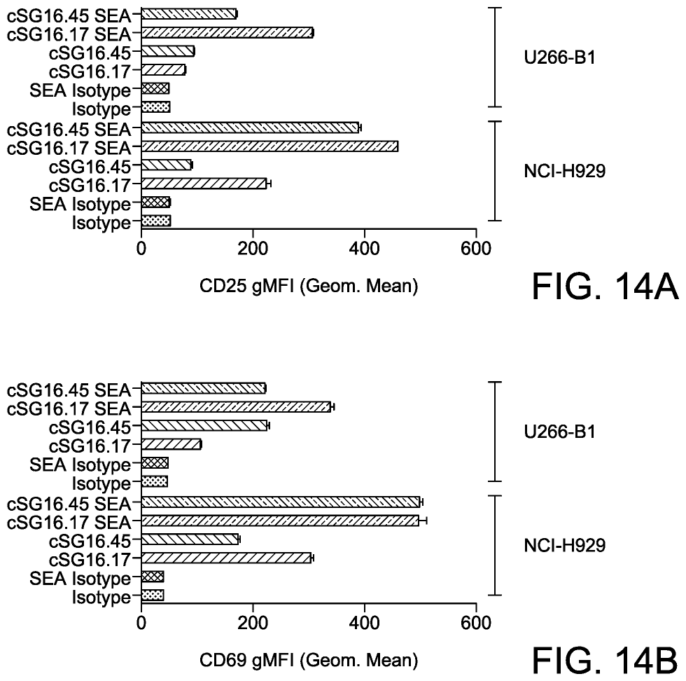

The results with T-cells expressing ACTR variant SEQ ID NO: 1 are shown in FIG. 14 and demonstrated a significant increase in levels of activation markers CD25 (FIG. 14, part A) and CD69 (FIG. 14, part B) on ACTR T-cells in 24 hours when co-cultured with BCMA positive target cells, NCI-H929 and U266B1, in the presence of anti-BCMA antibodies. ACTR T cells incubated with afucosylated (SEA) versions of the antibodies (cSG16.17SEA and cSG16.45SEA) demonstrated enhanced expression of CD25 and CD69 when compared to incubations with their fucosylated counterparts. The isotype-control antibodies induced minimal to no activation of ACTR T-cells in the presence of NCI-H929 and U266B1 target cells.

### Example 12: Specificity of hSG16.17 SEA Coupled ACTR T-Cell Cytokine Production in the Presence of BCMA-Positive and BCMA-Negative Target Cells

ACTR or mock T-cells (effector; E) and target cells (target; T) were incubated at a 4:1 E:T ratio in the presence of increasing concentrations of anti-BCMA (hSG16.17 SEA) or anti-HER2 (trastuzumab) targeting antibodies. For these experiments, BCMA+ HER2− NCI− H929 and BCMA− HER2+ SKBR3 target cells were used. Cell supernatants were collected following a 24-hour incubation in a 37 degrees C./5% CO₂ incubator. Supernatants were subsequently analyzed for IFNγ using the Meso Scale Discovery V-Plex Human IFN-γ Kit (K151QOD-4). Briefly, the Proinflammatory Calibrator Blend, Detection Antibody Solution, and Read Buffer were prepared according to manufacturer protocols. Co-culture supernatants were thawed on ice and diluted appropriately in RPMI 1640 media supplemented with 10% fetal bovine serum. 50 μL of the diluted sample or calibrator was added to the MSD plate, and the plate was sealed and incubated on a shaker at 600×g for 2 hours in the dark. The plate was then washed three times with 150 μL PBS containing 0.05% Tween-20. 25 μL of detection antibody solution was added to the plate, and the plate was sealed and incubated on a shaker at 600×g for 2 hours in the dark. The plate was washed three times with 150 μL Phosphate Buffered Saline containing 0.05% Tween-20. Read Buffer (150 μL) was added to the plate and the plates were run on the MSD Quickplex SQ 120.

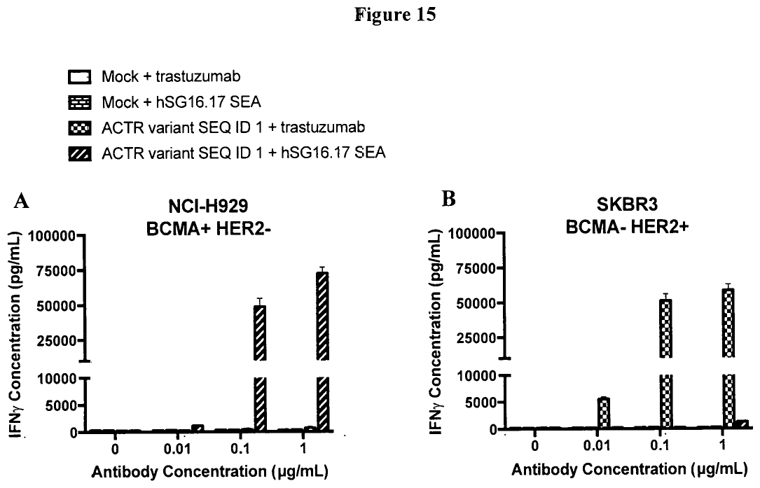

When T-cells expressing ACTR SEQ ID NO:1 were incubated with increasing concentrations of hSG16.17 SEA antibody and BCMA+ HER2− NCI-H929 target cells, a concentration-dependent increase in IFNγ production was observed (FIG. 15, panel A); similar results were observed when T cells expressing ACTR SEQ ID NO:1 were incubated with increasing concentrations of HER2-targeting trastuzumab antibody and BCMA− HER2+ SKBR3 target cells (FIG. 15, panel B). ACTR T-cells produced minimal IFNγ in the presence of hSG16.17 SEA antibody and BCMA− HER2+ SKBR3 cells (FIG. 15, panel B), demonstrating that the observed cytokine production was target specific. ACTR T-cells also produced minimal IFNγ in the presence of trastuzumab and BCMA-positive NCI-H929 cells (FIG. 15, panel A), demonstrating that the observed effector function was specific to the anti-BCMA hSG16.17 SEA antibody. These experiments demonstrate that IFNγ production by ACTR-expressing T cells is dependent on a matched antibody-target pair.

### Example 13: Cytotoxicity of NCI-H929 Target Cells in the Presence of Increasing Effector to Target Cell Ratios

Gamma-retroviruses were generated that encoded ACTR variant SEQ ID NO: 57 or SEQ ID NO: 58. These viruses were used to infect primary human T-cells, generating cells that expressed these ACTR variants on the surface of infected cells. These cells were subsequently used in cytotoxicity assays with BCMA-positive NCI-H929 target cells and BCMA-targeting hSG16.17 SEA antibody. Mock T-cells (T-cells not expressing ACTR variants) were expanded in a similar fashion to the ACTR T cells and used as controls in this experiment.

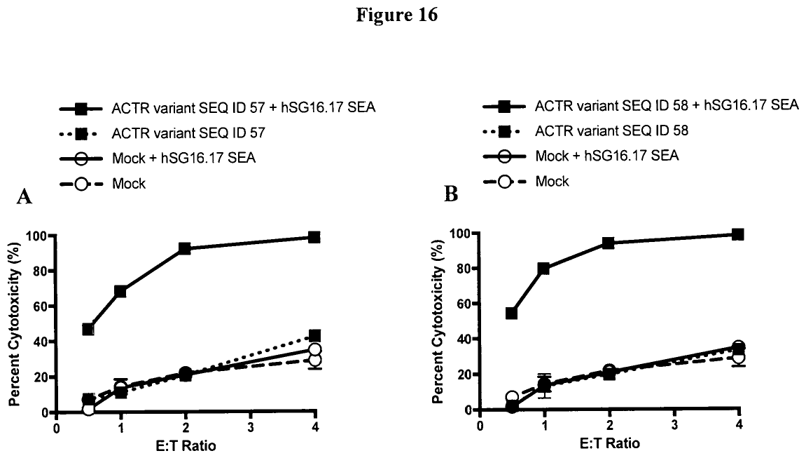

T-cells (effector; E) and NCI-H929 target cells (target; T) were incubated at varying effector-to-target ratios in the presence of 1 μg/mL hSG16.17 SEA antibody. Reactions were incubated in a CO₂ (5%) incubator at 37 degrees C. for 44 hours followed by subsequent flow cytometry staining. Briefly, cells were washed twice with PBS followed by staining with a fixable viability dye. Cells were washed again with PBS, followed by incubation with 100 μL of antibody cocktail containing AlexaFluor488-conjugated anti-human CD3, AlexaFluor 647-conjugated anti-human CD16, and PerCP-Cy5.5-conjugated anti-human CD25 antibodies. Following a thirty-minute incubation, cells were washed twice and data acquired on a flow cytometer. Live target cell counts were determined by gating on viability dye negative, CD3− CD16− cells. The percentage of live target cells was determined by dividing the live target cell count from a given sample by the live target cell count in the target cell alone wells. The percent cytotoxicity was determined by subtracting the percent live cells from 100. Percent cytotoxicity was plotted as a function of E:T ratio (FIG. 16).

A T-cell dose dependent increase in cytotoxicity was observed for ACTR T-cells expressing ACTR variant SEQ ID NO: 57 (FIG. 16, panel A) and ACTR variant SEQ ID NO: 58 (FIG. 16, panel B) when cultured in the presence of NCI-H929 target cells and hSG16.17 SEA antibody. ACTR T-cells in the absence of antibody and mock T-cells in the absence and presence of antibody demonstrated minimal cytotoxicity (FIG. 16, panels A and B).

### Example 14: Upregulation of CD25 Activation Marker on ACTR T Cells in the Presence of Increasing Effector to Target Cell Ratios

Gamma-retroviruses were generated that encoded ACTR variant SEQ ID NO: 57 or SEQ ID NO: 58. These viruses were used to infect primary human T-cells, generating cells that expressed these ACTR variants on the surface of infected cells. These cells were subsequently used in activation assays with BCMA-positive NCI-H929 target cells and BCMA-targeting hSG16.17 SEA antibody. Mock T-cells (T-cells not expressing ACTR variants) were expanded in a similar fashion to the ACTR T cells and used as controls in this experiment.

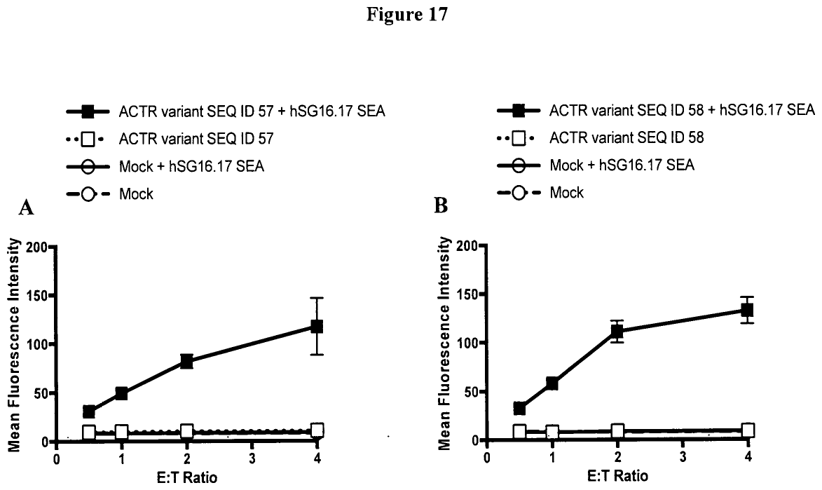

T-cells (effector; E) and NCI-H929 target cells (target; T) were incubated at varying effector-to-target ratios in the presence of 1 μg/mL hSG16.17 SEA antibody. Reactions were incubated in a CO₂ (5%) incubator at 37 degrees C. for 44 hours followed by subsequent flow cytometry staining. Briefly, cells were washed twice with PBS followed by staining with a fixable viability dye. Cells were washed again with PBS, followed by incubation with 100 μL of antibody cocktail containing AlexaFluor488-conjugated anti-human CD3, AlexaFluor 647-conjugated anti-human CD16, and PerCP-Cy5.5-conjugated anti-human CD25 antibodies. Following a thirty-minute incubation, cells were washed twice and data acquired on a flow cytometer. The geometric mean fluorescence intensity of CD25 was calculated within the CD3+ T-cell gate and was plotted as a function of E:T ratio (FIG. 17).

T-cells expressing ACTR variant SEQ ID NO: 57 (FIG. 17, panel A) and ACTR variant SEQ ID NO: 58 (FIG. 17, panel B) showed an increase in CD25 expression relative to ACTR T cells in the absence of antibody. Similarly, low levels of CD25 were observed on ACTR T cells in the absence of antibody and on mock T cells in the absence and presence of antibody.

### Example 15: hSG16.17 SEA Coupled ACTR T-Cell Cytotoxicity is Concentration-Dependent in the Presence of BCMA-Positive Target Cells and not BCMA-Negative Target Cells

Gamma-retroviruses were generated that encoded ACTR variant SEQ ID NO: 57 or SEQ ID NO: 58. These viruses were used to infect primary human T-cells, generating cells that expressed these ACTR variants on the surface of infected cells. Mock T-cells (T-cells not expressing ACTR variants) were expanded in a similar fashion to the ACTR T cells. Both the Mock T-cells and ACTR variant T-cells were used in cytotoxicity assays with NCI-H929 and K562 target cells that constitutively expressed firefly luciferase.

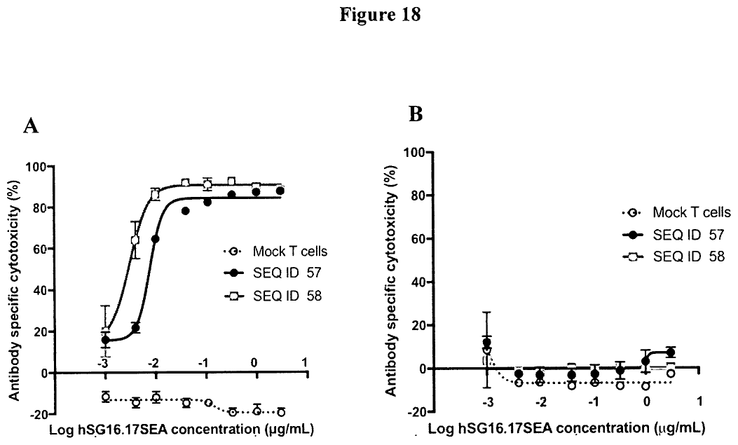

In FIG. 18, T-cells (effector; E) and target cells (target; T) were incubated at a 4:1, effector-to-target ratio (30,000 target cells) with increasing concentration of humanized afucosylated anti-BCMA antibody hSG16.17SEA in a 200-μL reaction volume in RPMI 1640 media supplemented with 10% fetal bovine serum. Reactions were incubated at 37 degrees C. in a 5% CO₂ incubator for 48 hr. Target cell luminescence was measured and percent cytotoxicity was determined as described in Example 3.

An increase in target cell cytotoxicity was observed with T cells expressing ACTR variants SEQ ID NO: 57 and SEQ ID NO: 58 in the presence of BCMA-expressing NCI-H929 cells and increasing concentrations of hSG16.17SEA (FIG. 18, panel A). No increase in cytotoxicity was observed with T cells expressing ACTR variants SEQ ID NO: 57 and SEQ ID NO: 58 in the presence of BCMA-negative K562 cells and increasing concentrations of hSG16.17SEA (FIG. 18, panel B). This NCI-H929 cytotoxicity was antibody dependent and ACTR T-cell specific, as minimal cytotoxicity was observed in the presence of Mock T-cells. Similar results were observed with BCMA-expressing U266B1 cells (data not shown).

### Example 16: ACTR T-Cell Cytokine Release in the Presence of hSG16.17SEA Opsonized to BCMA Expressing NCI-H929 Target Cells

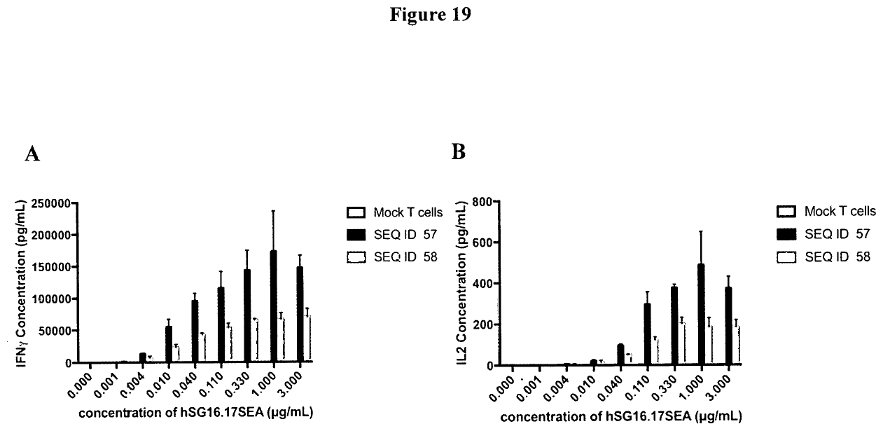

Gamma-retroviruses were generated that encoded ACTR variant SEQ ID NO: 57 or SEQ ID NO: 58. These viruses were used to infect primary human T-cells, generating cells that expressed these ACTR variants on the surface of infected cells. Mock T-cells (T-cells not expressing ACTR variants) were expanded in a similar fashion to the ACTR T cells. These effector cells were used in co-culture assays with BCMA-positive, NCI-H929 and U266B1 target cells to collect supernatants for cytokine analysis in FIG. 19.

Mock or ACTR T-cells (effector; E) and target cells (target; T) were incubated at a 4:1, effector-to-target ratio (120,000 T cells; 30,000 target cells) with increasing concentrations of hSG16.17SEA antibody (0-3 μg/mL) in a 200-μL reaction volume in RPMI 1640 media supplemented with 10% fetal bovine serum. Reactions were incubated at 37 degrees C. in a 5% CO₂ incubator for 48 hr. Half of the reaction volume (100 μL) was collected and frozen at −20 degrees C. Supernatants were subsequently analyzed for IFNγ and IL2 using the Meso Scale Discovery V-Plex Human IFN-γ Kit (K151QOD-4) and V-Plex Human IL-2 Kit (K151QQD-4). Briefly, the Proinflammatory Calibrator Blend, Detection Antibody Solution, and Read Buffer were prepared according to manufacturer protocols. Co-culture supernatants were thawed on ice and diluted appropriately in RPMI 1640 media supplemented with 10% fetal bovine serum. 50 μL of the diluted sample or calibrator was added to the MSD plate, and the plate was sealed and incubated on a shaker at 600×g for 2 hours in the dark. The plate was then washed three times with 150 μL PBS containing 0.05% Tween-20. 25 μL of detection antibody solution was added to the plate, and the plate was sealed and incubated on a shaker at 600×g for 2 hours in the dark. The plate was washed three times with 150 μL Phosphate Buffered Saline containing 0.05% Tween-20. Read Buffer (150 μL) was added to the plate and the plates were run on the MSD Quickplex SQ 120.

T cells expressing ACTR variants SEQ ID NO: 57 and SEQ ID NO: 58 showed antibody-dependent and antibody-concentration-dependent IFNγ (FIG. 19, panel A) and IL2 (FIG. 19, panel B) release in the presence of hSG16.17SEA and NCI-H929. Low or no cytokine release was observed with mock T cells. These results demonstrated that ACTR T-cells were able to secrete cytokine in an ACTR-specific, antibody-dependent and hSG16.17SEA concentration-dependent manner with BCMA-positive target cells. Similar results were observed in the presence of BCMA-expressing U266B1 cells (data not shown). In similar experiments, IFNγ and IL-2 release was also observed with additional ACTR variants with both NCI-H929 and U266B1 BCMA-positive target cell lines. The IFNγ and IL-2 release observed with 1 μg/mL hSG16.17SEA antibody from these experiments can be found in Table 6 and 7. These experiments demonstrate that multiple ACTR variants showed hSG16.17SEA-dependent cytokine release in the presence of BCMA-positive target cell lines.

TABLE 6
IL2 Cytokine Release from T cells expressing ACTR variants

| | IL-2 Release (pg/mL) | |
|---|---|---|
| | NCI-H929 Target Cells + | U266B1 Target Cells + |
| SEQ ID NO | hSG16.17SEA | hSG16.17SEA |
| 7 | 262.13 | 34.84 |
| 59 | 46.59 | 213.92 |
| 57 | 372.52 | 186.79 |
| 60 | 258.48 | 48.57 |
| 61 | 178.50 | 132.08 |
| 62 | 119.11 | 12.31 |
| 63 | 66.54 | 1.60 |
| 10 | 224.01 | 260.21 |
| 58 | 419.70 | 17.83 |

TABLE 7
IFNγ cytokine release from T cells expressing ACTR variants

| | IFNγ Release (pg/mL) | |
|---|---|---|
| | NCI-H929 Target Cells + | U266B1 Target Cells + |
| SEQ ID NO | hSG16.17SEA | hSG16.17SEA |
| 7 | 6249.7 | 13673.2 |
| 59 | 17140.6 | 12626.6 |
| 57 | 16737.7 | 12560.5 |
| 60 | 18649.7 | 14356.9 |
| 61 | 22891.4 | 11573.4 |
| 62 | 14496.1 | 7578.0 |
| 63 | 16000.2 | 10358.5 |
| 10 | 19830.3 | 7969.5 |
| 58 | 16782.5 | 12666.6 |

### Example 17: hSG16.17 SEA-Coupled ACTR T Cell Proliferation in the Presence of BCMA Positive Multiple Myeloma Cell Lines

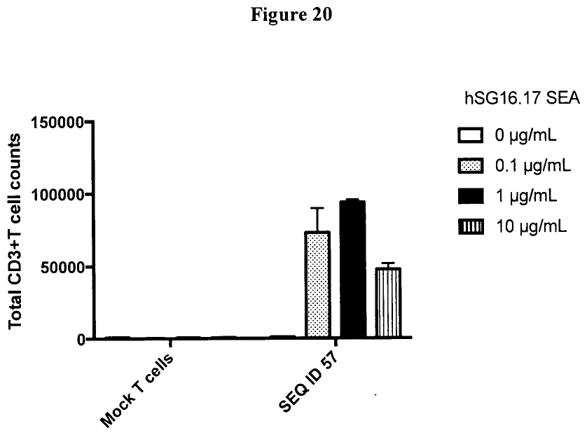

Gamma-retroviruses were generated that encoded ACTR variant SEQ ID NO: 57. These viruses were used to infect primary human T-cells, generating cells that expressed these ACTR variants on the surface of infected cells. Mock T-cells (T-cells not expressing ACTR variants) were expanded in a similar fashion to the ACTR T cells. Both ACTR T cells and Mock T cells were used in the proliferation assays with BCMA expressing MM target cell lines such as NCI-H929 in FIG. 20.

Mock T cells or ACTR T cells (effector; E) and target cells (target; T) were incubated at a 1:1, effector-to-target cell ratio (30,000 target cells) with increasing concentration of humanized afucosylated anti-BCMA antibody (hSG16.17SEA) in a 200-μL reaction volume in RPMI 1640 media supplemented with 10% fetal bovine serum. Reactions were incubated in a CO₂ (5%) incubator at 37 degrees C. for 7 days. The counts of live CD3+ T cells were determined at day 7. Briefly, at day 7, cells were washed twice in staining buffer before being stained with a Live/Dead eFluor-780 dye for 30 min at 4 degrees C. After incubating in the live/dead cell stain, cells were washed twice and subsequently incubated with 100 of an antibody cocktail consisting of AF488-conjugated anti-human CD3 antibody and AF647-conjugated anti-human CD16 antibody. After staining, cells were washed twice again with staining buffer and staining was subsequently detected via flow cytometry. Flow cytometry gating on CD3+ T lymphocytes was performed after doublet exclusion and dead cell exclusion.

In FIG. 20, results demonstrate an increase in proliferation of CD3+ T cells after a 7 day co-culture with ACTR T cell variant SEQ ID NO: 57, BCMA-positive NCI-H929 target cells and increasing concentrations of hSG16.17SEA. This increase in T cell proliferation was antibody dependent and ACTR T-cell specific, as proliferation was minimal in the absence of antibody and with Mock T-cells.

In additional experiments, T-cell proliferation was evaluated in the presence of 1 μg/mL hSG16.17SEA under conditions similar to those described above with T-cells expressing a number of different ACTR variants. The increase in the number of CD3+ cells relative to reactions without antibody was determined. These ACTR variants showed antibody-dependent proliferation (Table 8). These experiments demonstrate that multiple ACTR variants in combination with hSG16.17SEA showed antibody-dependent T cell proliferation in the presence of BCMA-positive target cell lines.

TABLE 8
Antibody-dependent proliferation of T cells
expressing different ACTR variants

Fold increase at 1 μg/mL relative to no antibody

| SEQ ID NO | NCI-H929 Target Cells | U266B1 Target Cells |
|---|---|---|
| 7 | 80.07 | 52.00 |
| 59 | 11.51 | 6.91 |
| 60 | 18.34 | 11.67 |
| 61 | 15.85 | 8.72 |
| 62 | 6.24 | 2.87 |
| 57 | 35.51 | 49.48 |
| 63 | 3.76 | 2.82 |
| 10 | 7.46 | 1.62 |
| 58 | 9.76 | 4.75 |

### Example 18: ACTR T Cell Proliferation in the Presence of BCMA+ Multiple Myeloma Target Cells, hSG16.17 SEA Antibody, and the Immunomodulatory Drug, Lenalidomide

Mock or ACTR T cells (effector; E) and target cells (target; T) were incubated at a 1:1, effector-to-target cell ratio in the presence and absence of the anti-BCMA antibody, hSG16.17 SEA (1 μg/mL). Proliferation assays were carried out with NCI-H929 and RPMI-8226 target cells in the absence or presence of lenalidomide (1 μM). The total CD3+ T cell count was determined by flow cytometry after a 7 day incubation in a CO₂ (5%) incubator. Briefly, cells were washed once in PBS before staining with a fixable viability dye. Following the incubation with viability dye, cells were washed with cell staining buffer and subsequently stained with 100 μL of an antibody cocktail consisting of AF488-conjugated anti-human CD3 antibody and AF647-conjugated anti-human CD16 antibody. After staining, cells were washed twice with staining buffer and detected via flow cytometry. Flow cytometry gating on CD3+ T lymphocytes was performed after dead cell exclusion. Total CD3+ T cell count was plotted as a function of lenalidomide concentration.

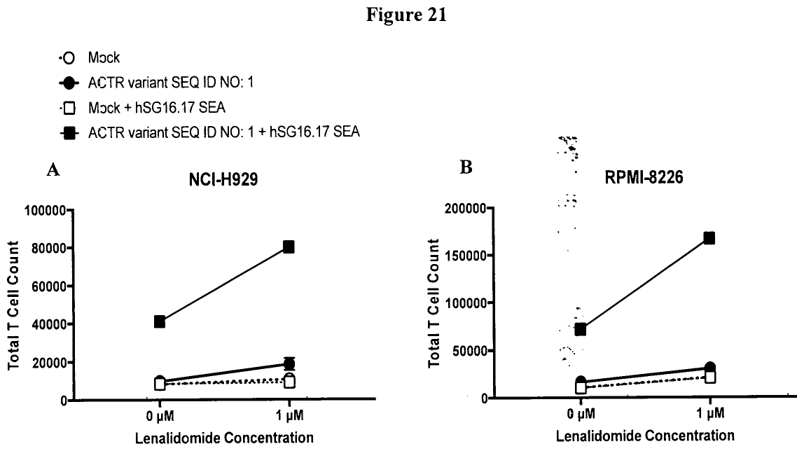

An increase in CD3+ T cell count was observed when T cells expressing ACTR variant SEQ ID NO: 1 were cultured in the presence of BCMA+ target cells and hSG16.17 SEA antibody. Lenalidomide further enhanced the proliferation of hSG16.17 SEA coupled ACTR T cells in the presence of NCI-H929 (FIG. 21, panel A) and RPMI-8226 (FIG. 21, panel B) multiple myeloma target cells. This increase in T cell proliferation was antibody dependent and ACTR T cell specific, as minimal proliferation was observed in the absence of antibody and with Mock T-cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

1. A method of enhancing antibody-dependent cell cytotoxicity (ADCC) in a subject, the method comprising administering to a subject in need thereof a therapeutically effective amount of an anti-BCMA antibody and an effective amount of immune cells, wherein the immune cells are T lymphocytes or NK cells expressing an antibody-coupled T-cell receptor (ACTR) construct, which comprises:
(a) an Fc binding domain;
(b) a transmembrane domain; and
(c) a cytoplasmic signaling domain comprising an immunoreceptor tyrosine-based activation motif (ITAM).

2. The method of claim 1, wherein the ACTR construct further comprises:
(d) at least one co-stimulatory signaling domain; and
wherein either (c) or (d) is located at the C-terminus of the ACTR construct; optionally wherein (c) is located at the C-terminus of the ACTR construct.

3. The method of claim 1, wherein the ACTR further comprises (e) a hinge domain, which is located at the C-terminus of (a) and the N-terminus of (b).

4. The method of claim 1, wherein the Fc binding domain of (a) is selected from the group consisting of:
(i) an extracellular ligand-binding domain of an Fc-receptor, which optionally is an Fc-gamma receptor, an Fc-alpha receptor, or an Fc-epsilon receptor,
(ii) an antibody fragment that binds the Fc portion of an immunoglobulin,
(iii) a naturally-occurring protein that binds the Fc portion of an immunoglobulin, or an Fc-binding fragment thereof, and
(iv) a synthetic polypeptide that binds the Fc portion of an immunoglobulin.

5. The method of claim 4, wherein the Fc binding domain is (i), which is an extracellular ligand-binding domain of a CD16A, CD32A, or CD64A receptor, wherein optionally the extracellular ligand-binding domain of CD16A is CD16A F158 or CD16A V158.

6. The method of claim 4, wherein the Fc binding domain is (ii), which is a single chain variable fragment (ScFv), a domain antibody, or a nanobody.

7. The method of claim 4, wherein the Fc binding domain is (iii), which is Protein A or Protein G.

8. The method of claim 4, wherein the Fc binding domain is (iv), which is a Kunitz peptide, a SMIP, an avimer, an affibody, a DARPin, or an anticalin.

9. The method of claim 1, wherein the transmembrane domain of (b) is of a single-pass membrane protein.

10. The method of claim 9, wherein the transmembrane domain is of a membrane protein selected from the group consisting of CD8α, CD8β, 4-1BB, CD28, CD34, CD4, FcεRIγ, CD16A, OX40, CD3ζ, CD3ε, CD3γ, CD3δ, TCRα, CD32, CD64, VEGFR2, FAS, PD1, and FGFR2B.

11. The method of claim 1, wherein the transmembrane domain of (b) is a non-naturally occurring hydrophobic protein segment.

12. The method of claim 2, wherein the at least one co-stimulatory signaling domain of (d) is of a co-stimulatory molecule selected from the group consisting of 4-1BB, CD28, CD28_{LL→GG} variant, OX40, ICOS, CD27, GITR, HVEM, TIM1, LFA1, and CD2.

13. The method of claim 2, wherein the ACTR construct comprises a single co-stimulatory signaling domain or two co-stimulatory signaling domains.

14. The method of claim 13, wherein the ACTR construct comprises two co-stimulatory domains, which are from:
(i) CD28 and 4-1BB;
(ii) CD28_{LL→GG} variant and 4-1BB;
(iii) CD28 and CD27;
(iv) CD28 and OX40.

15. The method of claim 1, wherein the cytoplasmic signaling domain of (c) is a cytoplasmic domain of CD3ζ or FcεR1γ.

16. The method of claim 3, wherein the hinge domain of (e) is of CD8α, CD28, or IgG.

17. The method of claim 3, wherein the hinge domain of (e) is a non-naturally occurring peptide.

18. The method of claim 17, wherein the hinge domain of (e) is an extended recombinant polypeptide (XTEN) or a (Gly₄Ser)ₙ polypeptide, in which n is an integer of 3-12, inclusive.

19. The method of claim 1, wherein the ACTR construct further comprises a signal peptide at its N-terminus.

20. The method of claim 19, wherein the signal peptide is of CD8α.

21. The method of claim 3, wherein the hinge domain is 1 to 60 amino acid residues in length, optionally wherein the hinge domain is 1 to 30 amino acid residues in length or 31 to 60 amino acid residues in length.

22. The method of claim 3, wherein the ACTR construct comprises (i) a single co-stimulatory domain of 4-1BB or CD28, and (ii) a hinge domain of CD8 or CD28, a transmembrane domain of CD8 or CD28, or a combination thereof.

23. The method of claim 22, wherein the ACTR construct comprises (i) a 4-1BB co-stimulatory signaling domain, and (ii) a CD8 hinge domain, a CD8 transmembrane domain, or a combination thereof.

24. The method of claim 22, wherein the ACTR construct comprises (i) a CD28 co-stimulatory signaling domain, and (ii) a CD28 hinge domain, a CD28 transmembrane domain, or a combination thereof.

25. The method of claim 1, wherein the ACTR construct is free of a hinge domain from any non-CD16A receptor, and optionally wherein the ACTR construct is free of any hinge domain.

26. The method of claim 25, wherein the ACTR construct comprises a CD28 co-stimulatory signaling domain and optionally a CD8 transmembrane domain.

27. The method of claim 1, wherein the ACTR construct comprises the amino acid sequence selected from the group consisting of SEQ ID NOs: 1-63.

28. The method of claim 1, wherein the subject is a human patient having a BCMA-positive cancer.

29. The method of claim 28, wherein the BCMA-positive cancer is a hematological cancer, which optionally is myeloma, leukemia, or lymphoma.

30. The method of claim 1, wherein the subject is a human patient having a B cell mediated disorder, which optionally is selected from the group consisting of rheumatoid arthritis, systemic lupus E (SLE), Type I diabetes, asthma, atopic dermatitis, allergic rhinitis, thrombocytopenic purpura, multiple sclerosis, psoriasis, Sjögren's syndrome, Hashimoto's thyroiditis, Graves' disease, primary biliary cirrhosis, Granulomatosis with polyangiitis (GPA), tuberculosis, and graft-vs-host disease (GVHD).

31. The method of claim 1, wherein the T lymphocytes or NK cells are autologous T lymphocytes or autologous NK cells isolated from the subject.

32. The method of claim 1, wherein the T lymphocytes or NK cells are allogeneic T lymphocytes or allogeneic NK cells.

33. The method of claim 31, wherein, prior to administering into the subject, the T lymphocytes or NK cells are activated and/or expanded ex vivo, and wherein optionally the ex vivo activation and/or expansion is performed in the presence of an immunomodulatory agent, which preferably is lenalidomide.

34. The method of claim 31, wherein the T lymphocytes are T lymphocytes in which the expression of the endogenous T cell receptor has been inhibited or eliminated.

35. The method of claim 1, wherein the ACTR construct is introduced into the T lymphocytes or the NK cells by a method selected from the group consisting of retroviral transduction, lentiviral transduction, DNA electroporation, and RNA electroporation.

36. The method of claim 1, wherein the T lymphocytes or NK cells expressing the ACTR construct are co-administered with an immunomodulatory agent, which preferably is lenalidomide.

37. The method of claim 1, wherein the anti-BCMA antibody is a human or humanized antibody.

38. The method of claim 1, wherein the anti-BCMA antibody contains an Fc variant fragment, which has an elevated binding affinity to a wild-type Fc receptor.

39. The method of claim 38, wherein the Fc variant fragment is afucosylated and/or contains one or more mutations in the hinge and/or CH2 domain of the Fc fragment.

40. The method of claim 1, wherein the anti-BCMA antibody binds to the same epitope in BCMA as antibody hSG16.17 or hSG16.45, or competes against antibody hSG16.17 or hSG16.45 from binding to BCMA.

41. The method of claim 40, wherein the anti-BCMA antibody comprises the same heavy chain complementary determining regions (CDRs) and light chain CDRs as antibody hSG16.17 or hSG16.45.

42. The method of claim 41, wherein the anti-BCMA antibody comprises the same heavy chain variable domain (V_{H}) and the same light chain variable domain (V_{L}) as antibody hSG16.17 or hSG16.45.

43. The method of claim 1, wherein the anti-BCMA antibody comprises an IgG1 heavy chain constant region.

44. A kit for immunotherapy, comprising:
(i) a pharmaceutical composition that comprises an anti-BCMA antibody, and a pharmaceutically acceptable carrier, wherein the anti-BCMA antibody is a humanized antibody; and
(ii) a population of T lymphocytes or NK cells that express an antibody-coupled T-cell receptor (ACTR) construct as defined in claim 1.

45. The kit of claim 44, further comprises an immunomodulatory agent, which preferably is lenalidomide.


SEQUENCE LISTING (118 sequences)

SEQ ID NO: 1 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIISFFLALTSTALLFLLFFLTLRFSVVKRGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLIALVTSGALLAVLGITGYFLMNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLAALLALLAALLALLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 428 aa, Artificial Sequence
  Synthetic Polypeptide
MALPVTALLLPLALLLHAARPQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 391 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYILDAILFLYGIVLTLLYCRLKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLLILLGVLAGVLATLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDITLGLLVAGVLVLLVSLGVAIHLCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVSFCLVMVLLFAVDTGLYFSVKTNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVAAILGLGLVLGLLGPLAILLALYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYLLDGILFIYGVILTALFLRVKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 441 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVMSVATIVIVDICITGGLLLLVYYWSKNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGFLFAEIVSIFVLAVGVYFIAGQDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGIIVTDVIATLLLALGVFCFAGHETKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVIGFRILLLKVAGFNLLMTLRLWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 438 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIVAVVIATAVAAIVAAVVALIYCRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVLFYLAVGIMFLVNTVLWVTIRKEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIILVGTAVIAMFFWLLLVIILRTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLGWLCLLLLPIPLIVWVKRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIAIYCIGVFLIACMVVTVILCRMKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 476 aa, Artificial Sequence
  Synthetic Polypeptide
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 623 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 403 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEAFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTSTEEGTSTEPSEGSAIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 433 aa, Artificial Sequence
  Synthetic Polypeptide
MLRLLLALNLFPSIQVTGGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, Artificial Sequence
  Synthetic Polypeptide
METDTLLLWVLLLWVPGSTGDGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 431 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 452 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQLGLHIWQLRSQCMWPRETQLLLEVPPSTEDARSCQFPEEERGERSAEEKGRLGDLWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 454 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCVKRRKPRGDVVKVIVSVQRKRQEAEGEATVIEALQAPPDVTTVAVEETIPSFTGRSPNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 453 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCYKVGFFKRNLKEKMEAGRGVPNGIPAEDSEQLASGQEAGDPGCLKPLHEKDSESGGGKDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 365 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDPQLCYILDAILFLYGIVLTLLYCRLKIQVRKAAITSYEKSDGVYTGLSTRNQETYETLKHEKPPQKRGRKKLLYIFKQPFMRPVQTTQEEDGCSCRFPEEEEGGCEL

SEQ ID NO: 56 - protein, 366 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 57 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 390 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 483 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 472 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 474 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 433 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 69 aa, Artificial Sequence
  Synthetic Polypeptide
TTTPAPRPPTPAPTIASQPLSLRPEACRPAAGGAVHTRGLDFACDIYIWAPLAGTCGVLLLSLVITLYC

SEQ ID NO: 65 - protein, 42 aa, Artificial Sequence
  Synthetic Polypeptide
KRGRKKLLYIFKQPFMRPVQTTQEEDGCSCRFPEEEEGGCEL

SEQ ID NO: 66 - protein, 112 aa, Artificial Sequence
  Synthetic Polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 21 aa, Artificial Sequence
  Synthetic Polypeptide
MALPVTALLLPLALLLHAARP

SEQ ID NO: 68 - protein, 121 aa, Artificial Sequence
  Synthetic Polypeptide
QVNLLQSRAALVKPGASVKLSCEASGYTFTDYYIHWVKQSHGKSLEWIGYINPNSGYTKYNENFKTKATMTADKSTNTAYVELSRLTSEDSATYFCTRYMWERVTGFFDFWGPGTKVTVSS

SEQ ID NO: 69 - protein, 121 aa, Artificial Sequence
  Synthetic Polypeptide
QVQLVQSRAEVKKPGASVKLSCKASGYTFTDYYIHWVRQAPGQGLEWIGYINPNSGYTKYNENFKTRATMTADKSINTAYVELSRLRSDDTAVYFCTRYMWERVTGFFDFWGQGTMVTVSS

SEQ ID NO: 70 - protein, 208 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 71 - protein, 184 aa, Homo sapiens [sequence omitted]

SEQ ID NO: 72 - protein, 254 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 73 - protein, 121 aa, Artificial Sequence
  Synthetic Polypeptide
QVQLVQSGAEVKKPGASVKLSCKASGYTFTDYYMHWVRQAPGQGLEWIGRINPNSGYTNYAQKFQGRVTMTADKSINTAYVELSRLRSDDTAVYFCTRYMWERVTGFFDFWGQGTMVTVSS

SEQ ID NO: 74 - protein, 121 aa, Artificial Sequence
  Synthetic Polypeptide
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 75 - protein, 121 aa, Artificial Sequence
  Synthetic Polypeptide
QVQLVQSGAEVKKPGASVKVSCKASGYTFTDYYMHWVRQAPGQGLEWIGRINPNSGYTNYAQKFQGRATMTADKSINTAYVELSRLRSDDTAVYFCTRYMWERVTGFFDFWGQGTMVTVSS

SEQ ID NO: 76 - protein, 300 aa, Artificial Sequence
  Synthetic Polypeptide
  misc_feature (16)..(300): may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 77 - protein, 15 aa, Artificial Sequence
  Synthetic Polypeptide
GGGGSGGGGSGGGGS

SEQ ID NO: 78 - protein, 30 aa, Artificial Sequence
  Synthetic Polypeptide
GGGGSGGGGSGGGGSGGGGSGGGGSGGGGS

SEQ ID NO: 79 - protein, 45 aa, Artificial Sequence
  Synthetic Polypeptide
GGGGSGGGGSGGGGSGGGGSGGGGSGGGGSGGGGSGGGGSGGGGS

SEQ ID NO: 80 - protein, 60 aa, Artificial Sequence
  Synthetic Polypeptide
GGGGSGGGGSGGGGSGGGGSGGGGSGGGGSGGGGSGGGGSGGGGSGGGGSGGGGSGGGGS

SEQ ID NO: 81 - protein, 75 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 82 - protein, 150 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 83 - protein, 225 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 84 - protein, 300 aa, Artificial Sequence; identical to SEQ ID NO: 76
  Synthetic Polypeptide

SEQ ID NO: 85 - protein, 20 aa, Artificial Sequence
  Synthetic Polypeptide
ETQRCTWHMGELVWCEREHN

SEQ ID NO: 86 - protein, 20 aa, Artificial Sequence
  Synthetic Polypeptide
KEASCSYWLGELVWCVAGVE

SEQ ID NO: 87 - protein, 13 aa, Artificial Sequence
  Synthetic Polypeptide
DCAWHLGELVWCT

SEQ ID NO: 88 - protein, 121 aa, Artificial Sequence
  Synthetic Polypeptide
QVQLVQSGAEVKKPGASVKVSCKASGYTFTDYYIHWVRQAPGQGLEWIGYINPNSGYTKYNENFKTRATMTADKSTNTAYVELSSLRSEDTAVYFCTRYMWERVTGFFDFWGQGTMVTVSS

SEQ ID NO: 89 - protein, 121 aa, Artificial Sequence
  Synthetic Polypeptide
QVQLVQSRAEVKKPGASVKLSCKASGYTFTDYYMHWVRQAPGQGLEWIGIINPNSGYTSYAQKFQGRVTMTADKSTNTAYVELSSLRSEDTAVYFCTRYMWERVTGFFDFWGQGTMVTVSS

SEQ ID NO: 90 - protein, 108 aa, Artificial Sequence
  Synthetic Polypeptide
DIQMTQSPASLSASLGETVSIECLASEDISDDLAWYQQKSGKSPQVLVYTTSRLQDGVPSRFSGSGSGTRFSLKIIVMQPEDEADYFCQQTYKFPPTFGAGTRLDLKR

SEQ ID NO: 91 - protein, 108 aa, Artificial Sequence
  Synthetic Polypeptide
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKR

SEQ ID NO: 92 - protein, 108 aa, Artificial Sequence
  Synthetic Polypeptide
DIQMTQSPSSVSASVGDRVTITCRASEDISDDLAWYQQKPGKAPKVLVYTTSSLQSGVPSRFSGSGSGTDFTLTISSLQPEDFATYFCQQTYKFPPTFGGGTKVEIKR

SEQ ID NO: 93 - protein, 108 aa, Artificial Sequence
  Synthetic Polypeptide
DIQMTQSPSSVSASVGDRVTITCLASEDISDDLAWYQQKPGKAPKVLVYTTSRLQSGVPSRFSGSGSGTDFTLTISSMQPEDFADYFCQQTYKFPPTFGGGTKVEIKR

SEQ ID NO: 94 - protein, 108 aa, Artificial Sequence
  Synthetic Polypeptide
DIQMTQSPSSVSASVGDRVTITCRASEDISDDLAWYQQKSGKAPKVLVYTTSSLQSGVPSRFSGSGSGTDFTLTISSLQPEDFATYFCQQTYKFPPTFGGGTKVEIKR

SEQ ID NO: 95 - protein, 116 aa, Artificial Sequence
  Synthetic Polypeptide
EVQLVESGGGLVQPGRSLKLSCVASGFTFNDHWMTWIRQAPGRGLEWISSITNTGGATYYLDSVKGRFTISRDNAKSTLYLQMNSLRSEDTATYYCTSPGLYFDYWGQGVMVTVSS

SEQ ID NO: 96 - protein, 116 aa, Artificial Sequence
  Synthetic Polypeptide
EVQLLESGGGLVQPGGSLRLSCAASGFTFNDHWMTWIRQAPGKGLEWISSITNTGGATYYLDSVKGRFTISRDNSKNTLYLQMNSLRAEDTAVYYCTSPGLYFDYWGQGVMVTVSS

SEQ ID NO: 97 - protein, 116 aa, Artificial Sequence
  Synthetic Polypeptide
EVQLLESGGGLVQPGGSLRLSCAASGFTFNDHWMTWIRQAPGKGLEWISAITNTGGATYYADSVKGRFTISRDNSKNTLYLQMNSLRAEDTAVYYCTSPGLYFDYWGQGVMVTVSS

SEQ ID NO: 98 - protein, 116 aa, Artificial Sequence
  Synthetic Polypeptide
EVQLLESGGGLVQPGGSLRLSCAASGFTFNDHWMTWIRQAPGKGLEWISAITNTGGATYYADSVKGRFTISRDNSKSTLYLQMNSLRAEDTAVYYCTSPGLYFDYWGQGVMVTVSS

SEQ ID NO: 99 - protein, 116 aa, Artificial Sequence
  Synthetic Polypeptide
EVQLLESGGGLVQPGGSLRLSCAASGFTFNDHWMTWVRQAPGKGLEWISSITNTGGATYYADSVKGRFTISRDNSKSTLYLQMNSLRAEDTAVYYCTSPGLYFDYWGQGTMVTVSS

SEQ ID NO: 100 - protein, 116 aa, Artificial Sequence
  Synthetic Polypeptide
EVQLVESGGGLVQPGGSLRLSCAASGFTFNDHWMTWVRQAPGKGLVWVSSITNTGGATYYADSVKGRFTISRDNAKNTLYLQMNSLRAEDTAVYYCTSPGLYFDYWGQGTMVTVSS

SEQ ID NO: 101 - protein, 116 aa, Artificial Sequence
  Synthetic Polypeptide
EVQLVESGGGLVQPGRSLRLSCAASGFTFNDHWMTWVRQAPGKGLEWVSGITNTGGATYYADSVKGRFTISRDNAKNSLYLQMNSLRAEDTALYYCTSPGLYFDYWGQGTMVTVSS

SEQ ID NO: 102 - protein, 107 aa, Artificial Sequence
  Synthetic Polypeptide
EIVLTQSPTTTAASPGEKVTITCLATSSVSVMYWYQHKSGASPKLLIYSTSSLASGVPDRFSGSGSGTSYSLTINTMEAEDAATYYCHQWSSDPPTFGSGTKLEIKR

SEQ ID NO: 103 - protein, 107 aa, Artificial Sequence
  Synthetic Polypeptide
EIVLTQSPGTLSASPGERVTISCRASSSVSVMYWYQHKPGQAPRLLIYSTSSLASGVPDRFSGSGSGTDYTLTISRMEPEDFAVYYCHQWSSDPPTFGQGTKLEIKR

SEQ ID NO: 104 - protein, 107 aa, Artificial Sequence
  Synthetic Polypeptide
EIVLTQSPGTLSLSPGERATLSCRASSSVSVMYWYQQKPGQAPRLLIYSTSSLASGIPDRFSGSGSGTDFTLTISRLEPEDFAVYYCHQWSSDPPTFGQGTKLEIKR

SEQ ID NO: 105 - protein, 107 aa, Artificial Sequence
  Synthetic Polypeptide
EIVLTQSPGTLSLSPGERATISCRASSSVSVMYWYQHKPGQAPRLLIYSTSSLASGVPDRFSGSGSGTDYTLTISRLEPEDFAVYYCHQWSSDPPTFGQGTKLEIKR

SEQ ID NO: 106 - protein, 107 aa, Artificial Sequence
  Synthetic Polypeptide
EIVLTQSPGTLSLSPGERATLSCLATSSVSVMYWYQHKPGQAPRLLIYSTSSLASGIPDRFSGSGSGTDYTLTISRLEPEDFAVYYCHQWSSDPPTFGQGTKLEIKR

SEQ ID NO: 107 - protein, 5 aa, Artificial Sequence
  Synthetic Polypeptide
  misc_feature (4)..(4): Xaa is Ile or Met
DYYXH

SEQ ID NO: 108 - protein, 5 aa, Artificial Sequence
  Synthetic Polypeptide
DHWMT

SEQ ID NO: 109 - protein, 17 aa, Artificial Sequence
  Synthetic Polypeptide
  misc_feature (1)..(1): Xaa is Tyr, Arg, or Ile
  misc_feature (10)..(10): Xaa is Lys, Asn, or Ser
  misc_feature (12)..(12): Xaa is Asn or Ala
  misc_feature (13)..(13): Xaa is Gln or Glu
  misc_feature (14)..(14): Xaa is Lys or Asn
  misc_feature (16)..(16): Xaa is Gln or Lys
  misc_feature (17)..(17): Xaa is Thr or Gly
XINPNSGYTXYXXXFXX

SEQ ID NO: 110 - protein, 17 aa, Artificial Sequence
  Synthetic Polypeptide
  misc_feature (1)..(1): Xaa is Ser, Ala, or Gly
  misc_feature (12)..(12): Xaa is Leu or Ala
XITNTGGATYYXDSVKG

SEQ ID NO: 111 - protein, 12 aa, Artificial Sequence
  Synthetic Polypeptide
YMWERVTGFFDF

SEQ ID NO: 112 - protein, 7 aa, Artificial Sequence
  Synthetic Polypeptide
PGLYFDY

SEQ ID NO: 113 - protein, 11 aa, Artificial Sequence
  Synthetic Polypeptide
  misc_feature (1)..(1): Xaa is Leu or Arg
XASEDISDDLA

SEQ ID NO: 114 - protein, 10 aa, Artificial Sequence
  Synthetic Polypeptide
  misc_feature (1)..(1): Xaa is Arg or Leu
  misc_feature (3)..(3): Xaa is Ser or Thr
XAXSSVSVMY

SEQ ID NO: 115 - protein, 7 aa, Artificial Sequence
  Synthetic Polypeptide
  misc_feature (4)..(4): Xaa is Ser or Arg
  misc_feature (7)..(7): Xaa is Asp or Ser
TTSXLQX

SEQ ID NO: 116 - protein, 7 aa, Artificial Sequence
  Synthetic Polypeptide
STSSLAS

SEQ ID NO: 117 - protein, 9 aa, Artificial Sequence
  Synthetic Polypeptide
QQTYKFPPT

SEQ ID NO: 118 - protein, 9 aa, Artificial Sequence
  Synthetic Polypeptide
HQWSSDPPT